(19) United States
(12) Patent Application Publication
(10) Pub. No.: US 2006/0217782 A1
(43) Pub. Date: Sep. 28, 2006

(54) Method and system for cortical stimulation to provide adjunct (ADD-ON) therapy for stroke, tinnitus and other medical disorders using implantable and external components

(76) Inventors: Birinder R. Boveja, Milwaukee, WI (US); Angely Widhany, Milwaukee, WI (US)

Correspondence Address: BIRINDER R. BOVEJA & ANGELY WIDHANY, P. O. BOX 210095, MILWAUKEE, WI, 53221, US

(21) Appl. No.: 11/445,692

(22) Filed: Jun. 2, 2006

Related U.S. Application Data
(63) Continuation-in-part of application No. 11/346,684, filed on Feb. 3, 2006.
Application No. 11/346,684 is a continuation of application No. 10/195,961, filed on Jul. 16, 2002, now Pat. No. 7,062,330.
Application No. 10/195,961 is a continuation of application No. 09/752,083, filed on Dec. 29, 2000, now Pat. No. 6,505,074.
Application No. 09/752,083 is a continuation-in-part of application No. 09/178,060, filed on Oct. 26, 1998, now Pat. No. 6,205,359.
Application No. 11/346,684 is a continuation-in-part of application No. 10/841,995, filed on May 8, 2004, now Pat. No. 7,076,307.
Application No. 10/841,995 is a continuation-in-part of application No. 10/196,533, filed on Jul. 16, 2002.
Application No. 10/841,995 is a continuation-in-part of application No. 10/142,298, filed on May 9, 2002.

(51) Int. Cl. A61N 1/18

(52) U.S. Cl. 607/45

(57) ABSTRACT

A method and system for providing rectangular and/or complex electrical pulses to cortical tissues of a patient for providing therapy or alleviating symptoms for at least one of tinnitus, essential tremor (ET) including Parkinson's disease, depression, or for providing improvement of functional recovery following stroke. The intracranial electrodes may be implanted on the dura, or subdurally on the pia mater of the cortical surface. The placement of the electrodes may utilize cortical sensing and/or digital imaging techniques, such as fMRI, MRI, or CT. The pulse generator system comprises implantable and external components. The pulse generator may be an implanted pulse generator (IPG) or an external stimulator coupled with an implanted stimulus-receiver. The IPG may also comprise a rechargeable battery. In one embodiment the pulse generator may also comprise a selected number of predetermined/pre-packaged programs. In one embodiment, the pulse generation may also comprise telemetry means, for remote interrogation and/or programming of said pulse generator utilizing a wide area network, such as the internet.

This application is a continuation-in-part of application Ser. No. 11/346,684 entitled “Method and system for cortical stimulation with rectangular and/or complex electrical pulses to provide therapy for stroke and other neurological disorders”, filed on Feb. 3, 2006, which is a continuation of application Ser. No. 10/195,961 which is a continuation of Ser. No. 09/752,083 which is a Continuation-in Part of application Ser. No. 09/178,060 now U.S. Pat. No. 6,205,359 filed Oct. 26, 1998. application Ser. No. 11/346,684 is also a CIP of application Ser. No. 10/841,995, which is CIP of application Ser. No. 10/196,533 which is a CIP of application Ser. No. 10/142,298. Priority is claimed from these applications, and the prior Applications being incorporated herein by reference.

### FIELD OF INVENTION

The present invention relates to brain stimulation, more specifically to cortical stimulation for providing improvement of functional recovery following stroke including stroke related aphasia, and to provide adjunct (add-on) therapy for other neurological diseases such as tinnitus, Parkinson's disease, and depression using rectangular and/or complex electrical pulses.

### BACKGROUND

This patent disclosure is directed to providing rectangular and/or complex electrical pulses to the cortical areas in the brain. One objective of supplying electrical pulses to the cortical areas is for inducing or enhancing neuroplasticity, where other areas of the brain take over the function of stroke-damaged areas. By providing subthreshold cortical stimulation during the rehabilitation process, the improvement of functional recovery following stroke is significantly improved, including for stroke related aphasia (impaired ability to speak). Subthreshold cortical stimulation is defined as that stimulation level which does not evoke movement and cannot be felt by the patient. Cortical stimulation as disclosed in this patent application may also be used to provide therapy or alleviate symptoms of other neurological disorders such as tinnitus, essential tremors (ET) including Parkinson's disease, and depression.

One or more leads are implanted with the electrodes in proximity to the cortical surface of the brain, with the electrodes being either subdural or epidural. fMRI or other imaging tools may also be used to aid in the proper location placement of the cortical electrodes. The terminal portion of the lead is tunneled subcutaneously to a convenient location, such as behind the ear or the pectoral or axillary region. The terminal end of the lead is connected to a pulse generator means, which is then implanted subcutaneously or submuscularly. The pulse generator may also be external.

The pulse generator system may be one from a group comprising:

a) an implanted stimulus-receiver used with an external stimulator;

b) an implanted stimulus-receiver comprising a high value capacitor for storing charge, used in conjunction with an external stimulator;

c) a programmer-less implantable pulse generator (IPG) which is operable with a magnet;

d) a microstimulator;

e) a programmable implantable pulse generator;

f) a combination implantable device comprising both a stimulus-receiver and a programmable IPG; and

g) an IPG comprising a rechargeable battery.

Use of any of these systems for providing cortical pulses is considered with the scope of this disclosure.

### Background of Stroke

Stroke is a cardiovascular disease that affects the blood vessels supplying blood to the brain. Stroke occurs when a blood vessel that carries oxygen and nutrients to the brain is clogged by a blood clot or bursts. Because of this blockage or rupture, part of the brain does not get the blood flow it needs. Deprived of oxygen, nerve cells in the affected area of the brain cannot function and die within minutes. When nerve cells cannot function, the part of the body controlled by these cells cannot function either. The devastating effects of stroke are often permanent because dead brain cells cannot be replaced.

Stroke is the third leading cause of death among Americans and probably first as a cause of chronic functional incapacity. Approximately two million people in the United States today are impaired by the neurological consequences of cerebrovascular disease. Many of them are between 25 and 64 years of age. Every year there are in this country approximately 700,000 cases of stroke—roughly 600,000 ischemic lesions and 100,000 hemorrhages, intracerebral or subarachnoid—with 175,000 fatalities from these causes. Since 1950, coincident with the introduction of effective treatment for hypertension, there has been a substantial reduction in the frequency of stroke.

Stroke is an acute focal neurologic deficit from a vascular disorder that injures brain tissue. There are two main types of strokes: ischemic stroke and hemorrhagic stroke. Ischemic strokes are caused by cerebrovascular obstruction by thrombosis or emboli, and are the most common type of stroke, accounting for 70% to 80% of all strokes. Focal cerebral ischemia follows reduction or cessation of blood flow to a localized area of the brain due to large-vessel disease (such as embolic or thrombotic arterial occlusion, often in a setting of atherosclerosis). Although most occlusive strokes are due to atherosclerosis and thrombosis and most hemorrhagic strokes are associated with hypertension or aneurysms, strokes of either type may occur at any age from many causes, including cardiac disease, trauma, infection, neoplasm, blood dyscrasia, vascular malformation, immunological disorder, and exogenous toxins.

The less common hemorrhagic strokes are caused by bleeding into brain tissue. Hemorrhagic stroke occurs with rupture of a blood vessel, hemorrhage into the brain tissue occurs, resulting in edema, compression of the brain contents. This type of stroke usually is from a blood vessel rupture caused by hypertension, aneurysms, arteriovenous malformations, head injury, or blood dyscrasias and has much higher fatality rate than ischemic strokes.

More than any other organ, the brain depends from moment to moment on an adequate supply of oxygenated blood. Constancy of the cerebral circulation is assured by a series of baroreceptors and vasomotor reflexes under the control of centers in the lower brainstem. In humans, the complete stoppage of blood flow for longer than 5 minutes produces irreversible damage. Brain tissue deprived of blood undergoes ischemic necrosis or infarction.

Neuronal function is affected in two stages during ischemia. Neuronal electrical function is lost when the blood flow falls below a critical threshold of approximately 20 mL of blood per 100 g of brain tissue per minute. At this level, brain tissue is thought to be revivable, with the potential to reverse ischemic damage. However, irreversible damage occurs when blood flow falls below 10 mL of blood per 100 g of brain tissue per minute. Inefficient anaerobic metabolism of glucose occurs which rapidly leads to lactic acidosis and failure of the normal energy-dependent cellular ion homeostasis. Potassium leaves the cell, and sodium and water enter the cell and lead to cytotoxic edema. Calcium also enters the cell and sets a cascade of molecular events into motion that eventually leads to neuronal death.

Pharmaceutical treatment for stroke utilizes, drugs that may enhance activity-dependent gains include, amphetamine, piracetam, and cholinergic and dopaminergic agents have suggested efficacy of these agents for particular aphasic syndromes and language impairments.

### Neural Plasticity

Neural plasticity is the capacity of the nervous system to change. Neural plasticity is obvious during the development of neural circuits, however, the adult brain also posses substantial plasticity in order to learn new skills, establish new memories, and respond to injury throughout life. In adult brains the altered neural function in maturity appears to rely primarily on carefully regulated changes in the strength of existing synapses. Extensive changes can occur when the adult nervous system is damaged by trauma or disease. It is known that new neurons can be generated throughout life in a limited number of brain regions, whereby new cells can be integrated into existing circuits.

Biomedical research with primates has also supported this. The four cortical areas that define the primate somatic sensory cortex (Broadmann's areas 3a, 3b, 1, and 2) each contain a complete topographic representation of the body surface. J. Kaas and M. Merzenich took advantage of this arrangement by carefully defining the normal spatial organization of topographic maps in these regions. They then amputated a digit (or cut one of the nerves that innervated the hand) and reexamined topographical maps in the same animals several weeks later. Surprisingly, the somatic sensory cortex had changed: The cortical neurons that had been deprived of their normal peripheral input now responded to stimulation of other parts of the animal's hand. For example, if the third digit was amputated, cortical neurons that formerly responded to stimulation of digit 3 responded to stimulation of digits 2 or 4. Thus, the central representation of the remaining digits had expanded to take over the cortical territory that had lost its main input. Such “functional re-mapping” also occurs in the somatic sensory nuclei in the thalamus and brainstem; indeed, some of the reorganization of cortical circuits may depend on this concurrent subcortical plasticity. This sort of adjustment in the somatic sensory system may contribute to the altered sensation of phantom limbs after amputation. Similar plastic changes now have been demonstrated in the visual, auditory, and motor cortices, suggesting that some ability to reorganize after peripheral deprivation or injury is a general property of the mature neocortex

### Tinnitus

In some applications the cortical stimulation methods and systems of this current disclosure may also be used to provide therapy or alleviate symptoms of tinnitus. Tinnitus is defined as any abnormal sound in the head, which may be chronic and often intense. By some estimates, as much as 32% of the adult population has tinnitus, with 20% rating their condition as severe. Tinnitus may be considered a significant symptom when its intensity so overrides normal environmental sounds that it invades consciousness. The patient experiencing tinnitus may describe the sound as ringing, roaring, hissing, whistling, chirping, rustling, clicking, or buzzing. Although most patients report the presence of tinnitus as constant, others report it as intermittent, fluctuating, or pulsating. Tinnitus may be perceived as a high- or a low-pitched tone, a band of noise, or some combination of such sounds.

The perceived loudness of tinnitus in any patient may be sufficiently intense to be disquieting. Tinnitus is a symptom of an underlying disease or specific lesion when it is perceived above the intensity levels of environmental sounds. Severe tinnitus may disable individuals to the extent that they cannot concentrate on anything other than the tinnitus itself.

In the auditory system, sound impinging on the ear is transmitted through a mechanical system including the tympanic membrane and ossicular chain, ending at the stapes. Sound energy is then converted into changes in neural firing, which is passed more centrally through a complex cross-connected network of neurons. This neural network can be considered as a series of four order of neurons, as is shown in conjunction with FIG. 1A. First order neurons are derived from the auditory (spiral) ganglion; second order neurons are those in the neighborhood of the cochlear nuclei in the brain stem. Third order neurons are found at the level of the inferior colliculus, and fourth order neurons travel from the medial geniculate body in the thalamus to the auditory cortex 31.

Medical research has shown efficacy of cortical stimulation to provide therapy for tinnitus, which may be used in conjunction with other therapies such as pharmacological therapy including medications such as carbamazepine, intravenous lidocaine or barbiturates, antianxiety drugs such as diazepam and alprazolam, or antidepressants such as amitriptyline.

### Parkinson's Disease and Movement Disorders

In some applications the cortical stimulation methods and systems of this current disclosure may be used to provide therapy or alleviate symptoms of Essential Tremer (ET) or other movement disorders such as Parkinson's disease.

Parkinson's disease (PD) belongs to a group of conditions called motor system disorders, which are the result of the loss of dopamine producing brain cells. Parkinsonism is the most common movement disorder in adults affecting 1% to 2% of patients>60 years old. Parkinson's disease (PD) is a progressive neurodegenerative disorder whose pathologic hallmark is loss of dopaminergic neurons in the substantia nigra pars compacta. The cardinal motor signs of PD are tremor, rigidity, bradykinesia, akinesia, and a gait disorder characterized by a flexed posture and short, shuffling steps. Patients may also develop postural instability and freezing, a phenomenon characterized by a sudden inability to continue or initiate movement. Decreased associated movements (masked facies, decreased eye blink, and reduced arm swing) are common early signs of PD. Hyprophonia, micrographia, and difficulty with fine motor control (buttoning buttons, handling utensils, shaving, or applying makeup), and getting out of a chair or rolling over in bed at night are common early complaints of PD patients.

The symptoms of PD are tremor, or trembling in hand, arms, legs, jaw, and face; rigidity, or stiffness of the limbs and trunk; bradykinesia, or slowness of movement; and postural instability, or impaired balance and coordination. As these symptoms become more pronounced, patients may have difficulty walking, talking, or completing other simple tasks. PD usually affects people over the age of 50. Early symptoms of PD are subtle and occur gradually. In some people the disease progresses more quickly than in others. As the disease progresses, the shaking, or tremor, which affects the majority of PD patients may begin to interfere with daily activities. Other symptoms may include depression and other emotional changes; difficulty in swallowing, chewing, and speaking; urinary problems or constipation; skin problems; and sleep disruptions.

There is no known treatment that will halt or reverse the neuronal degeneration that presumably underlies Pakinson's disease.

### Other Concomitant Disorders

Sleep disorders are common in Parkinsonian patients and may exacerbate Parkinsonian motor signs as a result of the excessive fatigue and daytime sleepiness.

Depression is a common occurrence in patients with PD, but it is often overlooked. It has been clinically observed that successful treatment of their depression is almost always associated with a concurrent improvement in parkinsonian motor signs.

### Other Movement Disorders

Essential tremor (ET), which is the most common movement disorder is an insidiously progressive and often inheritable disorder usually beginning before the age of 50. The genetic basis is uncertain. It is characterized by involuntary rhythmic oscillations of a body part resulting from either alternating or synchronous contractions of opposing muscles. Tremor is essentially the only symptom present, although subtle gait abnormalities may be noted when the legs are affected. Weakness is not a primary symptom although tremor can produce weakness by reducing the strength of contraction.

Huntington's Disease (HD) is characterized as a triad of symptoms and signs: a movement disorder, a cognitive disorder, and a psychiatric disorder. Each of these domains may be problematic for the individual at various states of the illness, which on average spans 15 to 20 years.

Progressive supranuclear palsy (PSP) is the most common Parkinsonian disorder after Parkinson's disease (PD). Typically, PSP patients present with early postural instability, supranuclear vertical gaze palsy, and levodopa-nonresponsive parkinsonism (bradykinesia and axial more than limb rigidity).

### Existing Medical and Surgical Therapy

General Approach to Therapy: Initial medical treatment typically involves the use of drugs to replace striatal dopamine or drugs that have dopaminergic properties, e.g., dopamine agonists.

Disease Progression and Development of Motor Complication Wearing Off (End-of-Dose Phenomenon): Over time, patients' symptoms typically become more severe, and they begin to develop wearing-off phenomenon (i.e., symptoms return before the next dose of medication). When this occurs, one can increase the dose of medication, decrease the time interval between doses, add an agonist or begin a COMT inhibitor to minimize the amount of “off” time. There should be a small amount of time before the next dose when the patient notes some loss of effect because this indicates that the patient is not receiving more medication than necessary.

Patients with PD whose motor symptoms can no longer be controlled adequately be medical therapy are candidates for surgical therapy. Surgical procedures of PD consist of ablative procedures (thalamotomy, pallidotomy) and stimulation procedures (thalamic, pallidal, subthalamic).

Thalamotomy: Thalamotomy is effective for the treatment of parkinsonian tremor. Lesions are generally placed in the cerebellar receiving area, ventralis intermedius (VIM). If the lesion is extended more anteriorly into the basal ganglia receiving area, ventralis oralis posterior and ventralis oralis anterior. Thalamotomy may also improve rigidity and drug-induced dyskinesias.

Pallidotomy: Pallidotomy is effective for all the cardinal motor signs of PD, including tremor, rigidity, and bradykinesia, as well as motor fluctiations and drug-induced dyskinesias and dystomia. It may also improve axial symptoms, including gait, balance, and freezing. The improvement in axial symptoms after unilateral pallidotomy, however, is less consistent than that for appenducular symptoms, with many patients losing their benefit anywhere form 6 months to 2 years postpallidotomy.

Deep Brain Stimulation (DBS): DBS in the GPi or the STN for PD can be performed either as a staged procedure or simultaneously. Simultaneous procedures may be associated with a higher incidence of postoperative confusion. Based on the patient's symptoms, unilateral implantaion may benefit the patient enough to preclude or at least delay the necessity for a second implantation on the other side. Most patients, however, will require bilateral implantation to gain optimal control over axial symptoms or to gain bilateral control over appendicular symptoms. Both targets, the GPi and the STN, are effective in treating the cardinal motor signs of PD, including gait, balance, and freezing symptoms.

### Depression

In some applications the cortical stimulation methods and systems of this disclosure may be used to provide therapy or alleviate the symptoms of depression. Depression is a very common disorder that is often chronic or recurrent in nature. It is associated with significant adverse consequences for the patient, patient's family, and society. Among the consequences of depression are functional impairment, impaired family and social relationships, increased mortality from suicide and comorbid medical disorders, and patient and societal financial burdens. Depression is the fourth leading cause of worldwide disability and is expected to become the second leading cause by 2020.

One of the most important distinctions between mood disorders is the distinction between unipolar and bipolar categories. Unipolar mood disorders are characterized by depressive symptoms in the absence of a history of pathologically elevated mood. In bipolar mood disorders, depression alternates or is mixed with mania or hypomania.

With respect to cortical function, depression involves multiple distrubances of information processing. The neurocognitive changes of depression point to dysfunction involving the hippocampus, prefrontal cortex, and other limbic structures.

Major depressive disorder is associated with a myriad of neurobiological disturbances. The changes in brain function assosciated with severe depression include increased phasic REM sleep, poor sleep maintenance, hypercortisolism, impaired cellular immunity, reductions of anterior cerebral blood flow and glucose metabolism, and increased glucose metabolism in the amygdala.

Among the currently available treatment modalities include, pharmacotherapy with antidepressant drugs (ADDs), specific forms of psychotherapy, and electroconvulsive therapy (ECT). ADDs are the usual first line treatment for depression. Commonly the initial drug selected is a selective serotonin reuptake inhibitor (SSRI) such as fluoxetine (Prozac), or another of the newer ADDs such as venlafaxine (Effexor).

Several forms of psychotherapy are used to treat depression. Among these, there is good evidence for the efficacy of cognitive behavior therapy and interpersonal therapy, but these treatments are used less often than are ADDs. Phototherapy is an additional treatment option that may be appropriate monotherapy for mild cases of depression that exhibit a marked seasonal pattern

Many patients do not respond to initial antidepressant treatment. Furthermore, many treatments used for patients who do not respond at all, or only respond partially to the first or second attempt at antidepressant therapy are poorly tolerated and/or are associated with significant toxicity. For example, tricyclic antidepressant drugs often cause anticholinergic effects and weight gain leading to premature discontinuation of therapy, and they can by lethal in overdose (a significant problem in depressed patients). Lithium is the augmentation strategy with the best published evidence of efficacy (although there are few published studies documenting long-term effectiveness), but lithium has a narrow therapeutic index that makes it difficult to administer; among the risks associated with lithium are renal and thyroid toxicity. Monoamine oxidase inhibitors are prone to produce an interaction with certain common foods that results in hypertensive crises. Even selective serotonin reuptake inhibitors can rarely produce fatal reaction in the form of a serotonin syndrome.

Physicians usually reserve electroconvulsive therapy (ECT) for treatment-resistant cases or when they determine a rapid response to treatment is desirable. ECT is also associated with significant risks: long-lasting cognitive impairment following ECT significantly limits the acceptability of ECT as a long-term treatment for depression. Therefore, there is a strong need for non-pharmacological well-tolerated and effective long-term or maintenance treatments for patients who do not respond fully, or for patients who do not sustain a response to first-line pharmacological therapies.

### Background for Cortical Stimulation

A simplified general anatomy of the human brain is shown in conjunction with FIGS. 1B, 1C, 1D and will be referred to throughout the disclosure. FIG. 1B depicts the brain from a top view, highlighting the frontal lobe, parietal lobe, occipital lobe, as well as, highlighting the precentral gyrus 28 and postcentral gyrus 30 separated by the central sulcus 29. FIG. 1A depicts the brain from a lateral view highlighting the anatomical regions of interest in this patent disclosure, such as primary somatosensory cortex 37, primary motor cortex 36, and premotor cortex 35. Other cortical areas such as primary visual cortex, primary auditory cortex 31, and limbic and anterior association areas are also highlighted. FIG. 1D shows a perspective of the brain via a midsection view, again showing the region around the central sulcus 29, and some of deeper aspects of the brain tissues.

In the method and system of this invention, it will be appreciated that even though the electrodes are placed only on the cortical surface, the electrical field will penetrate deeper layers of the cortical brain tissue, depending upon the electrode configuration and placement of the electrodes as described later. The deeper layers of the cortex are depicted in an overview fashion in FIG. 2A, and a detail of the layers of cells is shown in conjunction with FIG. 2B, where the layer of pyramidal cells is shown as layer III.

The human brain has been mapped to a significant extent. In the early part of the twentieth century, K. Brodmann divided the human cerebral cortex into 52 discrete areas on the basis of distinctive nerve cell structures and characteristic arrangements of cell layers, as is shown in conjunction with FIG. 3. Brodmann's scheme of the cortex is still widely used today and is continually updated. In this drawing (FIG. 3) each area is represented by its own symbol and is assigned a unique number. Several areas defined by Brodmann have been found to control specific brain functions. For instance, area 4, the motor cortex, is responsible for voluntary movement. Areas 1, 2, and 3 comprise the primary somatosensory cortex, which receives information on bodily sensation. Area 17 is the primary visual cortex, which receives signals from the eyes and relays them to other areas for further deciphering. Areas 41 and 42 comprise the primary auditory cortex. Areas not visible from the outer surface of the cortex are not shown in this drawing.

Electrical stimulation has been used to identify the specific motor effects of discrete sites in the frontal lobe in humans, and the resulting motor maps have been correlated with anatomical and clinical observations on the effects of local lesions. The contralateral precentral gyrus (Brodmann's area 4), the region now called the primary motor cortex, proved to be the area in which the lowest intensity stimulation elicited movements.

By stimulating motor cortical areas in alert humans by inducing electrical fields in the brain using rapidly alternating magnetic fields produced by wire coils applied to the scalp. The responses in muscles (e.g. of the hand) are recorded with surface electrodes. The motor action potentials are large and have a short latency, consistent with the fact that they are conducted by corticospinal fibers, shown in FIG. 4. Magnetic stimulation has also been used to map the body representation in the primary motor cortex or to perturb processing in local cortical areas.

Initially, a simplistic idea was believed that the primary motor cortex acts as a massive switchboard with individual switches controlling individual muscles or small groups of adjacent muscles. More detailed studies, however, using microelectrodes inserted into the depths of the cortex (intracortical microstimulation or ICMS) to stimulate small groups of output neurons indicate that this simple view is incorrect. Whereas the weakest stimuli may evoke the concentration of individual muscles, the same muscles are invariably activated from several separate sites as well, indicating that neurons in several cortical sites project axons to the same target. This provides the basis for cortical stimulation to enhance neuroplasticity in post-stroke patients, as disclosed in this patent application.

In addition, most stimuli activate several muscles, with muscles rarely being activated individually. This is corroborated by recent anatomical and physiological experiments showing that the terminal distributions of individual corticospinal axons diverge to motor neurons innervating more than one muscle. Instead of a simple switchboard of muscle representation, detailed maps of monkey motor cortex suggest a concentric organization: sites influencing distal muscles are contained at the center of a wider area containing sites that also influence more proximal muscles, while sites in the peripheral ring around this central area influence proximal muscles alone. An implication of the redundancy in muscle representation is that inputs to motor cortex from other cortical can combine proximal and distal muscles in different ways in different tasks.

FIG. 5A depicts the cerebral cortex with simplified map of functional areas labeled on the cerebral cortex. FIG. 5B depicts further details of motor cortical areas which are of interest in this patent disclosure particularly as pertaining to stroke and Parkinson's disease. FIG. 5B highlights primary somatic sensory cortex, primary motor cortex, supplementary motor area, and premotor cortex. These functional areas are superimposed with Broadman's area shown in FIG. 3.

The motor maps show an orderly arrangement along the gyrus of control areas for the face, digits, hand, arm, trunk, leg, and foot. However, the fingers, hands, and face—which are used in tasks requiring the greatest precision and finest control—have disproportionately large representations in the motor areas of cortex (FIG. 5B), much as the inputs from regions of the body that have important roles in perception predominate in the sensory areas of the cortex. Consistent with the overall somatotopic organization, lesions in the arm representation lead to degeneration of myelinated fibers in the cervical cord, while lesions in the leg representation produce degeneration extending all the way to the lumbar spinal cord. These are Betz cells whose axons arise from specialized large pyramidal neurons in lamina V.

It is known that different areas of the cortex are activated during simple, complex, and imagined sequences of finger movements. Local increases in cerebral blood flow during a behavior indicate which areas of motor cortex are involved in the behavior, since local tissue perfusion varies with neural activity. For example, as shown in conjunction with FIG. 6A, when a finger is pressed repeatedly against a spring, increased blood flow is detected in the hand-control areas of the primary motor and sensory cortices. The increase in the motor area is related to the execution of the response, whereas the increase in the sensory area reflects the activation of peripheral receptors. Shown in conjunction with FIG. 6B, during a complex sequence of finger movements the increase in blood flow extends to the medial premotor area, which includes the supplementary motor area and presupplementary motor area. Shown in conjunction with FIG. 6C, during mental rehearsal of the same sequence illustrated in FIG. 6B, blood flow increases only in the medial motor area.

An overall map of the convoluted outer layer of gray matter that forms the cerebral cortex is shown in conjunction with FIG. 7A. The larger the area, the more nerve cells and fibers it contains. The diagram separates the breakdown of the motor cortex (on the right side of the figure's brain, controlling the left side of the body) from that of the sensory cortex (left brain, right-hand side of the body). The two “stripes” are located on the whole brain, at the bottom portion of FIG. 7A. For clarity, a somatotopic map of the body surface onto primary somatosensory cortex is shown in conjunction with FIG. 7B. This map is a cross section through the postcentral gyrus, shown at the top of figure. Neurons in each area are most responsive to the parts of the body illustrated above them. FIG. 7C shows a somatotopic map of the human precentral gyrus (primary motor cortex). Systematic probing of this region has shown that there is a somatotopic organization in the human precentral gyrus much like that seen in the somatosensory areas of the postcentral gyrus.

### Background Cellular Neurophysiology

At the cellular level, nerve cells have membranes that are composed of lipids and proteins (shown schematically in FIGS. 8A and 8B), and have unique properties of excitability such that an adequate disturbance of the cell's resting potential can trigger a sudden change in the membrane conductance. Under resting conditions, the inside of the nerve cell is approximately −90 mV relative to the outside. The electrical signaling capabilities of neurons are based on ionic concentration gradients between the intracellular and extracellular compartments. The cell membrane is a complex of a bilayer of lipid molecules with an assortment of protein molecules embedded in it (shown in FIG. 8A), separating these two compartments. Electrical balance is provided by concentration gradients which are maintained by a combination of selective permeability characteristics and active pumping mechanism.

The lipid component of the membrane is a double sheet of phospholipids, elongated molecules with polar groups at one end and the fatty acid chains at the other. The ions that carry the currents used for neuronal signaling are among these water-soluble substances, so the lipid bilayer is also an insulator, across which membrane potentials develop. In biophysical terms, the lipid bilayer is not permeable to ions. In electrical terms, it functions as a capacitor, able to store charges of opposite sign that are attracted to each other but unable to cross the membrane. Embedded in the lipid bilayer is a large assortment of proteins. These are proteins that regulate the passage of ions into or out of the cell. Certain membrane-spanning proteins allow selected ions to flow down electrical or concentration gradients or by pumping them across.

These membrane-spanning proteins consist of several subunits surrounding a central aqueous pore (shown in FIG. 8B). Ions whose size and charge “fit” the pore can diffuse through it, allowing these proteins to serve as ion channels. Hence, unlike the lipid bilayer, ion channels have an appreciable permeability (or conductance) to at least some ions. In electrical terms, they function as resistors, allowing a predicable amount of current flow in response to a voltage across them.

FIG. 9A illustrates a segment of the surface of the membrane of an excitable cell. Metabolic activity maintains ionic gradients across the membrane, resulting in a high concentration of potassium (K⁺) ions inside the cell and a high concentration of sodium (Na⁺) ions in the extracellular environment. The net result of the ionic gradient is a transmembrane potential that is largely dependent on the K⁺ gradient. Typically in nerve cells, the resting membrane potential (RMP) is slightly less than 90 mV, with the outside being positive with respect to inside.

To stimulate an excitable cell, it is only necessary to reduce the transmembrane potential by a critical amount. When the membrane potential is reduced by an amount ΔV, reaching the critical or threshold potential (TP); Which is shown in FIG. 9B. When the threshold potential (TP) is reached, a regenerative process takes place: sodium ions enter the cell, potassium ions exit the cell, and the transmembrane potential falls to zero (depolarizes), reverses slightly, and then recovers or repolarizes to the resting membrane potential (RMP).

For a stimulus to be effective in producing an excitation, it must have an abrupt onset, be intense enough, and last long enough. These facts can be drawn together by considering the delivery of a suddenly rising cathodal constant-current stimulus of duration d to the cell membrane as shown in FIG. 9B.

Cell membranes can be reasonably well represented by a capacitance C, shunted by a resistance R as shown by a simplified electrical model in the diagram in FIG. 9C, and shown in a more realistic electrical model in FIG. 10, where neuronal process is divided into unit lengths, which is represented in an electrical equivalent circuit. Each unit length of the process is a circuit with its own membrane resistance (rₘ), membrane capacitance (cₘ), and axonal resistance (rₐ).

A nerve cell can be excited by increasing the electrical charge within the neuron, thus increasing the membrane potential inside the nerve with respect to the surrounding extracellular fluid. As shown in FIG. 11A, stimuli 43 and 44 are subthreshold, and do not induce a response. Stimulus 45 exceeds a threshold value and induces an action potential (AP) 27 which will be propagated. The threshold stimulus intensity is defined as that value at which the net inward current (which is largely determined by Sodium ions) is just greater than the net outward current (which is largely carried by Potassium ions), and is typically around −55 mV inside the nerve cell relative to the outside (critical firing threshold). If however, the threshold is not reached, the graded depolarization will not generate an action potential and the signal will not be propagated along the axon. This fundamental feature of the nervous system i.e., its ability to generate and conduct electrical impulses, can take the form of action potentials 27, which are defined as a single electrical impulse passing down an axon. This action potential 27 (also called nerve impulse or spike) is an “all or nothing” phenomenon, that is to say once the threshold stimulus intensity is reached, an action potential will be generated. Depicted in conjunction with FIG. 11B, the information in the nervous system is coded by frequency and pattern of firing rather than the size of the action potential.

As is well known in the art, the operation of the nervous system depends on the flow of information through chains of neurons functionally connected by synapses. Most synapses occur between the axon terminal of one neuron and the dendrite or cell body of a second neuron. Sometimes, however, synapses occur between two dendrites or between a dendrite and a cell body or between an axon terminal and a second axon terminal to modulate its output. A neuron that conducts a signal toward a synapse is a presynaptic neuron, whereas a neuron conducting signals away from a synapse is a postsynaptic neuron. FIG. 12A shows how, in a multineuronal pathway, a single neuron can be postsynaptic to one cell and presynaptic to another. A post synaptic neuron may have thousands of synaptic junctions on the surface of its dendrites and cell body, so that signals from many presynaptic neurons can affect it.

There are variety of synapse contacts. Although most synapses in the nervous system occur between axons and dendrites, synaptic contact can occur at any region of the neuron. For example, the somas of nearly all cells in the central nervous system (CNS) receive synapses from axons. Shown in conjunction with FIG. 12B, synapses can be formed between almost any neuronal structures. The most common sites for synaptic contact are between axon boutons and neuron somas (A), between two axons (B), and between axon boutons and dendrites (C). Much less common are synapses between dendrites and somas (D) and between two dendrites (E).

Most CNS neurons receive thousands of synaptic inputs. The transformation of many synaptic inputs to a single neuronal output constitutes neural computation. The brain performs billions of neural computations every second.

The simplest form of synaptic integration in the CNS is excitatory postsynaptic potential (EPSP) summation. Excitatory postsynaptic potentials (EPSPs) are local graded depolarization events which occur at excitatory post synaptic membranes. The function of EPSPs is to help trigger an action potential distally at the axon hillock of the postsynaptic neuron. Shown in conjunction with FIG. 13A, there are two types of summation: Spatial and Temporal, and typically they occur together. Spatial summation is the adding together of EPSPs generated simultaneously at many different synapses on a dendrite. Temporal summation is the adding together of EPSPs generated at the same synapse if they occur in rapid succession, within 1-15 msec of one another.

When summation results from buildup of neurotransmitter released simultaneously by several presynaptic end bulbs, it is spatial summation, shown in the left part of FIG. 13A. When summation results from buildup of neurotransmitter released by a single presynaptic end bulb two or more times in rapid succession, it is temporal summation, shown in the right part of FIG. 13A. As a typical EPSP lasts about 15 msec, the second (and subsequent) release of neurotransmitter must occur soon after the first one if temporal summation is to occur.

A single postsynaptic neuron receives input from many presynaptic neurons, some of which release excitatory neurotransmitters and some of which release inhibitory neurotransmitters. The sum of all the excitatory and inhibitory effects at any given time determines the effect on the postsynaptic neuron, which may respond in the following ways:
1. EPSP. If the total excitatory effects are greater than the total inhibitory effects but less than the threshold level of stimulation, the result is a subthreshold EPSP. Subsequent stimuli can more easily generate a nerve impulse through summation because the neuron is partially depolarized.
2. Nerve impule(s). If the total excitatory effects are greater than the total inhibitory effects and the threshold level of stimulation is reached or surpassed, the EPSP spreads to the initial segment of the axon and triggers one or more nerve impulses. Impulses continue to be generated as long as the EPSP stays above the threshold level.
3. IPSP. If the total inhibitory effects are greater than the excitatory effects, the membrane hyperpolarizes (IPSP). The result is inhibition of the postsynaptic neuron and an inability to generate a nerve impulse

Similarly, inhibitory postsynaptic potentials (IPSPs) also summate, both temporally and spatially. In the case of IPSPs, the postsynaptic neuron is inhibited to a greater degree. Most neurons receive both stimulatory and inhibitory inputs from thousands of other neurons.

A typical neuron in the CNS receives input from 1000-10,000 synapses. Integration of these inputs occurs at the trigger zone. The greater the summation of EPSPs, the greater the chance that threshold will be reached and a nerve impulse will be initiated.

Shown in conjunction with FIG. 13B, synapses that produce IPSPs tend to be concentrated at the base of large dendrites and on the soma of neurons. Generally speaking, the excitatory synapses are more distal. Consequently, proximal inhibitory synapses have more influence on the membrane potential at the initial segment than the distal excitatory synapses. By providing a low-resistance pathway, or sink, for electrical current to leave the cell, the IPSP can short-circuit the positive currents generated by EPSPs. The smooth integration of excitatory and inhibitory events over time and space ultimately determines whether or not the postsynaptic cell initiates an action potential. This integration brings together information from diverse sources and at a single moment transforms this collective set of information into a single postsynaptic event, the action potential. The summation of synaptic currents, both excitatory and inhibitory, at the initial segment of the axons is the fundamental decision-making process of the nervous system.

The physical relation between synaptic boutons that produce EPSPs and IPSPs affects the amplitude and time course of the postsynaptic potential as recorded at the soma of the neuron. As shown in FIG. 13B, the graph portion at the bottom of the figure illustrates a hypothetical computer-generated EPSPs at the base of a dendrite which are calculated for three physical arrangements;
A. an EPSP generated at the indicated site produces a response at the initial segment as shown by the broken black line in the graph. A simultaneous IPSP applied at a site distal to the EPSP hardly affects the amplitude or shape of the EPSP (solid red line).
B. If the EPSP and the IPSP are simultaneously generated at the same site along the dendrite, the amplitude of the EPSP is diminished by about half and its time course is slowed.
C. If the IPSP-generating synaptic bouton is between the recording site and the EPSP-generating synaptic bouton, the inhibitory effect is even more profound.

FIG. 14 depicts a more realistic model of neuron as an integrative structure approaching the complexity of the neurons pictured in FIGS. 2A and 2B, replete with an enormous and complex dendritic tree and a nonuniform distribution of ion channels. The responses of a neuron like this reflect the principles of spatiotemporal summation. Such a neuron receives thousands or tens of thousands of synaptic contacts distributed over its entire dendritic tree, the cell body, and on the axon near terminal boutons, as shown in FIG. 14. The voltage-sensitive sodium channels critical to the initiation of an action potential are preferentially distributed in a region at the base of the axon known as the initial segment, which is immediately adjacent to the axon hillock. The transmission of electrical impulses along the axon is “all or none” because it takes advantage of the self-perpetuating dynamics of the action potential: once an action potential is generated at the axon hillock, it regenerates itself all the way down the axon (3E in FIG. 14). However, transmission of impulses along dendritic branches is passive and therefore subject to the attenuating effects of fiber resistance and capacitance. Therefore, synaptic contacts that are on large dendritic branches (low resistance) are going to have more influence on the neuron than synaptic contacts on small branches (high resistance), and synaptic contacts near the cell body (or even better, the initial segment near the axon hillock) (input A, FIG. 14) are going to have more influence than synaptic contacts distant in the dendritic tree (input B, FIG. 14). Further, synaptic contacts may be excitatory (e.g., glutamate, generating EPSPs) or inhibitory (e.g., GABA, generating IPSPs) (as in input C, FIG. 14). Synaptic contacts on the axon terminal are capable of modifying the amount of neurotransmitter emitted by an action potential, something referred to as presynaptic excitation or inhibition (input D, FIG. 14).

Any given neuron produces action potentials at a rate that reflects a spatially weighted integral of all its inputs over time—that is, spatiotemporal summation. This arrangement seems to provide neurons with the potential for enormous information processing sophistication.

FIG. 15 summarizes a graphical representation of spatial and temporal summation at synaptic junctions. (a) When presynaptic neurons A and A separately cause EPSPs (arrows) in postsynaptic neuron C, the threshold level is not reached in neuron C. Spatial summation occurs only when neurons A and B act simultaneously on neuron C; Their EPSPs sum to reach the threshold level and trigger a nerve impulse. (b) Temporal summation occurs when stimuli applied to the same axon in rapid succession (arrows at the bottom of the graph) cause overlapping EPSPs that sum. When depolarization reaches the threshold level, a nerve impulse is triggered.

Shown in conjunction with FIG. 16A, at the cellular level by passing continuous depolarizing current into a neuron through a microelectrode 493A, 493B, many action potentials are generated in succession. Further, as shown in conjunction with FIG. 16B the rate of action potential generation in a single cell depends on the magnitude of the continuous depolarizing current. As shown in FIG. 16B, in the left part 492 the injected current does not depolarize the membrane to threshold, and no action potentials are generated. In the middle portion 494 of FIG. 16B, the injected current depolarizes the membrane beyond threshold, and some action potentials are generated. In the right portion 496 of FIG. 16B, as the injected current increases, the firing rate of action potentials also increases.

### Tissue Stimulation

In the method and system of this invention, this concept is applied at the tissue level, where as shown in conjunction with FIG. 17, instead of injecting current into a cell, the stimulating current (electrical pulses) is/are applied to the cortical tissues at the relevant portions of the brain. In certain situations it would be desirable to apply suprathreshold electrical stimulation, and in certain other situations it would be desirable to apply subthreshold (low level) electrical stimulation. Subthreshold cortical stimulation is defined as that stimulation level which does not evoke movement and cannot be felt by the patient.

In some applications, an objective of this invention is to provide subthreshold stimulation to target tissues using cortical electrodes, to either enhance or induce neuroplasticity, for providing improvement of functional recovery following stroke. The subthreshold stimulation leads to partially depolarized neurons, which are more easily prone to action potentials, because they are already nearer to threshold. It is expected that cortical stimulation of the healthy brain tissue adjacent to the “stroke,” in combination with rehabilitation, enhances motor recovery and that cortical stimulation for stroke patients will facilitate neuroplasticity. This approach will lead to synaptic and morphologic changes associated with activity-dependent plasticity at the levels of the cerebral hemispheres.

Recovery of function after stroke is associated with a series of changes in the motor cortex that allow uninjured areas of the cortex to compensate for functions lost due to the stroke. When the brain is stimulated with a small amount of electric current during rehabilitation therapy, it is believed that it makes it easier for the brain to form new connections and relearn lost motor skills.

Surgery is done to open the skull. A small grid is implanted on the covering of the brain (the dura), which in turn covers the area of motor cortex. The motor cortex is the part of the brain that controls movement. A wire connected to the grid sticks out of the patient's head. In one embodiment, during therapy sessions the wire is connected to a battery pack in a vest, as described later. The electrical stimulator stimulates the grid and, therefore, the motor cortex. While the nerves that were killed in the stroke will not regain function, new connections in the brain can be made and that's what is expected to happen. Also, research shows that the stimulation increases blood flow to the part of the brain that is being stimulated. Patients have therapy for a period of time determined by the physician.

Another objective of this invention is to provide cortical stimulation, to provide therapy or alleviate symptoms for other neurological disorders/diseases such as tinnitus, Parkinson's disease, depression, and other neurological disorders that are amenable to brain stimulation utilizing one or more implanted cortical electrodes and a pulse generator means.

### Relevant Art

U.S. Pat. No. 6,959,215 B2 (Gliner et al.) is generally directed to methods for treating essential tremor.

U.S. Patent Applications 0097161 A1 (Firlik et al.), 0091419 (Firlik et al.), 0130706 A1 (Sheffield et al.), 0021105 μl (Firlik et al.), and 0087201 A1 (Firlik et al.) are all generally directed to methods and apparatus for effectuating a lasting change in a neural-function of a patient.

### SUMMARY OF THE INVENTION

The method and system of current invention provides pulsed electrical stimulation to the cortical portion of a patient's brain to provide therapy or alleviate symptoms of neurological disorders such as tinnitus, essential tremor (ET) including Parkinson's disease, depression, or for providing improvement of functional recovery following stroke. The method and system comprises both implantable and external components. The power source may also be external or implanted in the body. The system to provide selective stimulation to the cortex may be selected from a group consisting of:

a) an implantable stimulus-receiver used in conjunction with an external stimulator;

b) an implantable stimulus-receiver comprising a high value capacitor for storing charge, used in conjunction with an external stimulator;

c) a programmer-less implantable pulse generator (IPG) which is operable with a magnet;

d) a microstimulator;

e) a programmable implantable pulse generator (IPG);

f) a combination implantable device comprising both a stimulus-receiver and a programmable. IPG; and

g) an IPG comprising a rechargeable battery.

In one aspect, rectangular and/or complex electrical pulses are provided to the cortical tissues of a patient, wherein complex electrical pulses comprise at least one of multi-level pulses, biphasic pulses, non-rectangular pulses, or pulses with varying amplitude during the pulse.

In another aspect, the electrical pulses are provided according to predetermined/pre-packaged programs

In another aspect, the electrode configuration for providing said electrical pulses is at least partly based on sensed electrical activity from the patient's cortical tissues.

In another aspect, the electrode placement on the patient's cortex is based at least in part to digital imaging techniques, such as fMRI or CT scans.

In another aspect, the electrode placement on the patient's cortex is based both on digital imaging techniques and on sensing from the cortical tissues of the patient.

In another aspect, the configuration of electrodes for providing electrical pulses is alternated between at least two configurations.

In another aspect, the predetermined/pre-packaged programs can be modified.

In another aspect, the range of electrical pulses comprises, pulse amplitude between 0.1 volt-25 volts; pulse width between 20 micro-seconds-5 milli-seconds; stimulation frequency between 5 Hz and 150 Hz, and blocking frequency between 100 and 1,000 Hz.

In yet another aspect, the pulse generation system comprises telemetry means and can be remotely interrogated and/or programmed over a wide area network.

Various other features, objects and advantages will be made apparent from the following description taken together with the drawings.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1A depicts four order of neurons of the human auditory system.

FIG. 1B depicts a top view of a human brain.

FIG. 1C depicts a lateral view of a human brain.

FIG. 1D depicts a midsection of a human brain.

FIG. 2A depict deeper layers of a section of the human cortex.

FIG. 2B is a close-up of a cross section of human cortex, showing six layers of cells.

FIG. 3 depicts a lateral view of a human brain, showing various Brodmann's area.

FIG. 4 depicts recordings from hand resulting from electrical stimulation of the human motor cortex.

FIG. 5A is a figure of a human brain depicting various broad functional areas correlating to different areas in the brain.

FIG. 5B depicts functional areas in the brain relating to voluntary movement, particularly motor cortex and sensory cortex.

FIGS. 6A, 6B, and 6C depicts different areas of human cortex which are activated during simple, complex, and imagined sequences of finger movements.

FIG. 7A depicts parts of the body that are correlated with sensory and motor areas of the human brain.

FIG. 7B depicts a map which is a cross section through the postcentral gyrus showing neurons in each area that are most responsive to the parts of the body illustrated above them.

FIG. 7C depicts a somatotopic map of the human precentral gyrus which corresponds to the motor cortex.

FIGS. 8A and 8B are schematic illustrations of the biochemical makeup of nerve cell membrane.

FIGS. 9A, 9B, 9C are schematic illustrations of the electrical properties of nerve cell membrane.

FIG. 10 is a schematic illustration of the electrical circuit model of nerve cell membrane.

FIG. 11A is a figure demonstrating subthreshold and suprathreshold stimuli.

FIG. 11B depicts subthreshold potentials and a train of action potentials.

FIG. 12A depicts an arrangement of nerve cells showing both presynaptic and postsynaptic junctions.

FIG. 12B depicts another form of synaptic arrangement between nerve cells.

FIG. 13A depicts in graphical form the spatial and temporal summation of excitatory postsynaptic potentials (EPSPs).

FIG. 13B depicts graphically summation of EPSPs and IPSPs.

FIG. 14 depicts another arrangement of nerve cells connecting and communicating to process information.

FIG. 15 depicts excitatory and inhibitory synapses, as well as, temporal and spatial summation for reaching threshold.

FIG. 16A depicts an injected current leading to action potentials.

FIG. 16B depicts the relationship between injected current and nerve cell firings of action potentials.

FIG. 17 is a figure that depicts an electrical field penetrating through the cells of the cortical layers.

FIG. 18 is a figure that depicts sensing from ischemic and “healthy” cortical tissues via multiple electrode pairs.

FIGS. 19A and 19B depict electrode configurations for stimulation of cortical tissues.

FIG. 20 depicts placement of stimulation electrodes relative to an ischemic region, based on imaging techniques.

FIG. 21 depicts placement of paddle stimulation electrodes which are implanted approximately perpendicular to each other

FIGS. 22A and 22B depict electrode configurations for stimulating cortical tissues.

FIG. 23A depicts placement of paddle electrodes on the motor cortex of the brain.

FIG. 23B depicts placement of electrodes on the motor cortex of the brain comprising two layers of electrodes.

FIG. 23C depicts placement of electrodes on the motor cortex of the brain comprising three layers of electrodes.

FIG. 24 is a simplified block diagram depicting supplying amplitude and pulse width modulated electromagnetic pulses to an implanted coil.

FIG. 25 depicts a customized garment for placing an external coil to be in close proximity to an implanted coil.

FIG. 26 is a diagram showing an implanted stimulus-receiver with the distal electrodes in proximity with the cortical tissues.

FIG. 27A depicts the placement of a primary coil of an external stimulator using a head band.

FIG. 27B depicts using eye glasses for placement of a primary coil in close proximity to a secondary coil which is implanted behind the ear.

FIG. 27C depicts using an ear piece for properly placing a primary coil of an external stimulator.

FIG. 28 is a schematic of the passive circuitry in the implanted stimulus-receiver.

FIG. 29A is a schematic of an alternative embodiment of the implanted stimulus-receiver.

FIG. 29B is another alternative embodiment of the implanted stimulus-receiver.

FIG. 30 shows coupling of the external stimulator and the implanted stimulus-receiver.

FIG. 31 is a top-level block diagram of the external stimulator and proximity sensing mechanism.

FIG. 32 is a diagram showing the proximity sensor circuitry.

FIG. 33 shows a pulse train that may be transmitted to the cortical tissues.

FIG. 34 is a schematic diagram of one embodiment of an implantable lead for supplying electrical pulses to the cortical tissues.

FIG. 35A is a schematic diagram showing the implantable lead and one form of stimulus-receiver.

FIG. 35B is a block diagram showing schematically the functioning of the external transmitter and the implanted stimulus-receiver of one embodiment.

FIG. 35C is a schematic diagram showing a workable Class-D driver.

FIGS. 35D and 35E are electrical diagrams showing the concept of Class-E amplifier.

FIG. 35F is a schematic diagram showing a workable Class-E driver.

FIG. 36 is a schematic block diagram showing a system for neuromodulation of the cortical tissues, with an implanted component which is both RF coupled and contains a capacitor power source.

FIG. 37 is a simplified block diagram showing control of the implantable neurostimulator with a magnet.

FIG. 38 is a schematic diagram showing implementation of a multi-state converter.

FIG. 39 is a schematic diagram depicting digital circuitry for state machine.

FIGS. 40A-C depicts various forms of implantable microstimulators.

FIG. 41 is a figure depicting an implanted microstimulator for providing pulses to cortical tissues.

FIG. 42 is a diagram depicting the components and assembly of a microstimulator.

FIG. 43 shows functional block diagram of the circuitry for a microstimulator.

FIGS. 44A, 44B, and 44C are simplified block diagrams of the implantable pulse generator.

FIG. 45 is a functional block diagram of a microprocessor-based implantable pulse generator.

FIG. 46 shows details of implantable pulse generator.

FIGS. 47A and 47B show details of digital components of the implantable circuitry.

FIG. 48A shows a schematic diagram of the register file, timers and ROM/RAM.

FIG. 48B shows datapath and control of custom-designed microprocessor based pulse generator.

FIG. 49 is a block diagram for generation of a pre-determined stimulation pulse.

FIG. 50 is a simplified schematic for delivering stimulation pulses.

FIG. 51 is a circuit diagram of a voltage doubler.

FIG. 52A is a diagram depicting ramping-up of a pulse train.

FIG. 52B depicts rectangular pulses.

FIGS. 52C, 52D, and 52E depict multi-step pulses.

FIGS. 52F, 52G, and 52H depict complex pulse trains.

FIG. 52-I depicts the use of tripolar electrodes.

FIGS. 52J and 52K depict step pulses used in conjunction with tripolar electrodes.

FIGS. 52L and 52M depict biphasic pulses used in conjunction with tripolar pulses.

FIGS. 52N and 52-O depict modified square pulses to be used in conjunction with tripolar electrodes.

FIGS. 53A and 53B are diagrams showing communication of programmer with the implanted stimulator.

FIGS. 54A and 54B show diagrammatically encoding and decoding of programming pulses.

FIG. 55 is a simplified overall block diagram of implanted pulse generator (IPG) programmer.

FIG. 56 shows a programmer head positioning circuit.

FIG. 57 depicts typical encoding and modulation of programming messages.

FIG. 58 shows decoding one bit of the signal from FIG. 57.

FIG. 59 shows a diagram of receiving and decoding circuitry for programming data.

FIG. 60 shows a diagram of receiving and decoding circuitry for telemetry data.

FIG. 61 is a block diagram of a battery status test circuit.

FIG. 62 is a diagram showing the two modules of the implanted pulse generator (IPG).

FIG. 63A depicts coil around the titanium case with two feedthroughs for a bipolar configuration.

FIG. 63B depicts coil around the titanium case with one feedthrough for a unipolar configuration.

FIG. 63C depicts two feedthroughs for the external coil which are common with the feedthroughs for the lead terminal.

FIG. 63D depicts one feedthrough for the external coil which is common to the feedthrough for the lead terminal.

FIG. 64 shows a block diagram of an implantable stimulator which can be used as a stimulus-receiver or an implanted pulse generator with rechargeable battery.

FIG. 65 is a block diagram highlighting battery charging circuit of the implantable stimulator of FIG. 64.

FIG. 66 is a schematic diagram highlighting stimulus-receiver portion of implanted stimulator of one embodiment.

FIG. 67A depicts bipolar version of stimulus-receiver module.

FIG. 67B depicts unipolar version of stimulus-receiver module.

FIG. 68 depicts power source select circuit.

FIG. 69A shows energy density of different types of batteries.

FIG. 69B shows discharge curves for different types of batteries.

FIG. 70 depicts externalizing recharge and telemetry coil from the titanium case.

FIGS. 71A and 71B depict recharge coil on the titanium case with a magnetic shield in-between.

FIG. 72 shows in block diagram form, an implantable rechargeable pulse generator.

FIG. 73 depicts in block diagram form, the implanted and external components of an implanted rechargeable system.

FIG. 74 depicts the alignment function of rechargeable implantable pulse generator.

FIG. 75 is a block diagram of the external re-charger.

FIG. 76 depicts remote monitoring of stimulation devices.

FIG. 77 is an overall schematic diagram of the external stimulator, showing wireless communication.

FIG. 78 is a schematic diagram showing application of Wireless Application Protocol (WAP).

FIG. 79 is a simplified block diagram of the networking interface board.

FIGS. 80A and 80B is a simplified diagram showing communication of modified PDA/phone with an external stimulator via a cellular tower/base station.

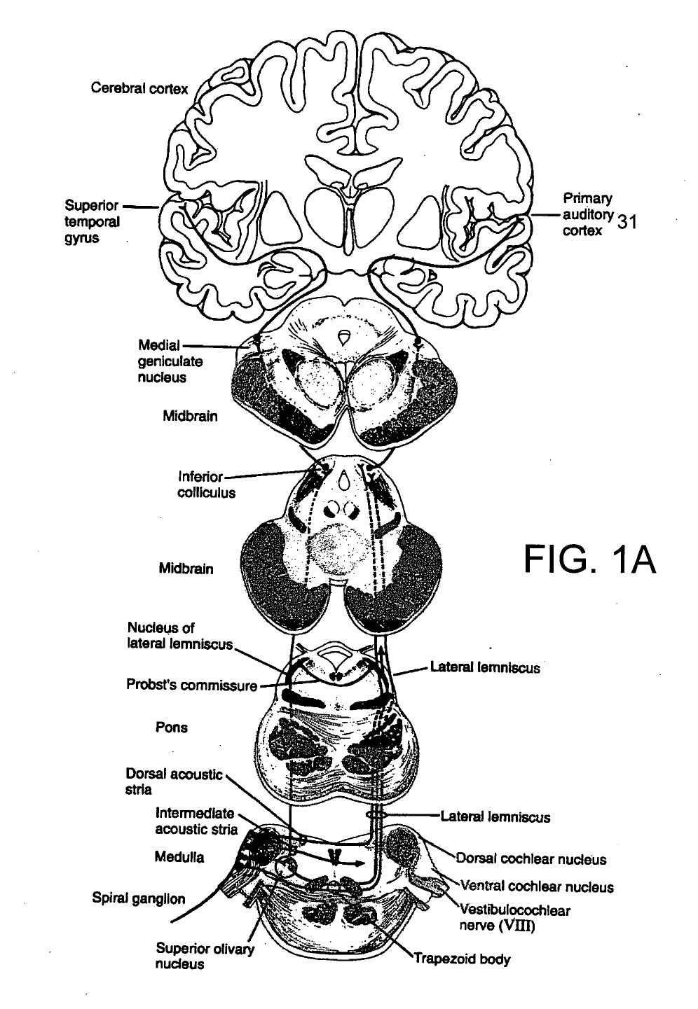

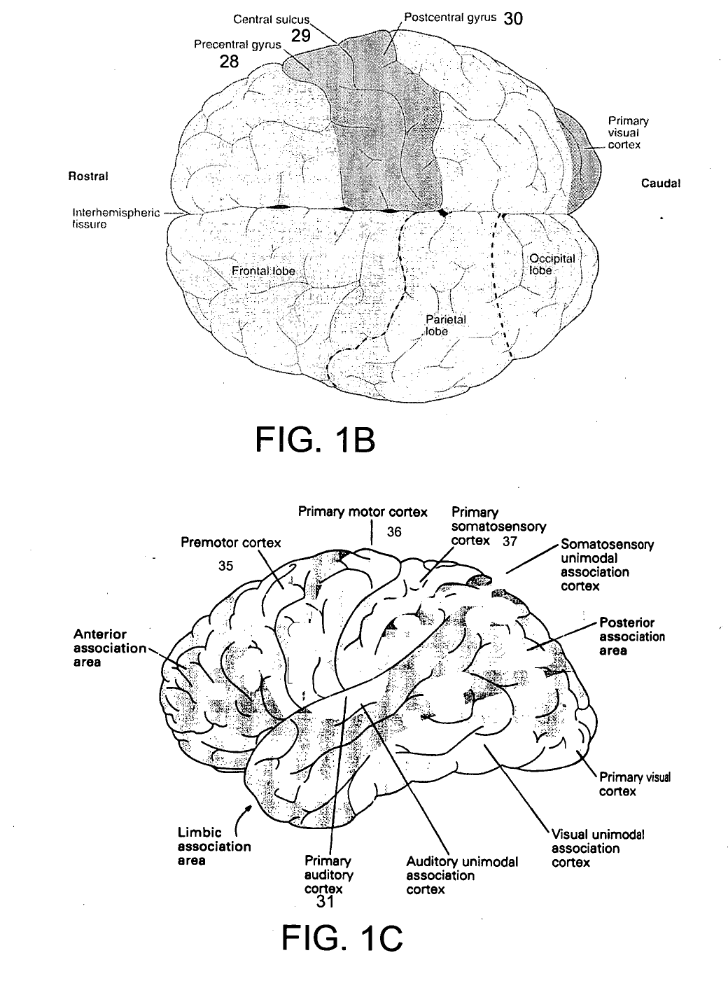

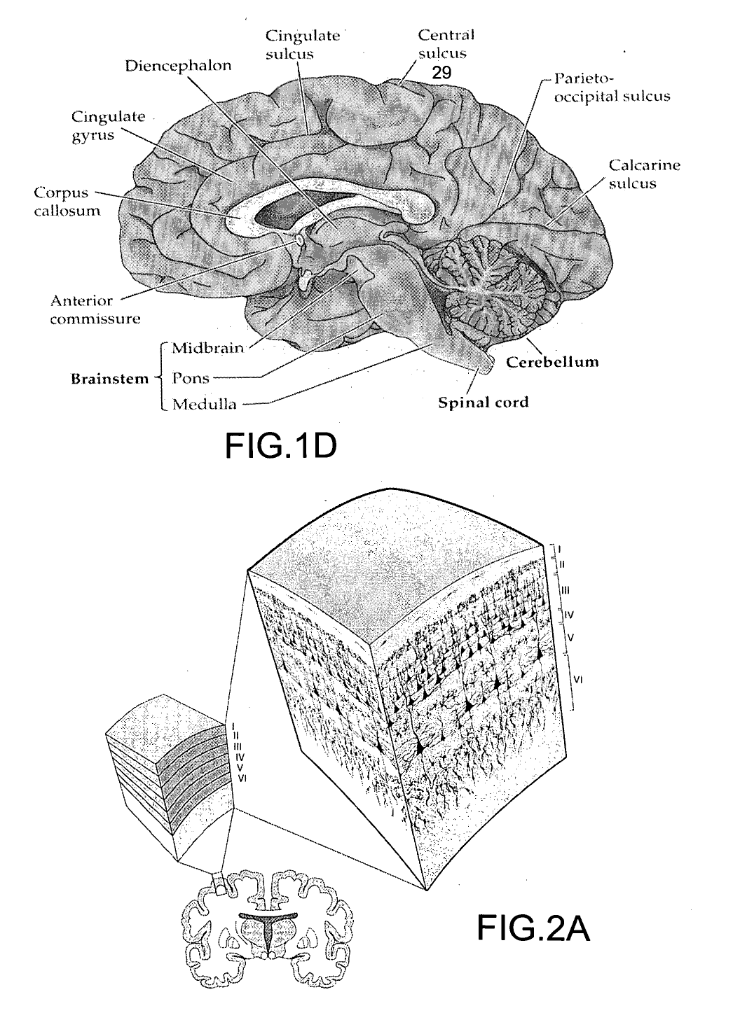

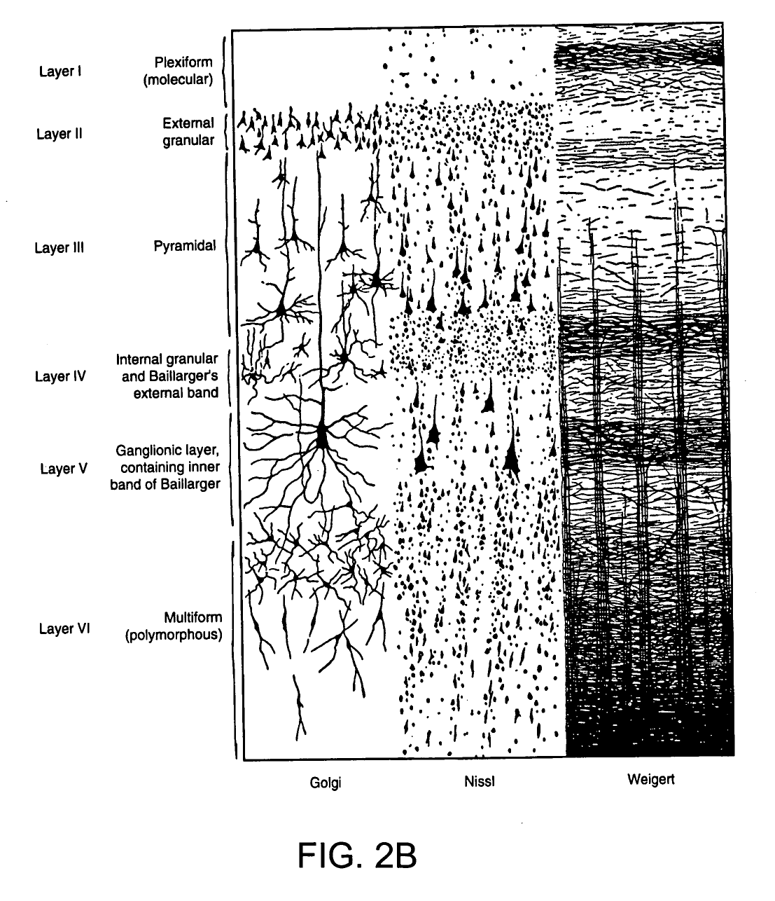

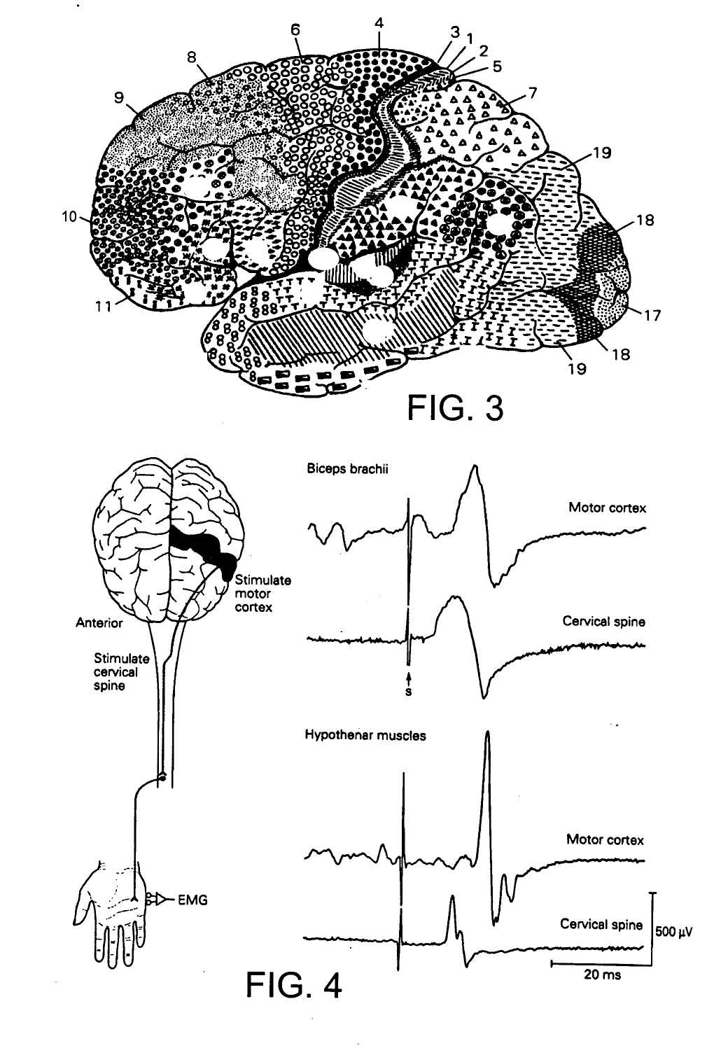

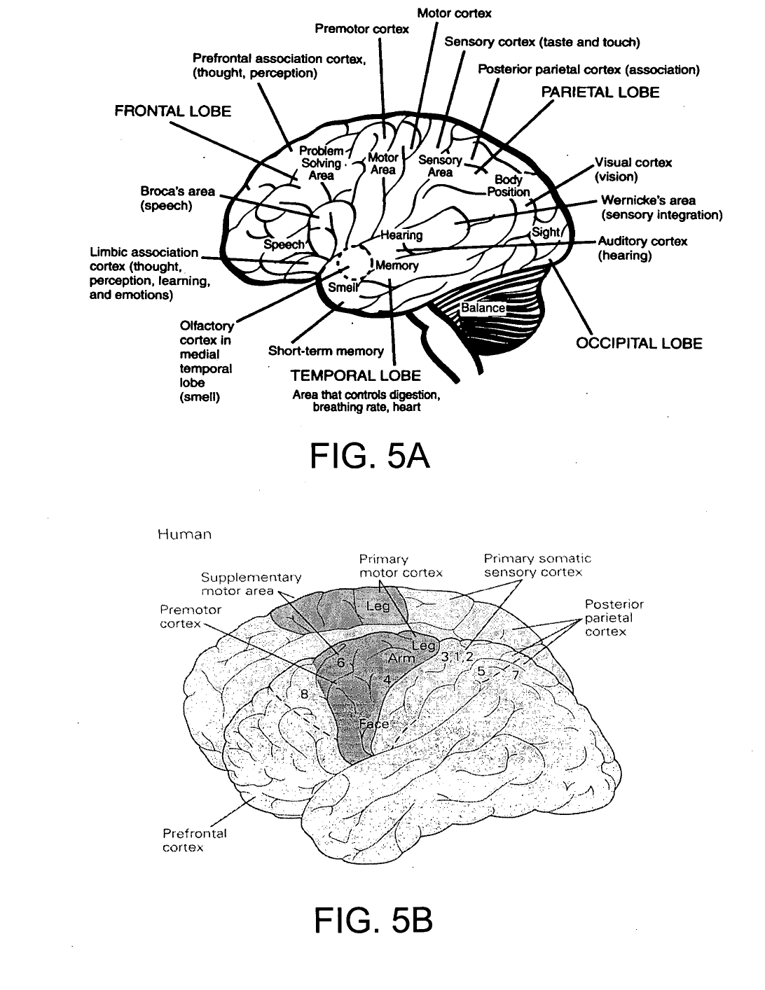

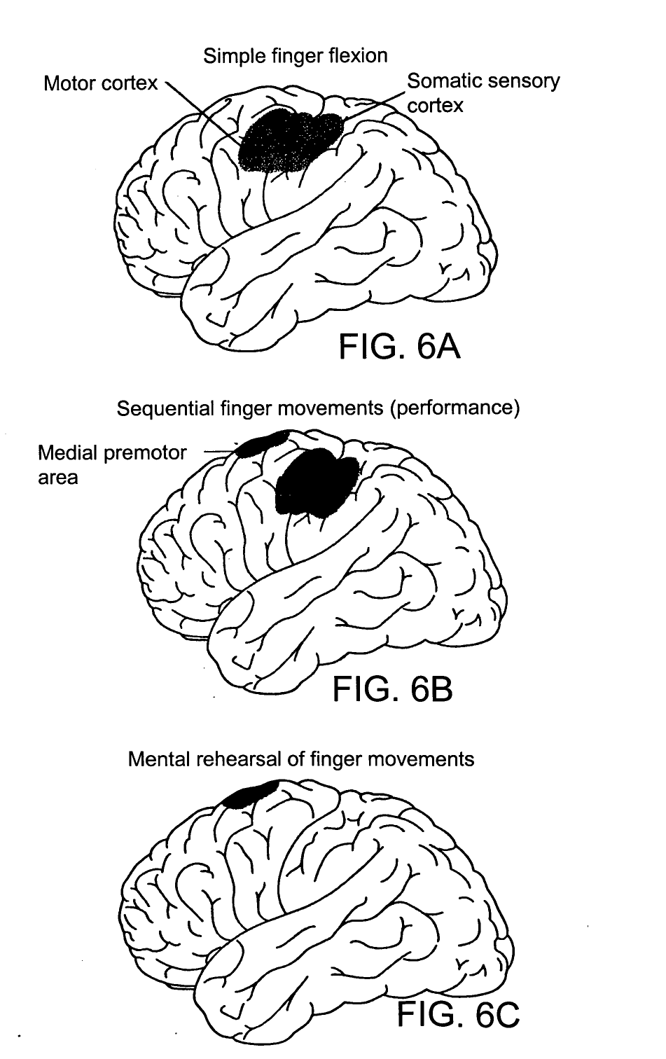

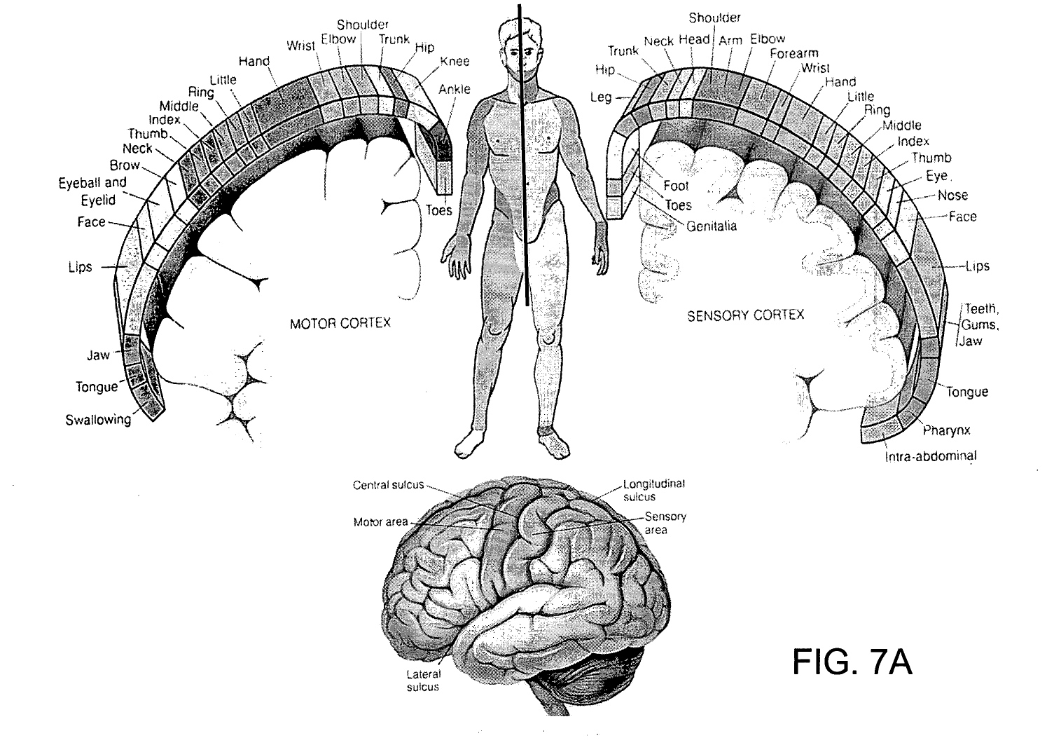

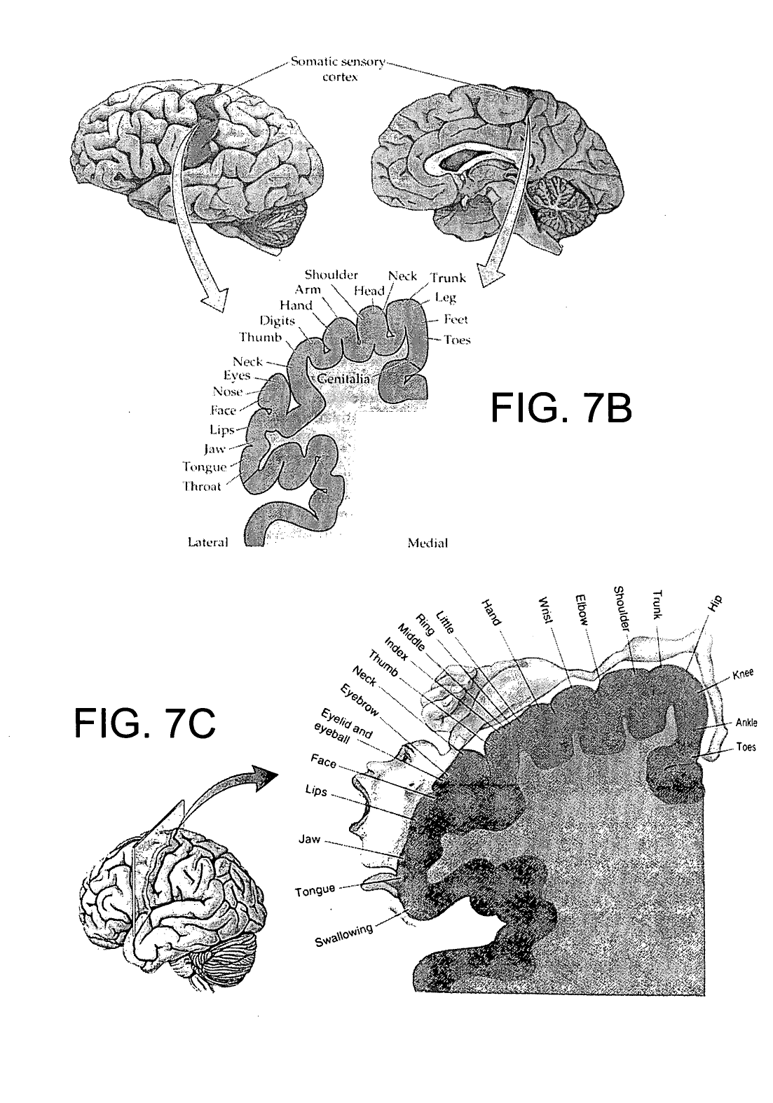

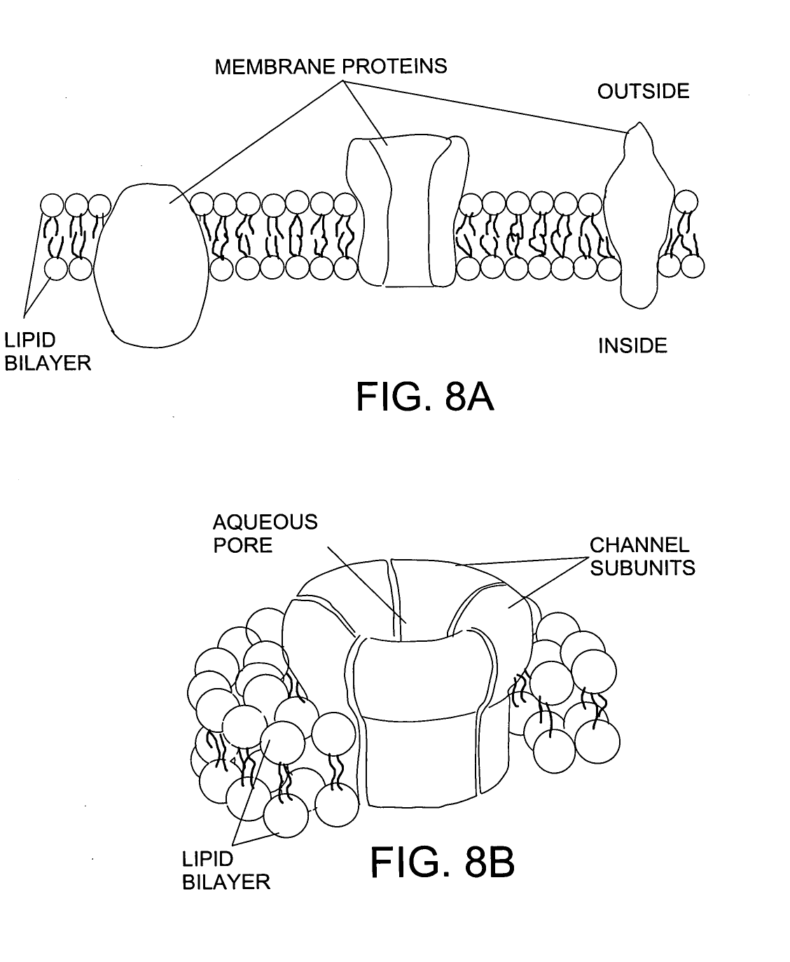

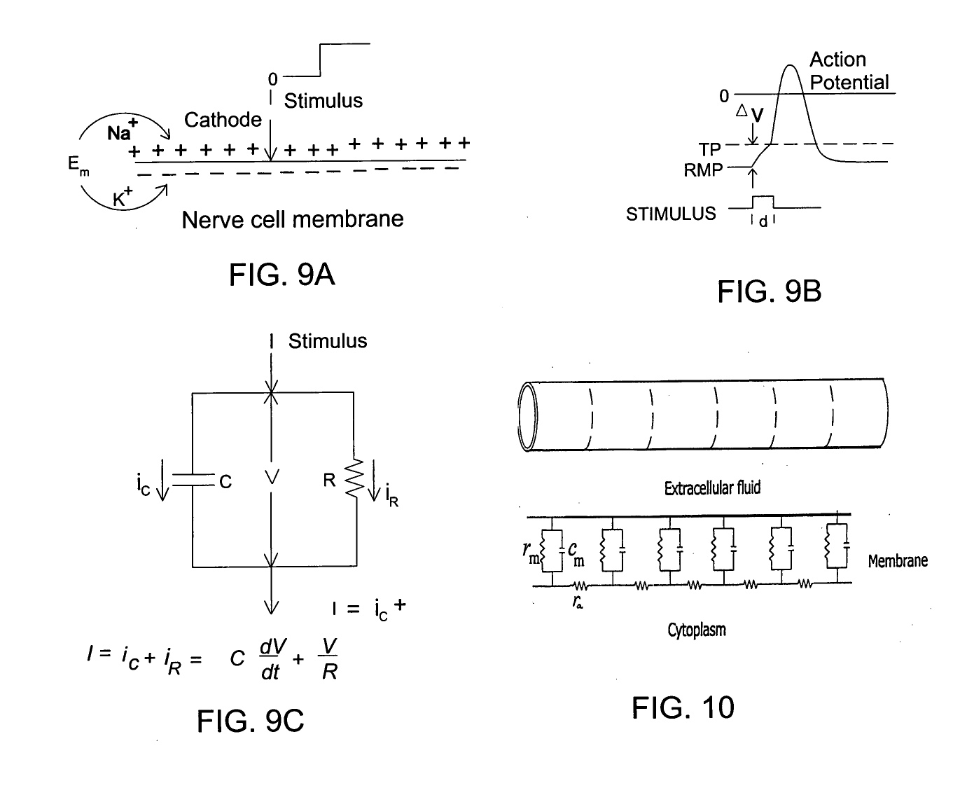

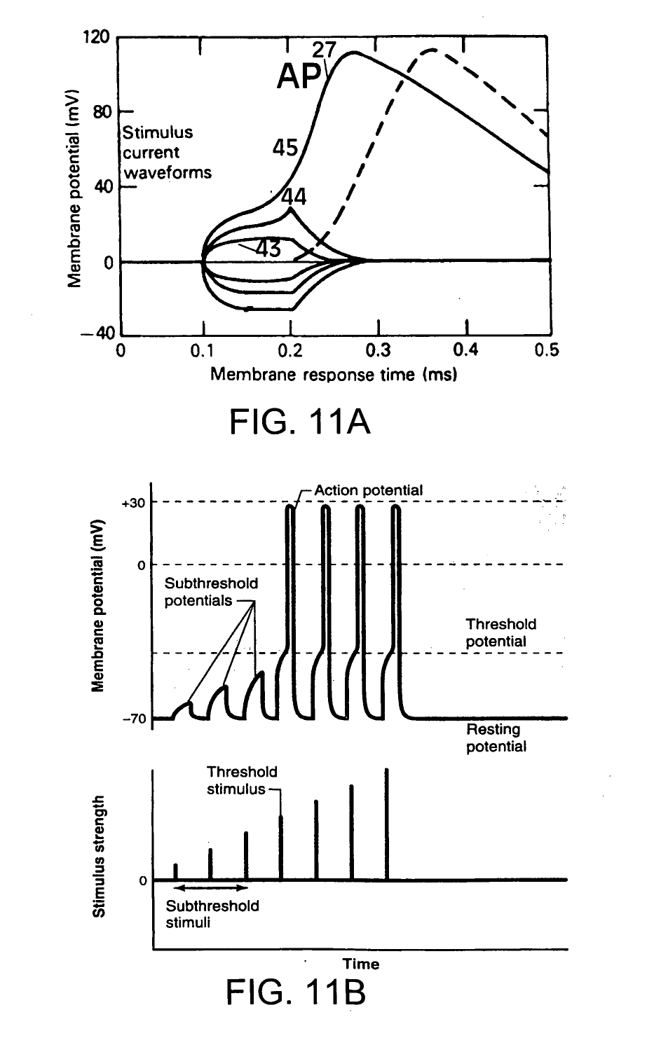

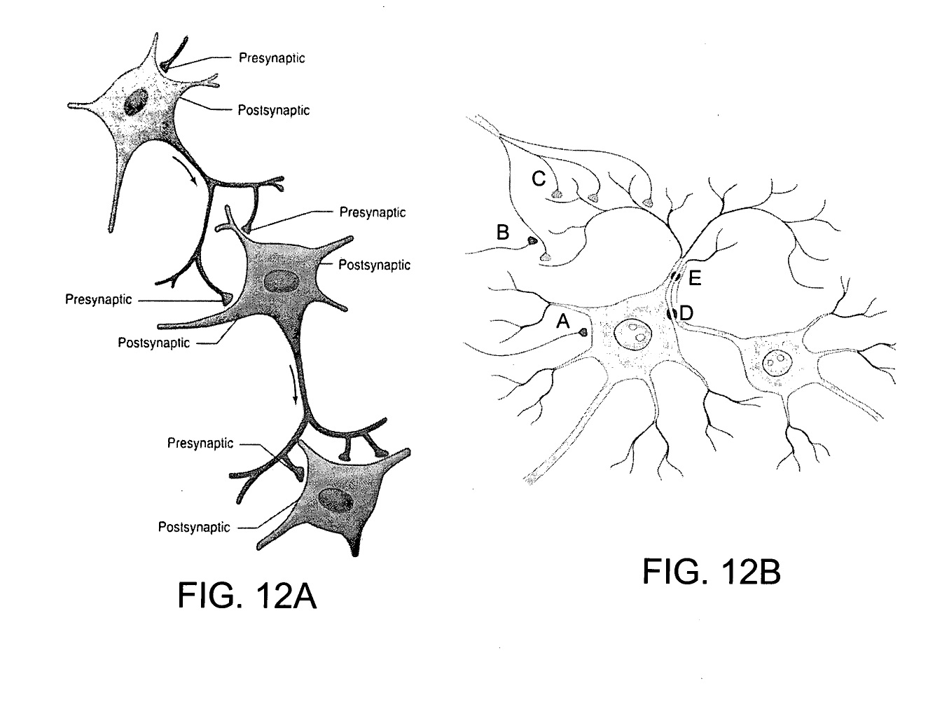

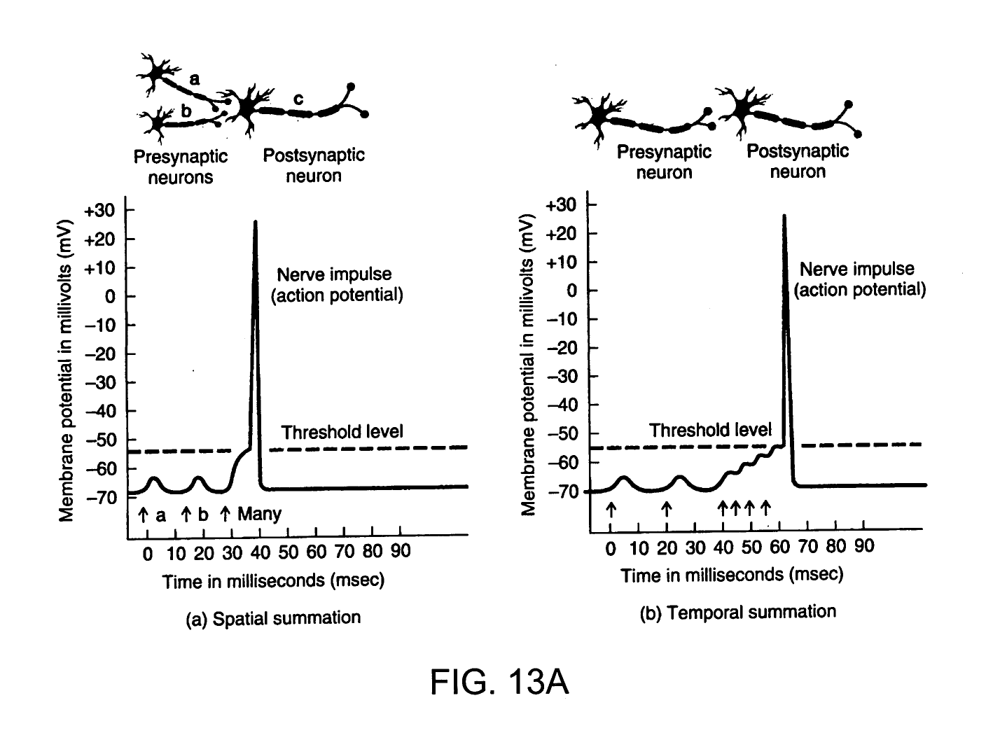

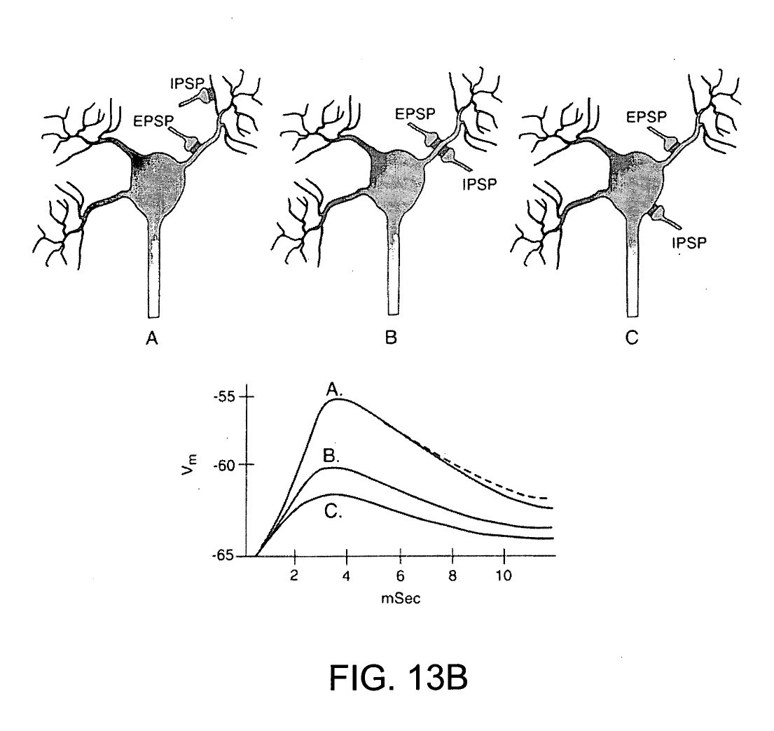

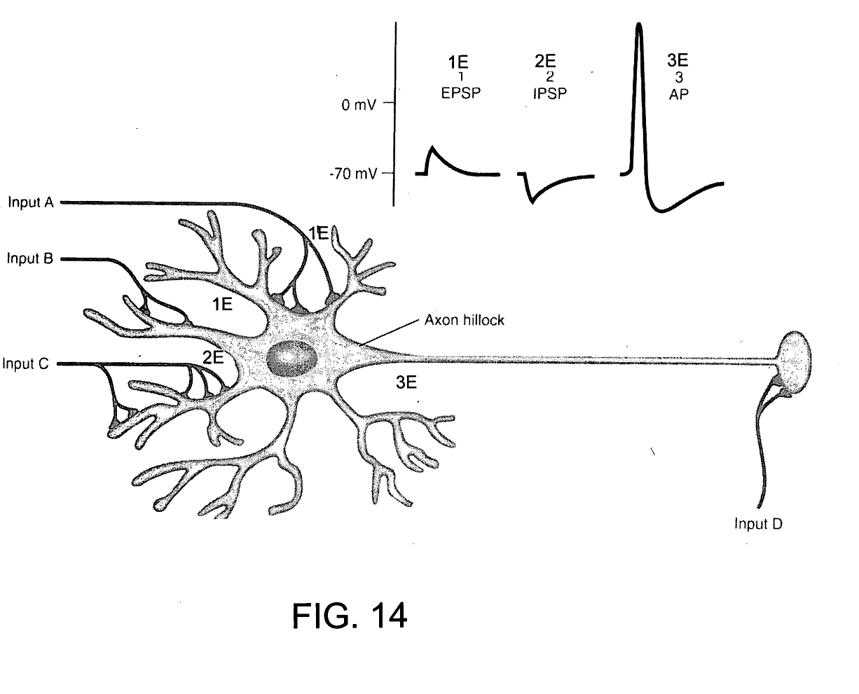

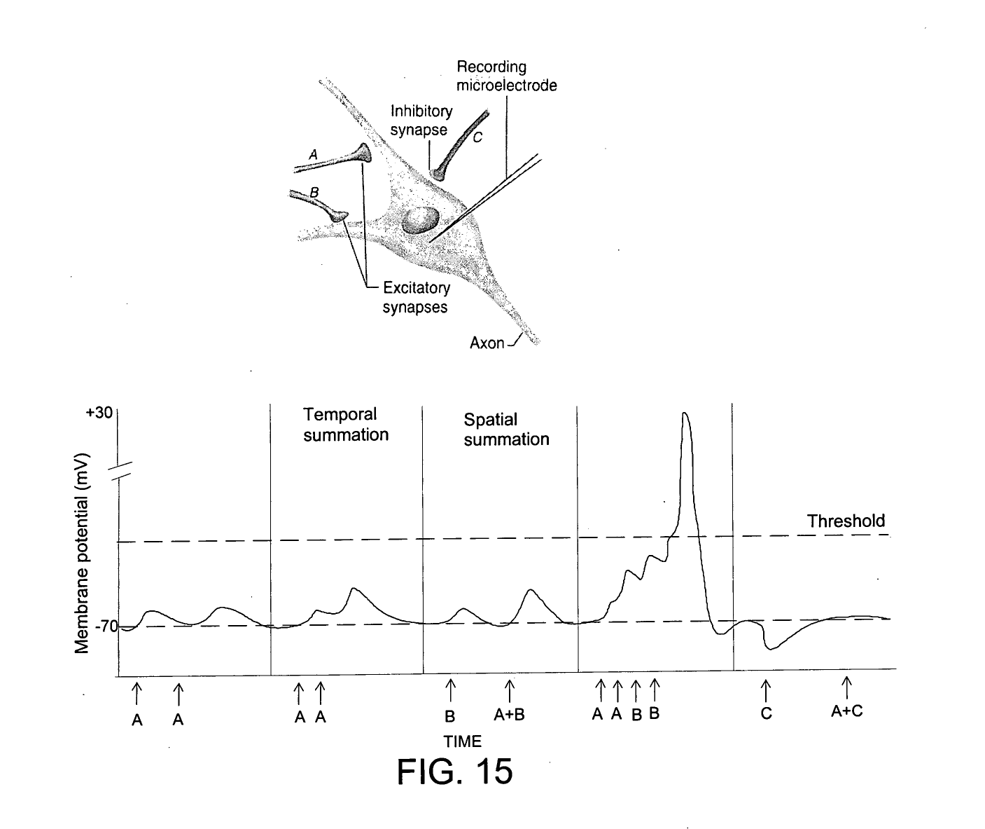

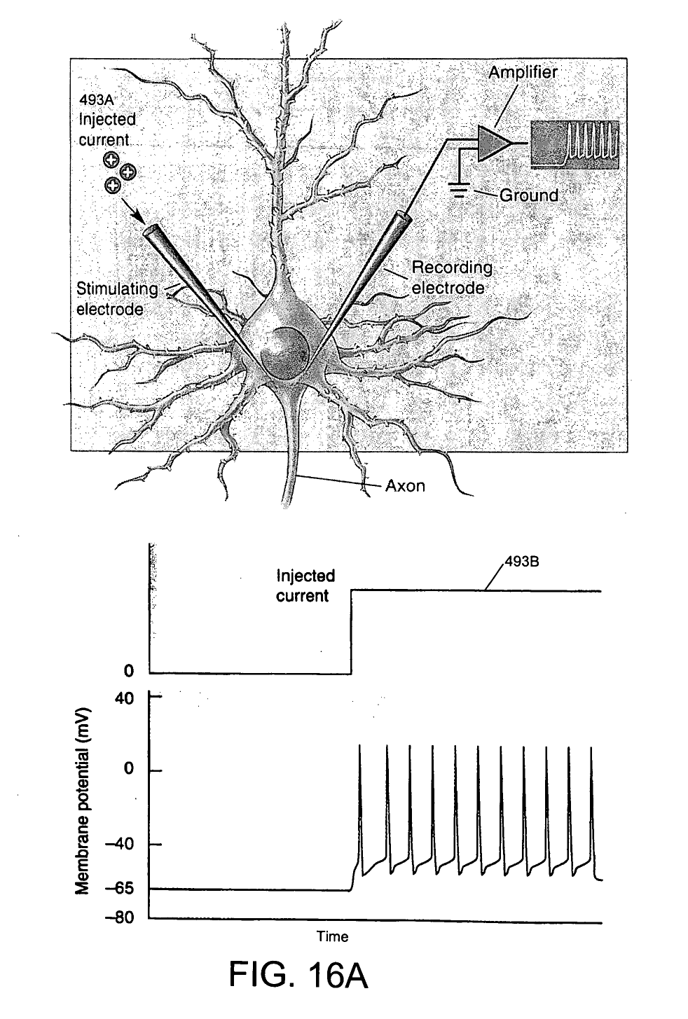

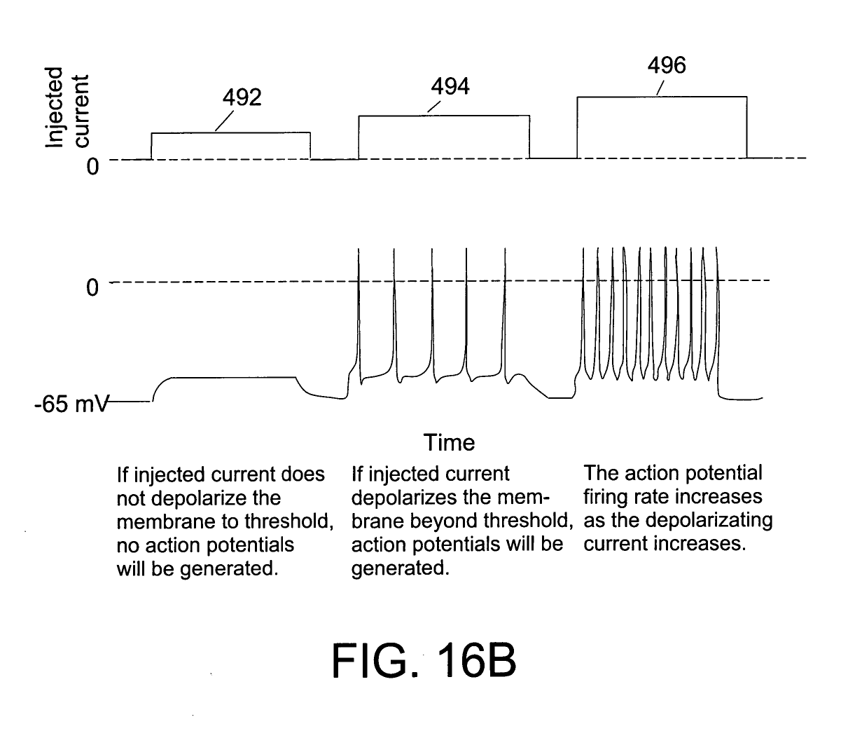

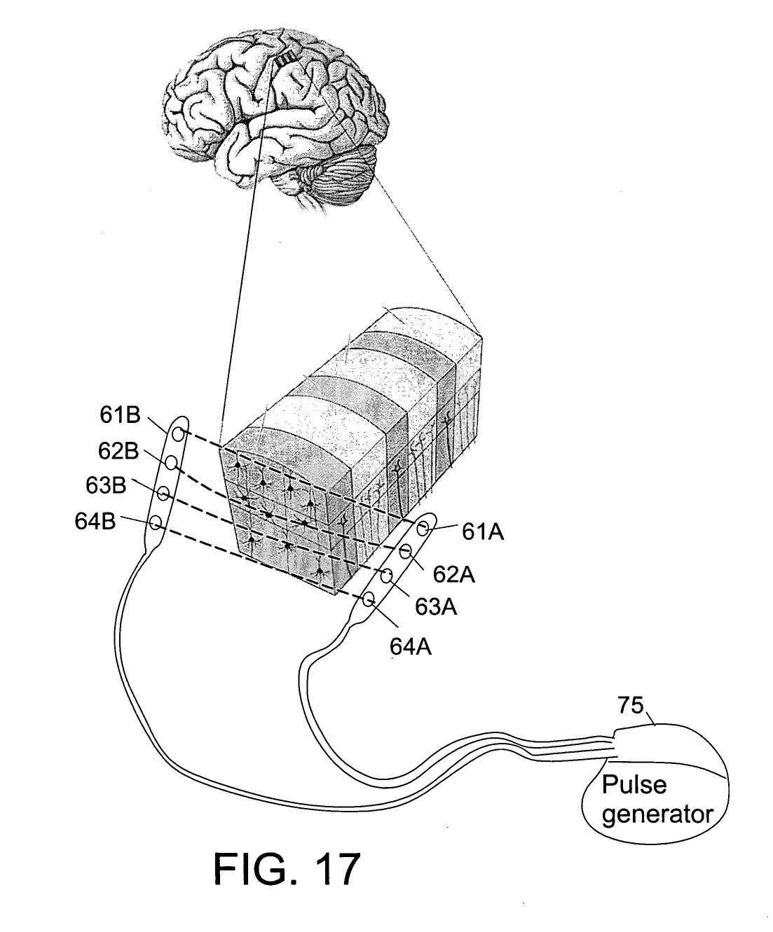

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

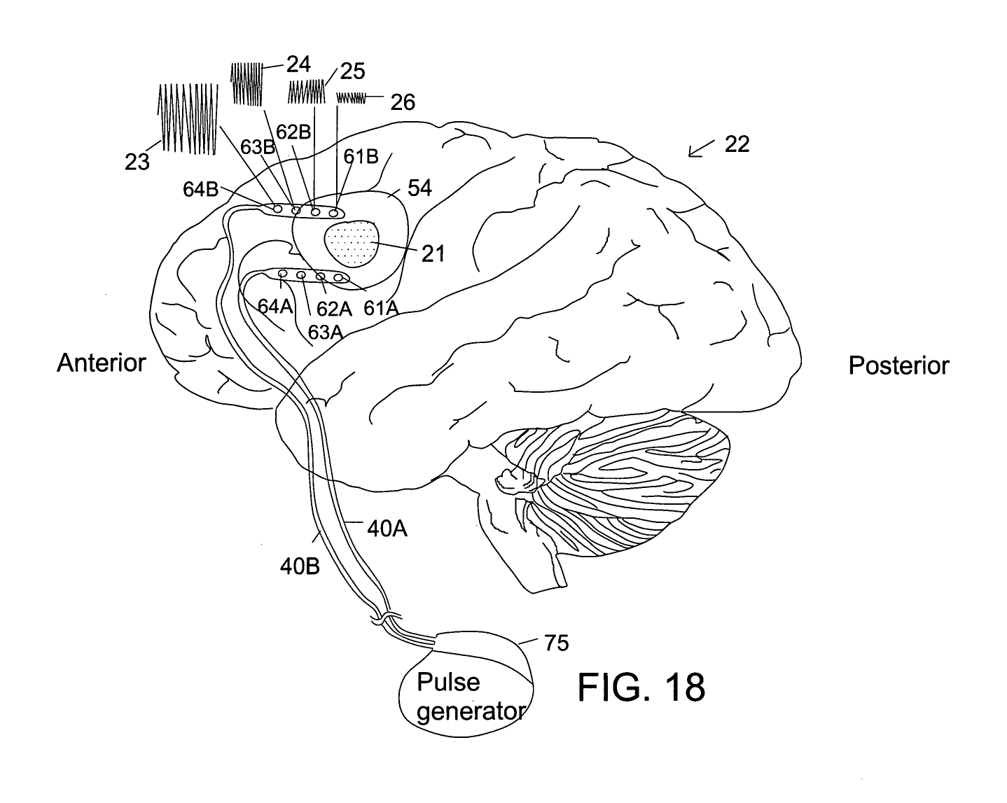

In one aspect, electrical pulses are supplied to the cortical tissue 54 based at least in part to sensing intrinsic electrical activity from the neural cortical tissue 54. This is shown in conjunction with FIG. 18, where preferably paddle electrodes (grid electrodes may also be used) are placed in relation to an ischemic region 21, such as may be caused by stroke. The paddle (or grid) electrodes are placed such that one electrode pair is closest to the ischemic tissue and one electrode pair is farthest from the ischemic tissue, and closest to “healthy” neural tissue. Based on the underlying electrophysiologic principles, it would be expected that the electrode pair 61A, 61B adjacent to the ischemic tissue 21 will record voltages 26 that are much smaller in amplitude than the voltages recorded 23 from an electrode pair 64A, 64B that is adjacent to “healthy” neural tissue. Further, it would be expected that the electrode pairs in-between will have sensed voltages 24, 25 that are somewhere in-between those of the “healthy” tissue and ischemic tissue. The differential between the sensed electrograms may be utilized for determining where to supply the electrical pulses for subthreshold stimulation.

In one embodiment, the sensed electrograms 23, 24, 25, 26 are telemetered out and recorded on paper or storage device, using a programmer and a wand placed on the implanted device. The physician can then make a determination regarding which electrodes are to be used for supplying electrical pulses, and program the pulse generator to supply electrical pulses accordingly.

In another embodiment, the microcontroller/microprocessor of the implanted pulse generator (IPG) may be used to determine the configuration for delivering stimulation pulses, based upon a predetermined criteria.

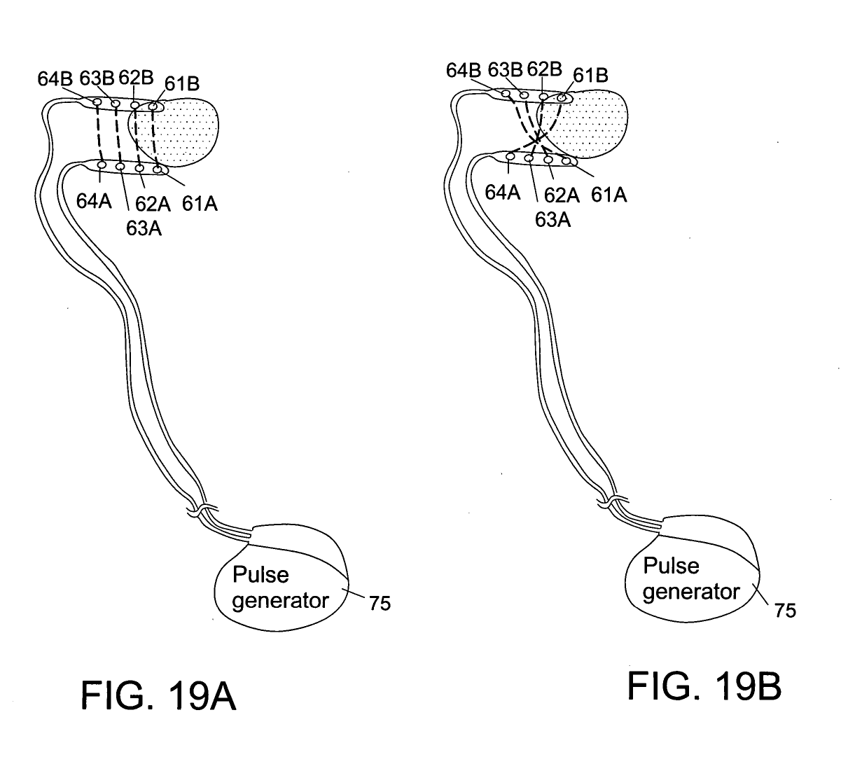

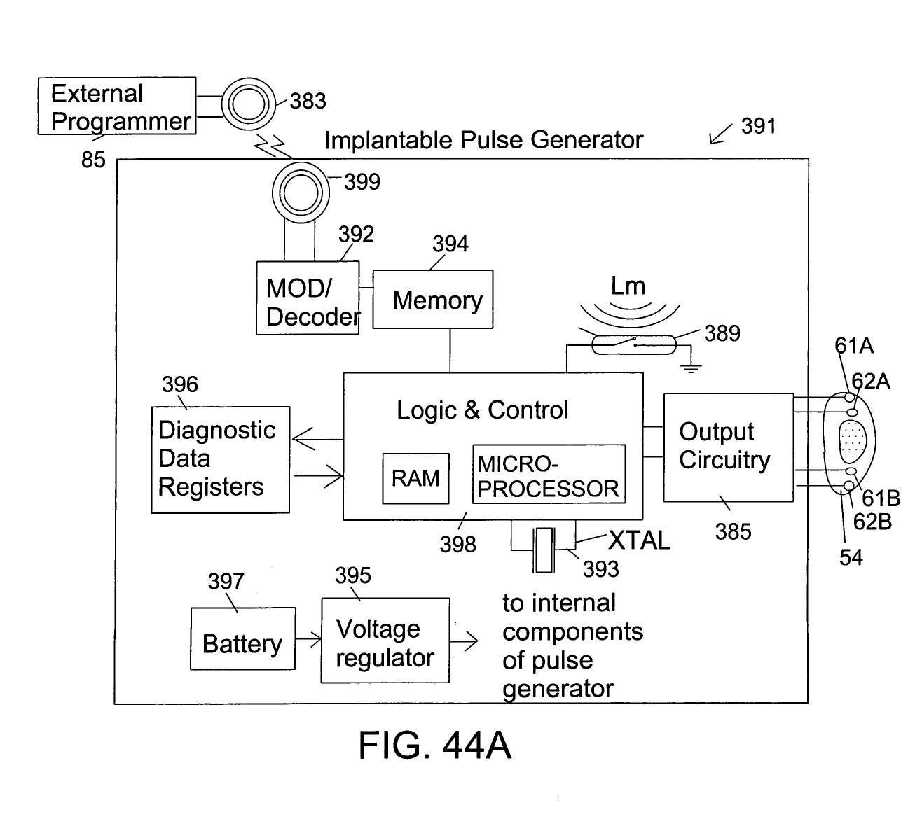

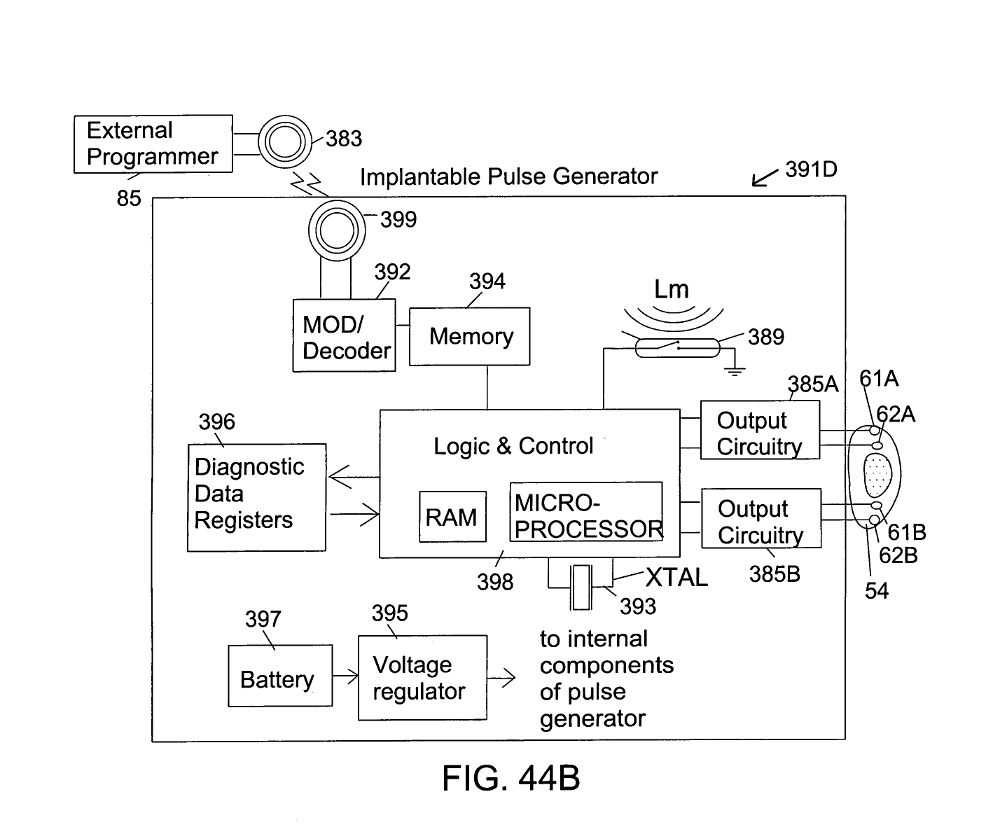

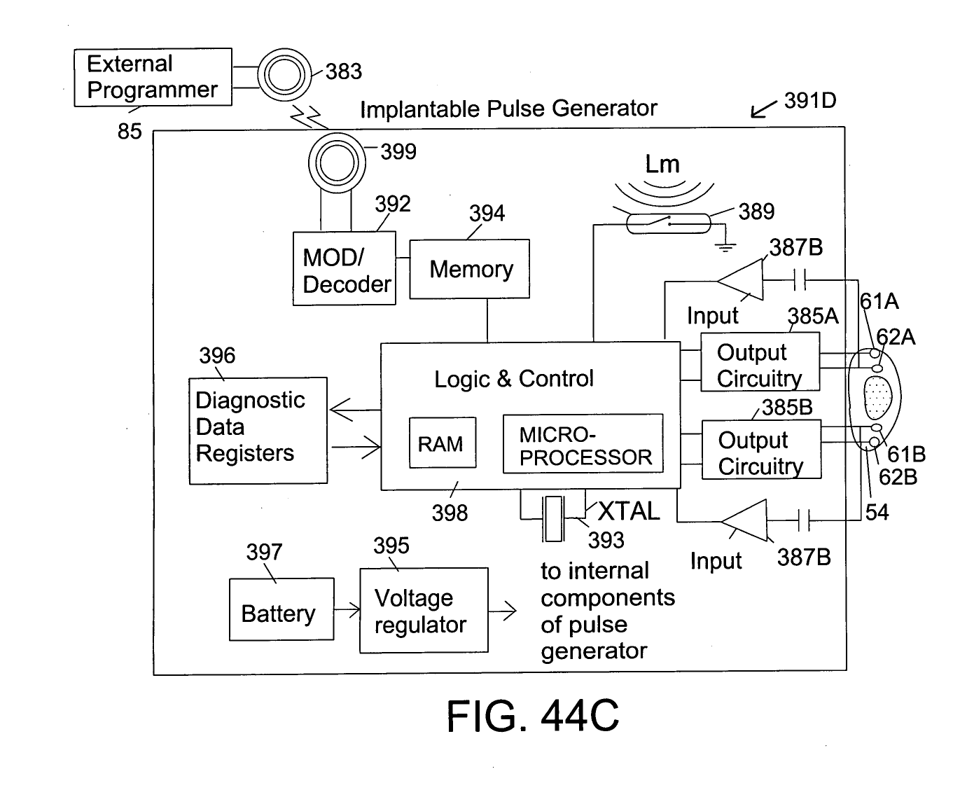

For stimulation, in one example shown in conjunction with FIGS. 19A and 19B, if the electrode pairs 61, 62, 63, 64 are used, the electrode configuration for stimulation may be 61A vs. 61B, 62A vs. 62B, 63A vs. 63B, 64A vs. 64B as shown in FIG. 19A, or the electrode stimulation configuration may be 61A vs. 64B, 62A vs. 63B, 63A vs. 62B, 64A vs. 61B, as shown in FIG. 19B, or the pulse delivery may be configured to be alternated between these two configurations. In some embodiments paddle electrodes may comprise 2, or 3, or 5 or more electrodes. Each paddle electrode may comprise more than four electrodes, and similar electrode configurations may be used for sensing or for stimulation. The object of alternating between these two configurations is to get a more even distribution of the electrical field for the tissue region being stimulated. As described later, the implantable pulse generator of this embodiment comprises sense amplifier circuitry and power source within the implanted pulse generator (FIG. 44C).

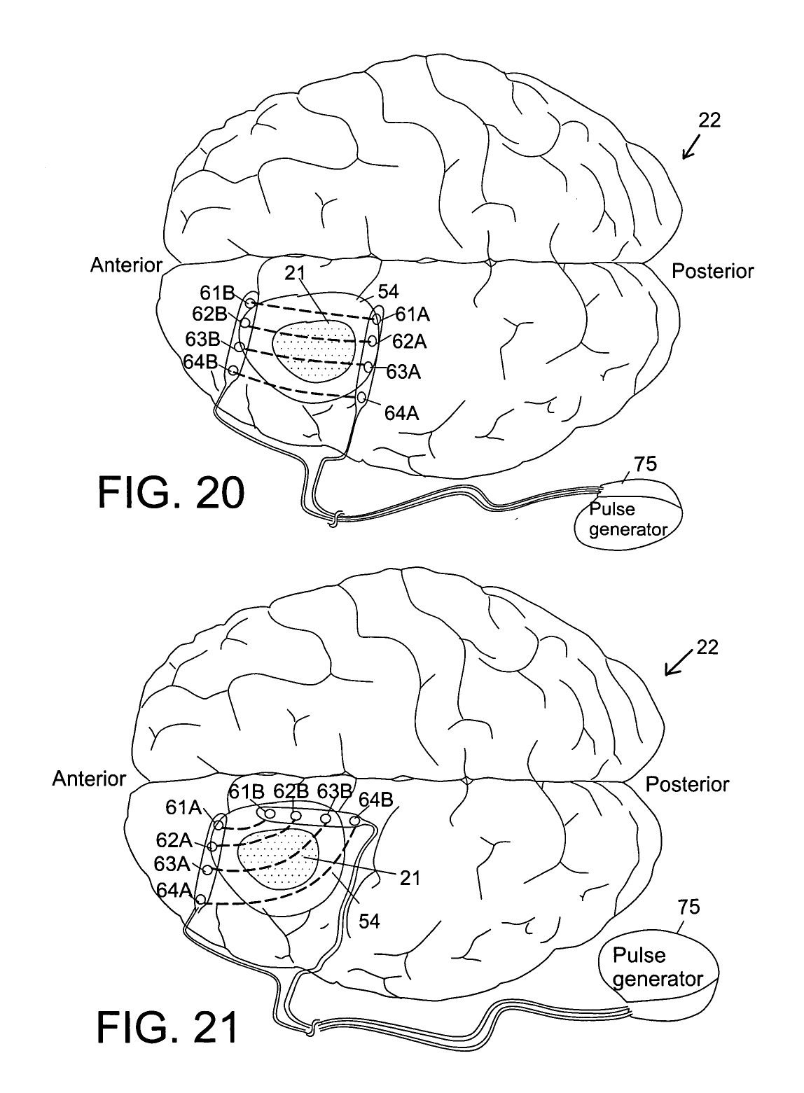

In some embodiments fMRI is used to help identify stimulation sites on the cortex of the patient. This stimulation site corresponds to the location of the brain where the intended neural activity is present. Alternatively, MRI or CT scans may be used. A grid (or paddle) electrodes are placed on the dura of the brain at the identified site. Alternatively, the grid (or paddle) electrodes may be placed subdurally. In one embodiment sensing of the brain tissue is not utilized for supplying electrical pulses. This embodiment is shown in conjunction with FIG. 20, where the paddle electrodes 61, 62, 63, 64 are placed in relation to the ischemic tissue 21 based on various imaging techniques. The stimulation configuration may be the same as shown in FIGS. 19A and 19B. That is the stimulation electrode configuration may be 61A vs. 61B, 62A vs. 62B, 63A vs. 63B, 64A vs. 64B as shown in FIG. 19A, or the electrode stimulation configuration may be 61A vs. 64B, 62A vs. 63B, 63A vs. 62B, 64A vs. 61B, as shown in FIG. 19B, or the pulse delivery may be configured to be alternated between these two configurations. Again, the object of alternating between these two configurations is to get a more even distribution of the electrical field for the tissue region being stimulated.

In some embodiments, the placement of paddle electrodes on the cortical surface is based on both digital imaging techniques as disclosed above, and sensing from the cortical tissues intraoperatively. In some embodiments, the initial approximate placement site is based on imaging techniques, and upon exposure of the cortical surface, the paddle electrodes are temporally placed at different locations, and recordings of the intrinsic neural activity are collected. The site with the most appropriate recording is used for implanting the intracranial electrodes.

The intent of the stimulation pulses is to supply a relatively even electrical field to the location where neuroplasticity is likely to be occurring, whereby neuroplasticity would be enhanced.

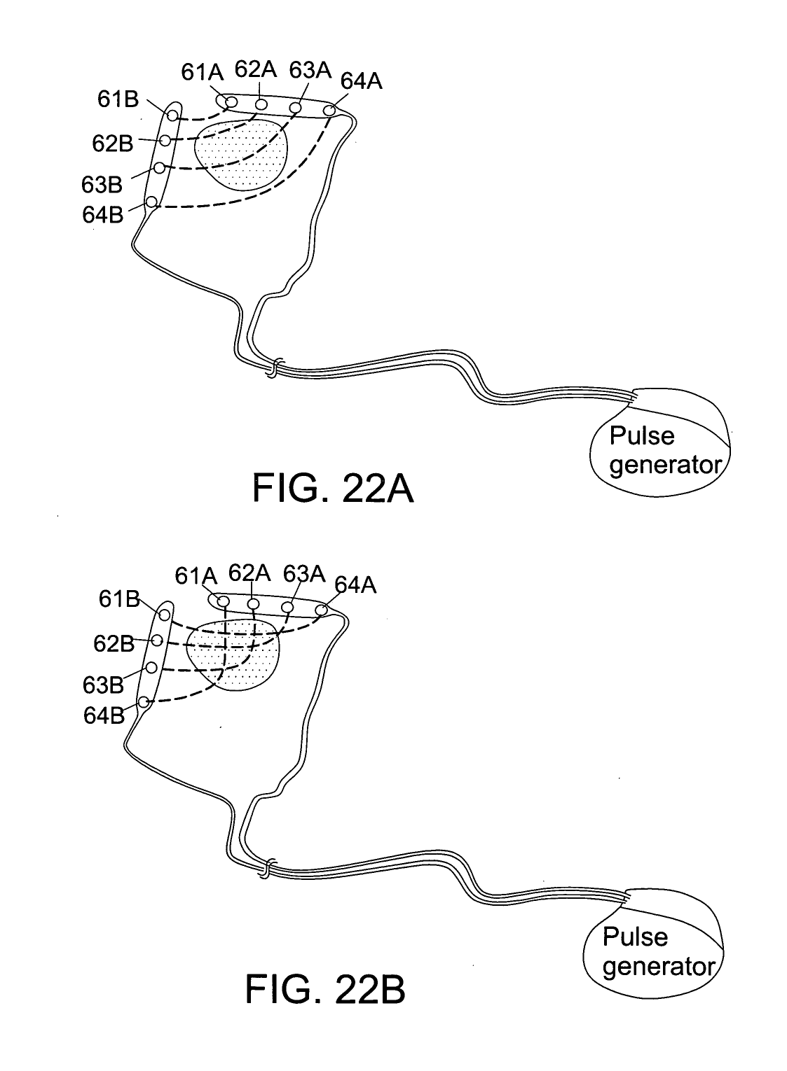

Another configuration for stimulating between two paddle electrodes is shown in conjunction with FIG. 21, where the two paddle electrodes are placed perpendicular to each other. The electrode configuration and electrical field may be as shown in conjunction with FIGS. 22A and 22B. Similar to previous examples, for stimulation the electrode configuration may be 61A vs. 61B, 62A vs. 62B, 63A vs. 63B, 64A vs. 64B as shown in FIG. 22A, or the electrode stimulation configuration may be 61A vs. 64B, 62A vs. 63B, 63A vs. 62B, 64A vs. 61B, as shown in FIG. 22B, or the pulse delivery may be configured to be alternated between these two configurations. Similar logic may be utilized using grid electrodes.

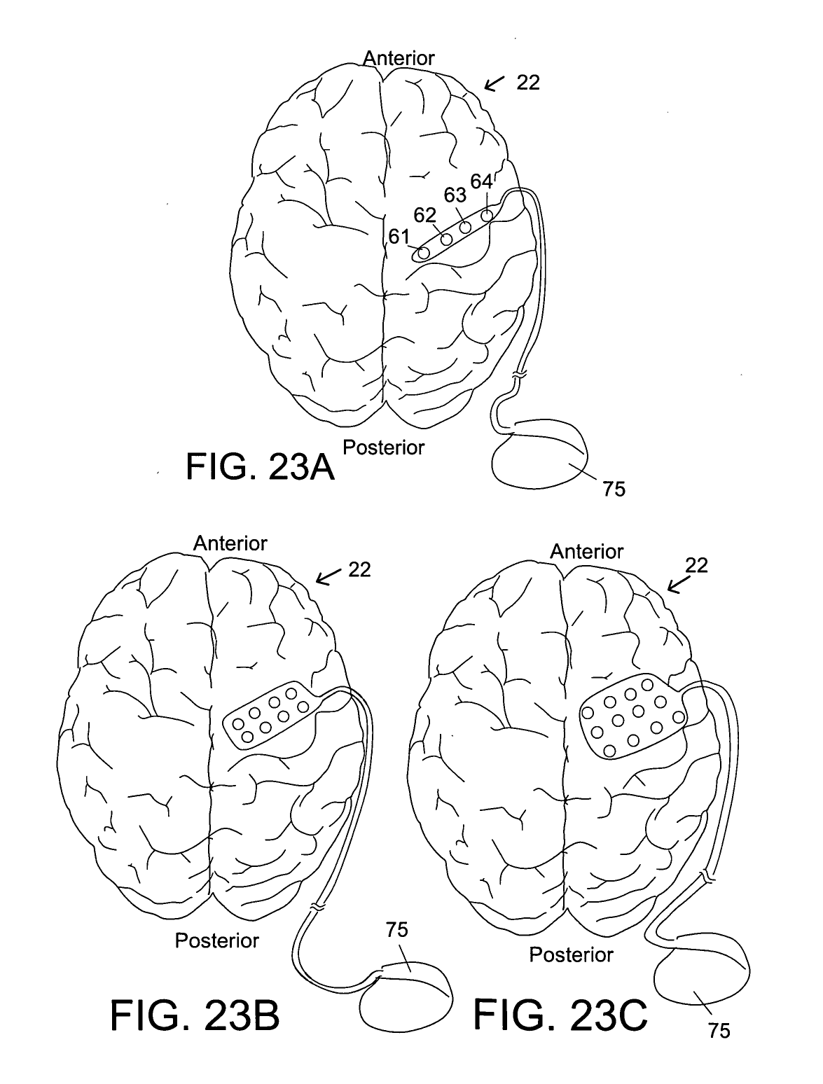

To provide therapy for other neurological disorders such as Parkinson's disease and involuntary movement disorders or other disorders, a single lead with paddle electrodes as shown in FIG. 23A, or a lead with an electrode array as shown in FIGS. 23B and 23C, may be used. Placement of the electrodes may be around the motor cortex as determined by imaging studies and/or at the discretion of the physician.

For the system to be implanted, part of the skull bone is temporarily removed to provide exposure to the brain surface, as is well known in the art. The sensing and stimulating electrodes are implanted on the surface of the brain, either on top of the dura (thick covering of the brain) or subduraly. The terminal portion of the lead is tunneled subcutaneously and connected to a pulse generator means. The pulse generator means is implanted in a convenient location either subcutaneously or submuscularly.

The pulse generator means may be one from a group comprising:

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a microstimulator;

e) a programmable implantable pulse generator;

f) a combination implantable device comprising both a stimulus-receiver and a programmable IPG; and

g) an IPG comprising a rechargeable battery.

All of these pulse generator means can generate and emit rectangular and complex electrical pulses. Complex electrical pulses comprise at least one of multi-level pulses, biphasic pulses, non-rectangular pulses, or pulses with varying amplitude during the pulse.

### Implanted Stimulus-Receiver with an External Stimulator

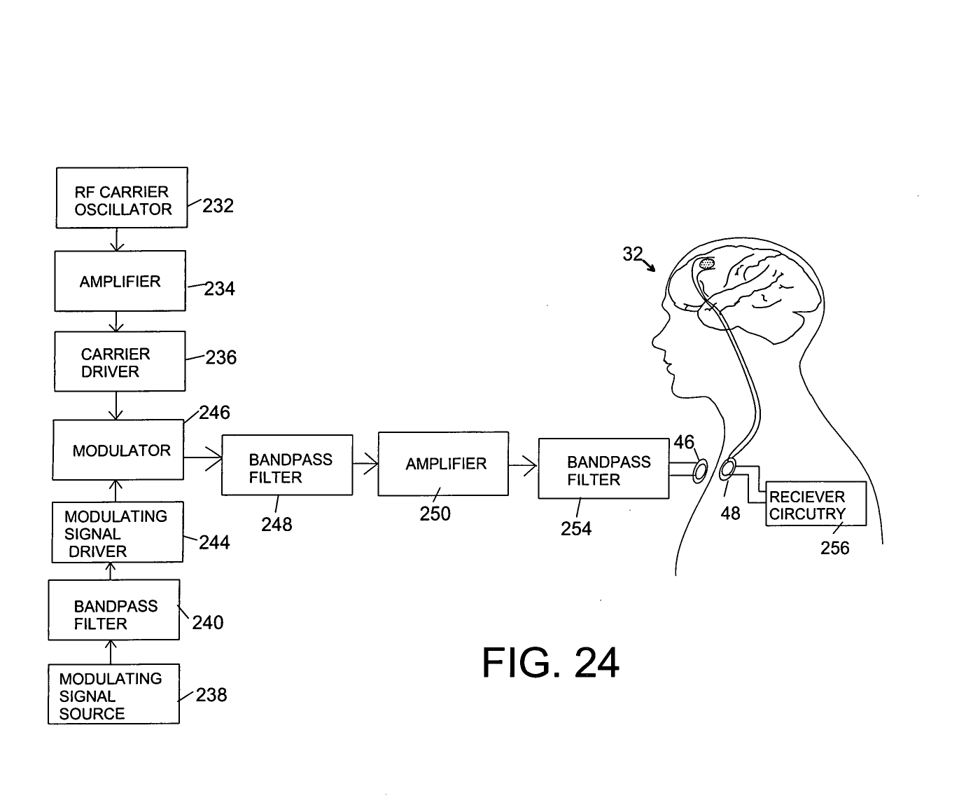

The selective stimulation of cortical brain tissues as performed by some embodiments of the method and system of this invention is shown schematically in FIG. 24, as a block diagram. Implanted stimulus-receiver with an external stimulator is also disclosed in commonly owned application Ser. No. 09/178,060 now U.S. Pat. No. 6,205,359 which is incorporated herein in entirety. A modulator 246 receives analog (sine wave) high frequency “carrier” signal and modulating signal. The modulating signal can be multilevel digital, binary, or even an analog signal. In this embodiment, mostly multilevel digital type modulating signals are used. The modulated signal is amplified 250, conditioned 254, and transmitted via a primary coil 46 which is external to the body. A secondary coil 48 of an implanted stimulus-receiver, receives, demodulates, and provides these pulses to the cortical tissues 54 via electrodes 61 and 62. Some embodiments comprise more than two electrodes. The receiver circuitry 256 is described later.

The carrier frequency is optimized. One preferred embodiment utilizes electrical signals of around 1 Mega-Hertz, even though other frequencies can be used. Low frequencies are generally not suitable because of energy requirements for longer wavelengths, whereas higher frequencies are absorbed by the tissues and are converted to heat, which again results in power losses.

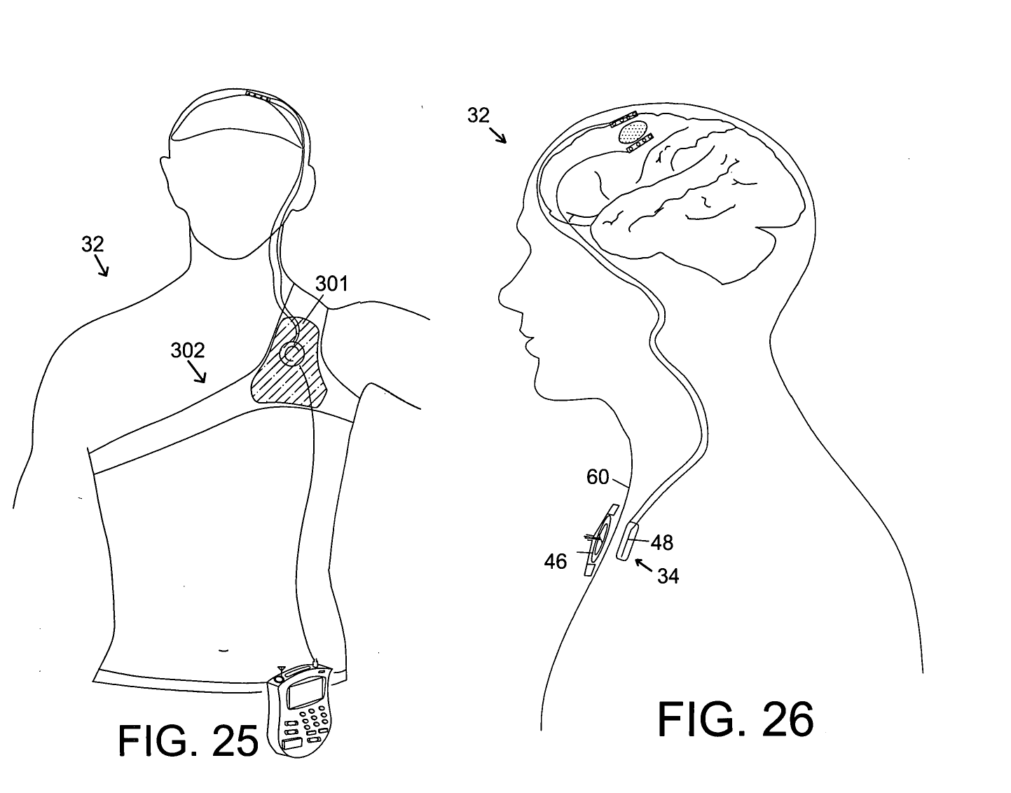

Shown in conjunction with FIG. 25, the coil for the external transmitter (primary coil 46) may be placed in the pocket 301 of a customized garment 302, for patient convenience.

Shown in conjunction with FIG. 26, the primary (external) coil 46 of the external stimulator 42 is inductively coupled to the secondary (implanted) coil 48 of the implanted stimulus-receiver 34. The implantable stimulus-receiver 34 has circuitry at the proximal end, and has two stimulating electrodes at the distal end 61,62. Even though, the implantable stimulus-receiver 34 shown here is configured to have two electrodes, the implantable stimulus-receiver 34 may also be configured to have more than two electrodes.

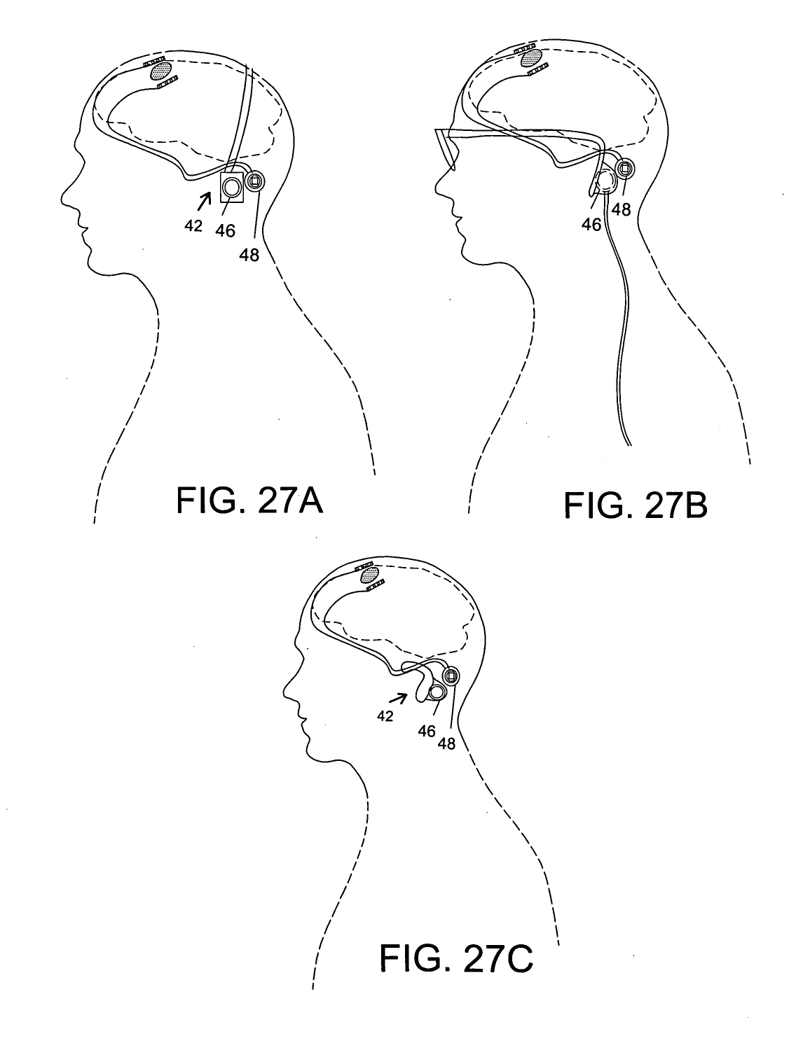

In one embodiment, the implanted stimulus-receiver comprising the implanted (secondary) coil is tunneled subcutaneously, and implanted approximately in the region behind the ear. This embodiment is shown in conjunction with FIGS. 27A, 27B, and 27C. As known in the art, the primary (external) coil 46 needs to be approximately adjacent to secondary (implanted) coil 48. In one embodiment, as shown in conjunction with FIG. 27A, a head band may be used as an aid for placing the primary (external) coil 46 in proximity with the secondary (implanted) coil 48, which is implanted subcutaneously approximately in the area behind the ear.

In another embodiment, as shown in conjunction with FIG. 27B, the primary (external) coil 46 may be placed in proximity to the secondary (implanted) coil 48 with the aid of eyeglasses. In yet another embodiment, as shown in conjunction with FIG. 27, the external stimulator may be miniaturized, and adapted to be placed around the ear similar to a hearing aid, such that the primary (external) coil 46 is conveniently positioned in proximity to the secondary (implanted) coil 48.

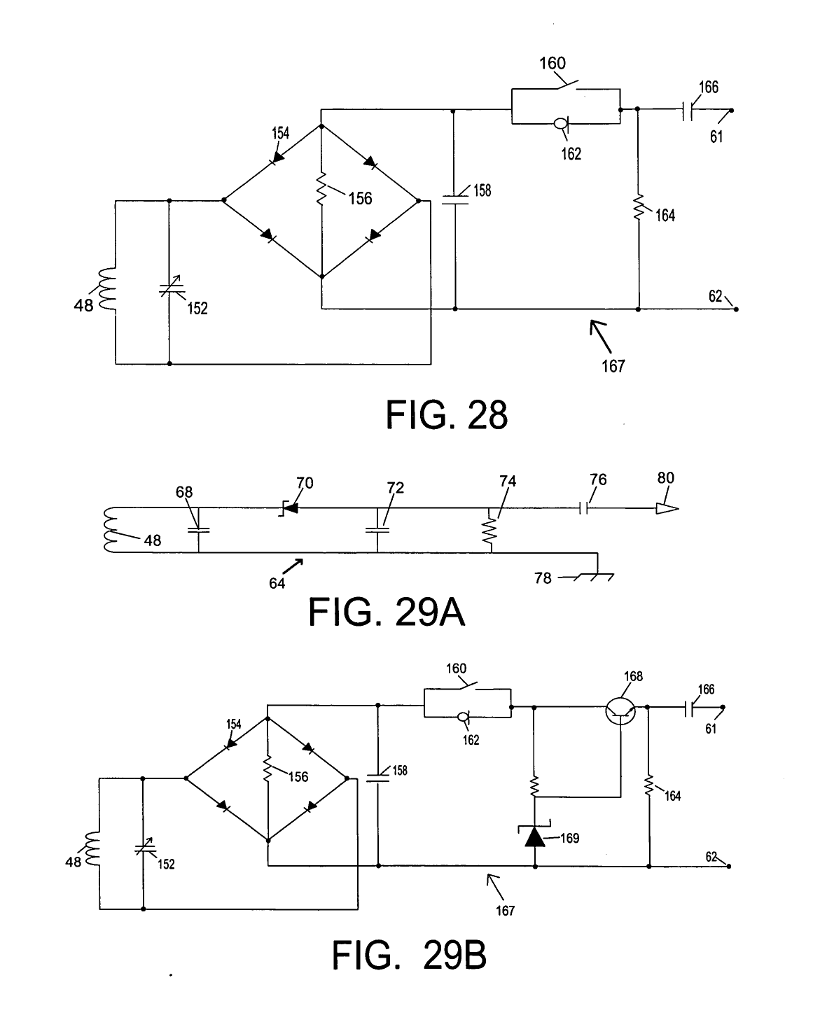

For the stimulus-receiver 34 with two electrode configuration, the circuitry contained in the proximal end of the implantable stimulus-receiver 34 is shown schematically in FIG. 28, for one embodiment. In this embodiment, the circuit uses all passive components. Approximately 25 turn copper wire of 30 gauge, or comparable thickness, is used for the primary coil 46 and secondary coil 48. This wire is concentrically wound with the windings all in one plane. The frequency of the pulse-waveform delivered to the implanted coil 48 can vary, and so a variable capacitor 152 provides ability to tune secondary implanted circuit 167 to the signal from the primary coil 46. The pulse signal from secondary (implanted) coil 48 is rectified by the diode bridge 154 and frequency reduction obtained by capacitor 158 and resistor 164. The last component in line is capacitor 166, used for isolating the output signal from the electrode wire. The return path of signal from cathode 61 will be through anode 62 placed in proximity to the cathode 61 for “Bipolar” stimulation. In this embodiment bipolar mode of stimulation is used, however, the return path can be connected to the remote ground connection (case) of implantable circuit 167, providing for much larger intermediate tissue for “Unipolar” stimulation. The “Bipolar” stimulation offers localized stimulation of tissue compared to “Unipolar” stimulation and is therefore, preferred in this embodiment. Unipolar stimulation is more likely to stimulate skeletal muscle in addition to nerve stimulation. The implanted circuit 167 in this embodiment is passive, so a battery does not have to be implanted.

The circuitry shown in FIGS. 29A and 29B can be used as an alternative, for the implanted stimulus-receiver. The circuitry of FIG. 29A is a slightly simpler version, and circuitry of FIG. 29B contains a conventional NPN transistor 168 connected in an emitter-follower configuration.

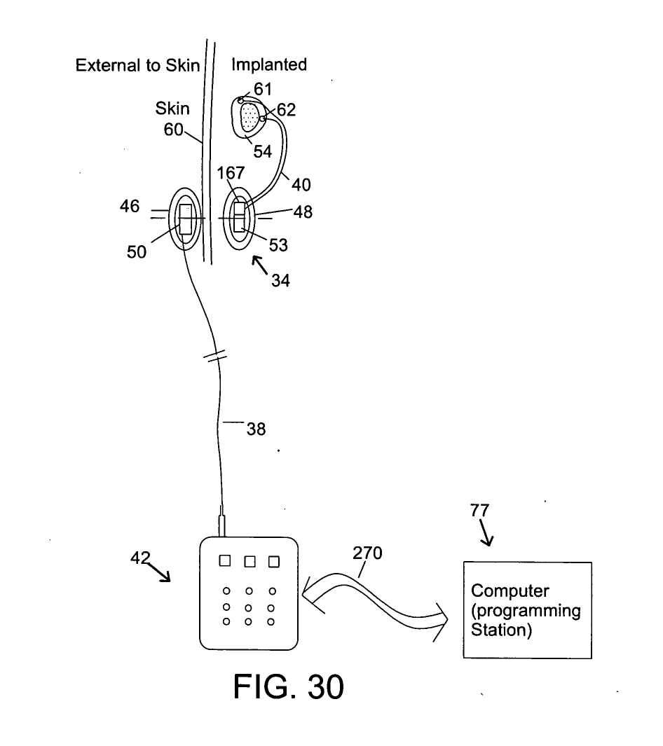

For stimulation therapy to commence, the primary (external) coil 46 is placed on the skin 60 on top of the surgically implanted (secondary) coil 48. An adhesive tape on the skin 60 may be used to hold primary (external) coil 46 in the appropriate position. For efficient energy transfer to occur, it is important that the primary (external) and secondary (internal) coils 46,48 be positioned along the same axis and be optimally positioned relative to each other. In this embodiment, the external coil 46 may be connected to proximity sensing circuitry 50. The correct positioning of the external coil 46 with respect to the internal coil 48 is indicated by turning “on” of a light emitting diode (LED) on the external stimulator 42 (FIG. 30).

Optimal placement of the external (primary) coil 46 is done with the aid of proximity sensing circuitry incorporated in the system, in this embodiment. Proximity sensing occurs utilizing a combination of external and implantable components. The implanted components contains a relatively small magnet composed of materials that exhibit Giant Magneto-Resistor (GMR) characteristics such as Samarium-cobalt, a coil, and passive circuitry. Shown in conjunction with FIG. 30, the external coil 46 and proximity sensor circuitry 50 are rigidly connected in a convenient enclosure which is attached externally on the skin. The sensors measure the direction of the field applied from the magnet to sensors within a specific range of field strength magnitude. The dual sensors exhibit accurate sensing under relatively large separation between the sensor and the target magnet. As the external coil 46 placement is “fine tuned”, the condition where the external (primary) coil 46 comes in optimal position, i.e. is located adjacent and parallel to the subcutaneous (secondary) coil 48, along its axis, is recorded and indicated by a light emitting diode (LED) on the external stimulator 42.

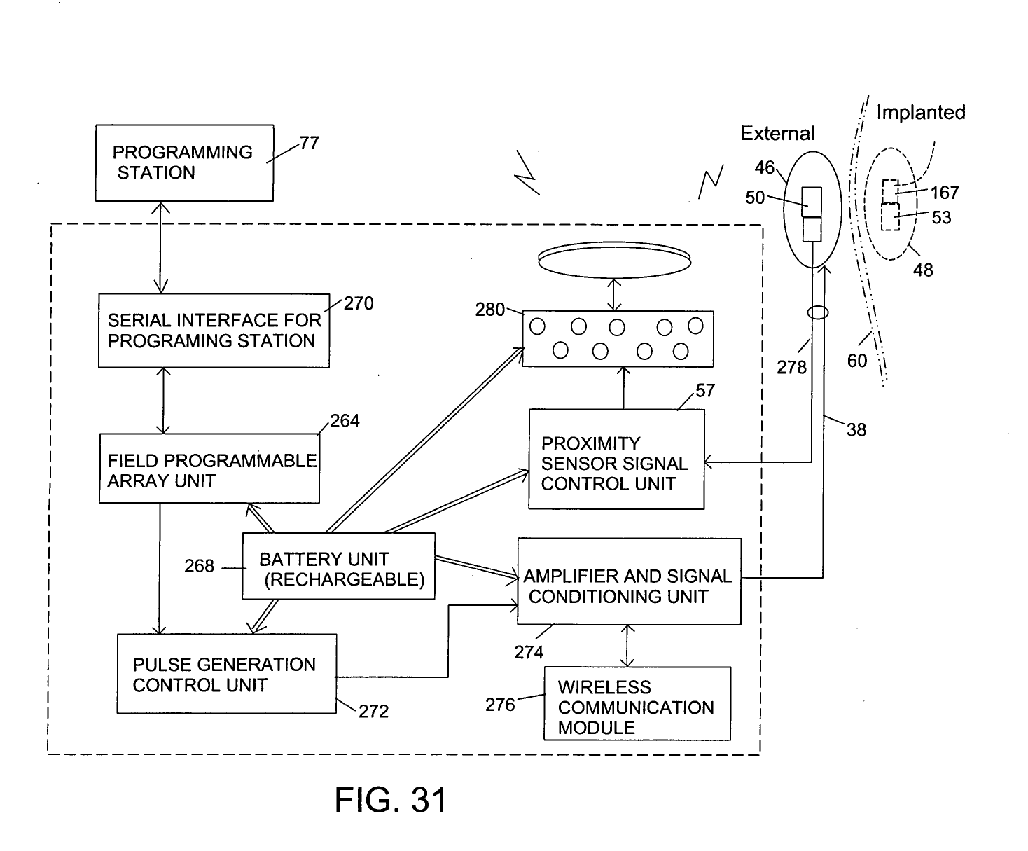

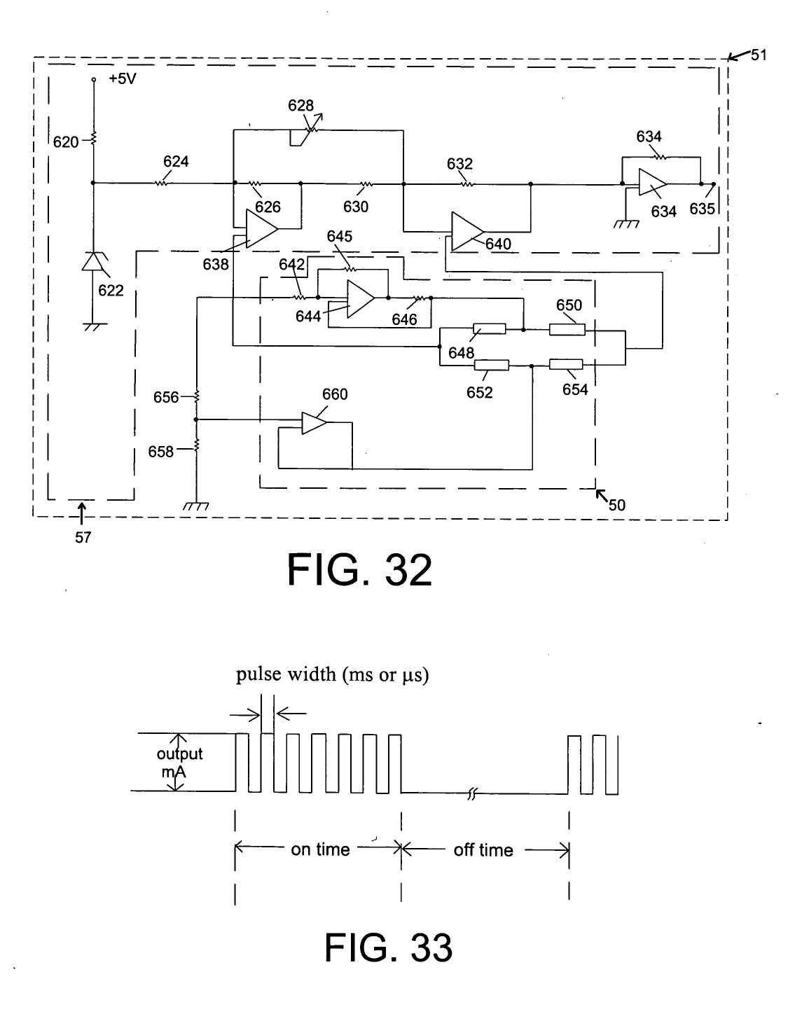

FIG. 31 shows an overall block diagram of the components of the external stimulator 42 and the proximity sensing mechanism. The proximity sensing components are the primary (external) coil 46, supercutaneous (external) proximity sensors 648, 652 (FIG. 32) in the proximity sensor circuit unit 50, and a subcutaneous secondary coil 48 with a Giant Magneto Resister (GMR) magnet 53 associated with the proximity sensor unit. The proximity sensor circuit 50 provides a measure of the position of the secondary implanted coil 48. The signal output from proximity sensor circuit 50 is derived from the relative location of the primary and secondary coils 46, 48. The sub-assemblies consist of the coil and the associated electronic components, that are rigidly connected to the coil.

The proximity sensors (external) contained in the proximity sensor circuit 50 detect the presence of a GMR magnet 53, composed of Samarium Cobalt, that is rigidly attached to the implanted secondary coil 48. The proximity sensors, are mounted externally as a rigid assembly and sense the actual separation between the coils, also known as the proximity distance. In the event that the distance exceeds the system limit, the signal drops off and an alarm sounds to indicate failure of the production of adequate signal in the secondary implanted circuit 167, as applied in this embodiment of the device. This signal is provided to the location indicator LED 280.

FIG. 32 shows the circuit used to drive the proximity sensors 648, 652 of the proximity sensor circuit 50. The two proximity sensors 648, 652 obtain a proximity signal based on their position with respect to the implanted GMR magnet 53. This circuit also provides temperature compensation. The sensors 648, 652 are ‘Giant Magneto Resistor’ (GMR) type sensors packaged as proximity sensor unit 50. There are two components of the complete proximity sensor circuit. One component is mounted supercutaneously 50, and the other component, the proximity sensor signal control unit 57 is within the external stimulator 42. The resistance effect depends on the combination of the soft magnetic layer of magnet 53, where the change of direction of magnetization from external source can be large, and the hard magnetic layer, where the direction of magnetization remains unchanged. The resistance of this sensor 50 varies along a straight motion through the curvature of the magnetic field. A bridge differential voltage is suitably amplified and used as the proximity signal.

The Siemens GMR B6 (Siemens Corp., Special Components Inc., New Jersey) is used for this function in one embodiment. The maximum value of the peak-to-peak signal is observed as the external magnetic field becomes strong enough, at which point the resistance increases, resulting in the increase of the field-angle between the soft magnetic and hard magnetic material. The bridge voltage also increases. In this application, the two sensors 648, 652 are oriented orthogonal to each other.

The distance between the magnet 53 and sensor 50 is not relevant as long as the magnetic field is between 5 and 15 KA/m, and provides a range of distances between the sensors 648, 652 and the magnetic material 53. The GMR sensor registers the direction of the external magnetic field. A typical magnet to induce permanent magnetic field is approximately 15 by 8 by 5 mm³, for this application and these components. The sensors 648, 652 are sensitive to temperature, such that the corresponding resistance drops as temperature increases. This effect is quite minimal until about 100° C. A full bridge circuit is used for temperature compensation, as shown in temperature compensation circuit 50 of FIG. 25. The sensors 648, 652 and a pair of resistors 650, 654 are shown as part of the bridge network for temperature compensation. It is also possible to use a full bridge network of two additional sensors in place of the resistors 650, 654.

The signal from either proximity sensor 648, 652 is rectangular if the surface of the magnetic material is normal to the sensor and is radial to the axis of a circular GMR device. This indicates a shearing motion between the sensor and the magnetic device. When the sensor is parallel to the vertical axis of this device, there is a fall off of the relatively constant signal at about 25 mm. separation. The GMR sensor combination varies its resistance according to the direction of the external magnetic field, thereby providing an absolute angle sensor. The position of the GMR magnet can be registered at any angle from 0 to 360 degrees.

In the external stimulator 42 shown in FIG. 31, an indicator unit 280 which is provided to indicate proximity distance or coil proximity failure (for situations where the patch containing the external coil 46, has been removed, or is twisted abnormally etc.). Indication is also provided to assist in the placement of the patch. In case of general failure, a red light with audible signal is provided when the signal is not reaching the subcutaneous circuit. The indicator unit 280 also displays low battery status. The information on the low battery, normal and out of power conditions forewarns the user of the requirements of any corrective actions.

Also shown in FIG. 31, the programmable parameters are stored in a programmable logic 264. The predetermined programs stored in the external stimulator 42 are capable of being modified through the use of a separate programming station 77. The Programmable Array Logic Unit 264 and interface unit 270 are interfaced to the programming station 77. The programming station 77 can be used to load new programs, change the existing predetermined programs or the program parameters for various stimulation programs. The programming station is connected to the programmable array unit 75 (comprising programmable array logic 304 and interface unit 270) with an RS232-C serial connection. The main purpose of the serial line interface is to provide an RS232-C standard interface. Other suitable connectors such as a USB connector or other connectors with standard protocols may also be used.

This method enables any portable computer with a serial interface to communicate and program the parameters for storing the various programs. The serial communication interface receives the serial data, buffers this data and converts it to a 16 bit parallel data. The programmable array logic 264 component of programmable array unit receives the parallel data bus and stores or modifies the data into a random access matrix. This array of data also contains special logic and instructions along with the actual data. These special instructions also provide an algorithm for storing, updating and retrieving the parameters from long-term memory. The programmable logic array unit 264, interfaces with long term memory to store the predetermined programs. All the previously modified programs can be stored here for access at any time, as well as, additional programs can be locked out for the patient. The programs consist of specific parameters and each unique program will be stored sequentially in long-term memory. A battery unit is present to provide power to all the components. The logic for the storage and decoding is stored in a random addressable storage matrix (RASM).

Conventional microprocessor and integrated circuits are used for the logic, control and timing circuits. Conventional bipolar transistors are used in radio-frequency oscillator, pulse amplitude ramp control and power amplifier. A standard voltage regulator is used in low-voltage detector. The hardware and software to deliver the pre-determined programs is well known to those skilled in the art.

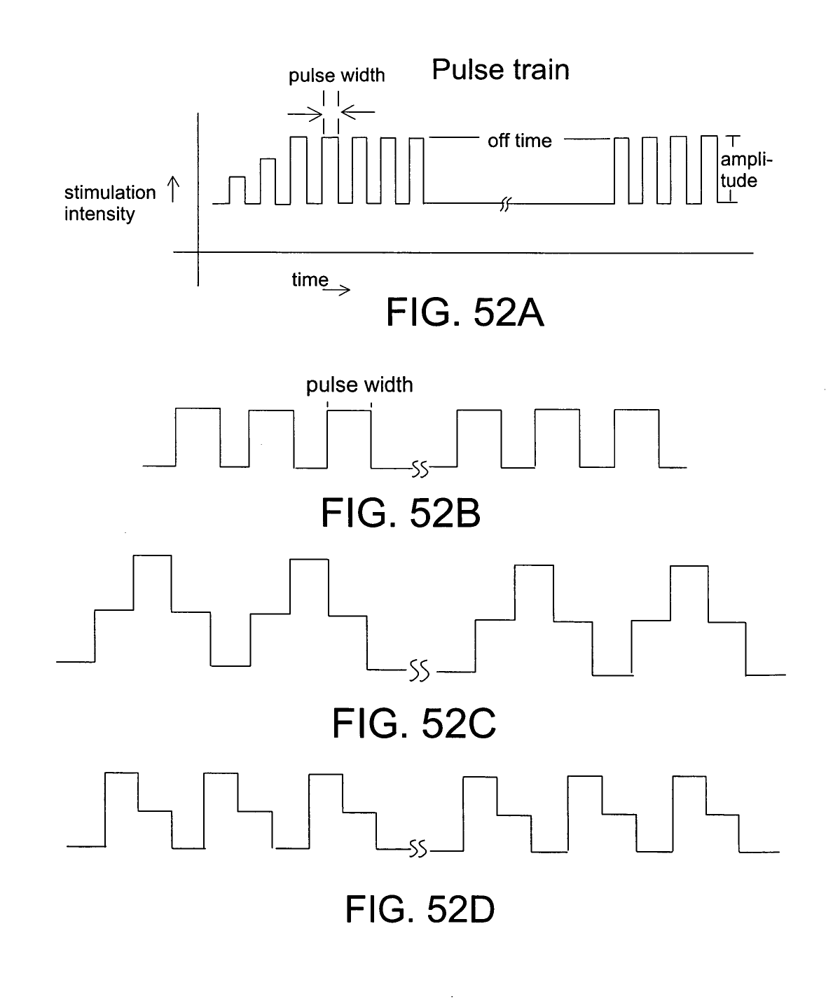

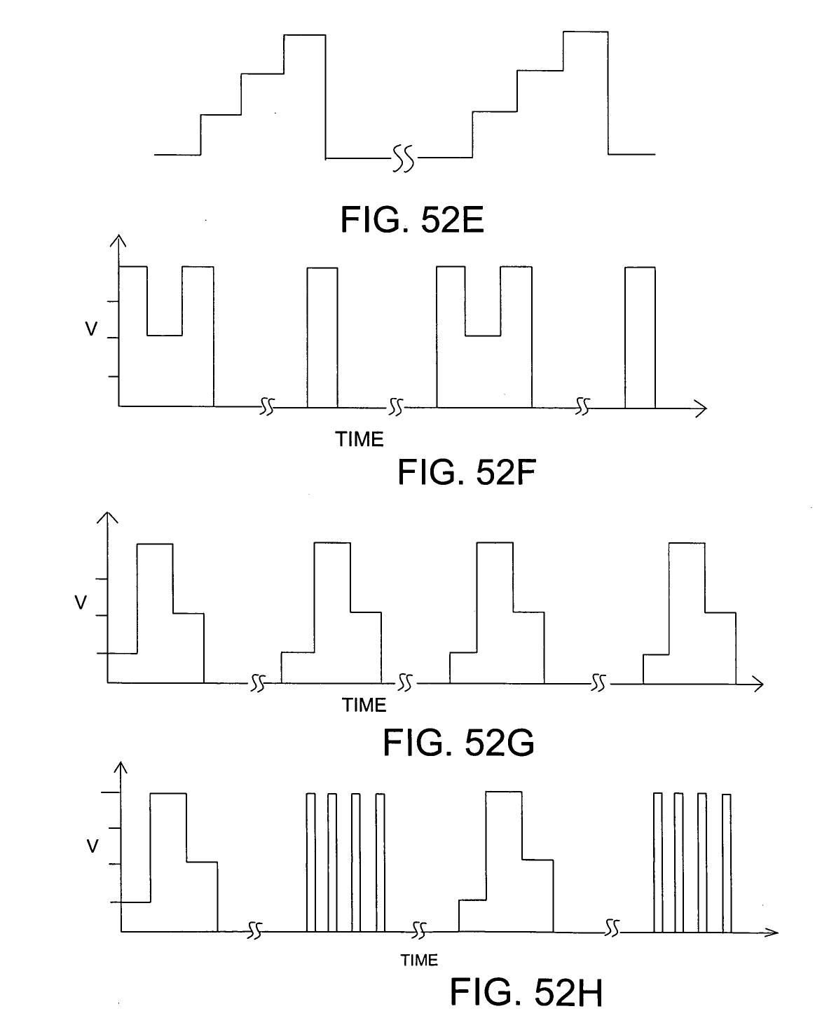

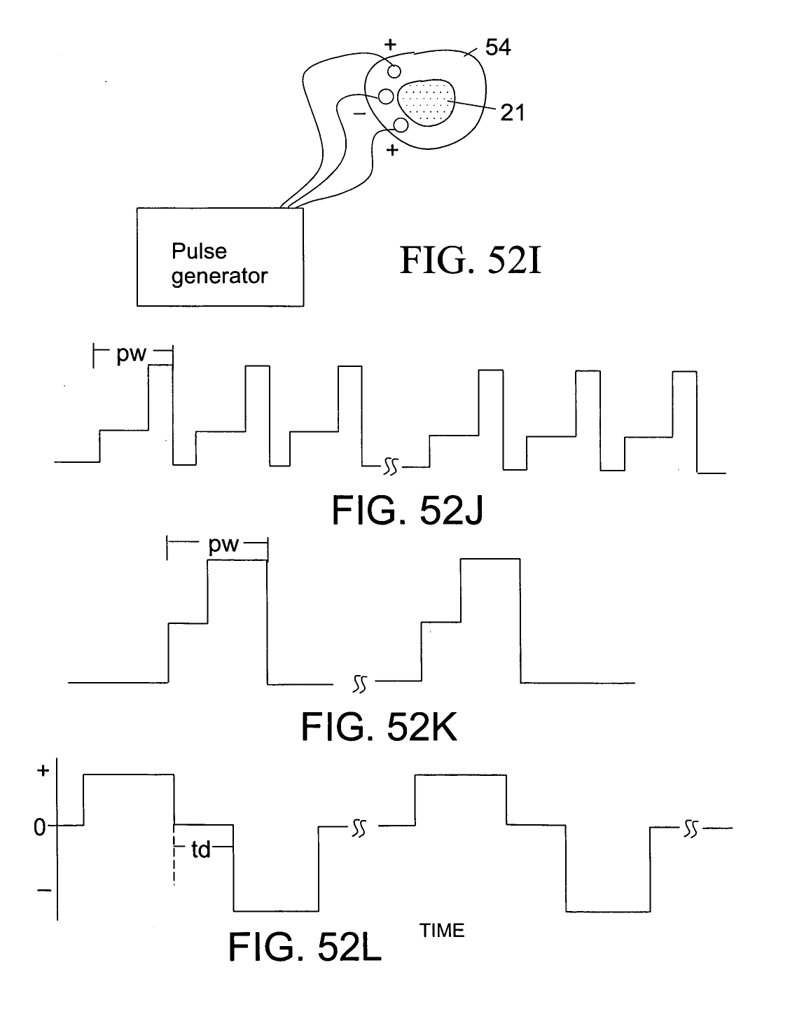

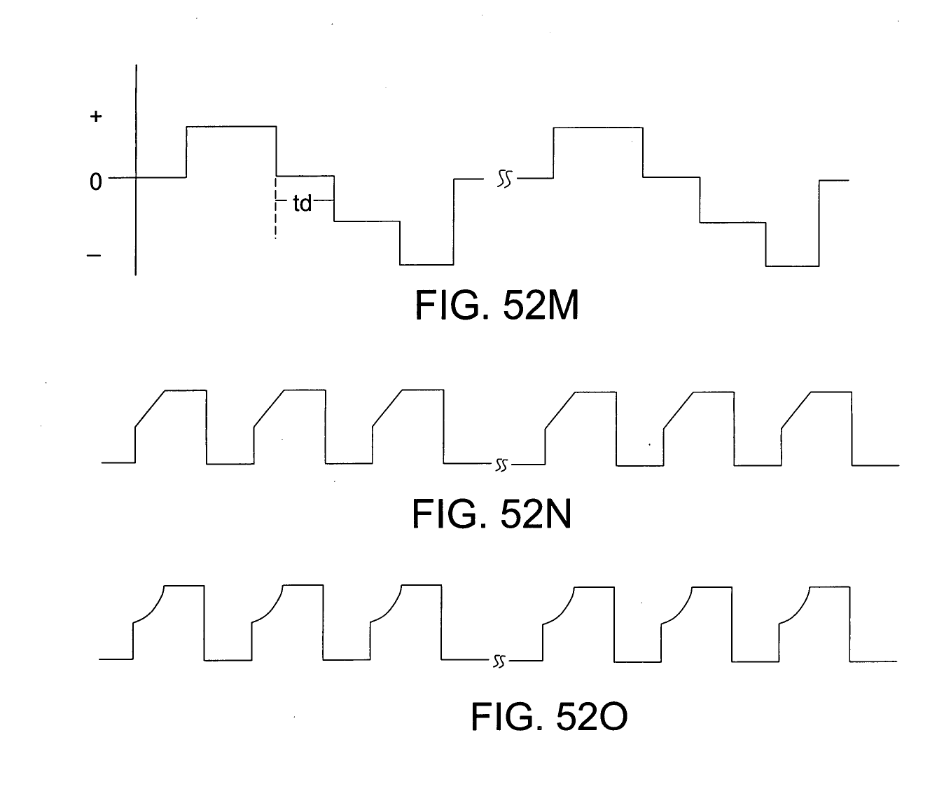

The pulses delivered to the cortical neural tissue for stimulation therapy are shown graphically in FIG. 33. In addition to rectangular pulses shown in FIG. 33, the external pulse generator may also be configured to provide complex electrical pulses. Complex electrical pulses are described later and shown in conjunction with FIGS. 52A to 52-O.

The selective stimulation of the cortical neural tissue can be performed in one of two ways. One method is to activate one of several predetermined/pre-packaged programs. A second method is to “custom” program the electrical parameters which can be selectively programmed, for specific therapy to the individual patient. The electrical parameters which can be individually programmed, include variables such as pulse amplitude, pulse width, frequency of stimulation, stimulation on-time, and stimulation off-time. Table one below defines the approximate range of parameters,

TABLE 1
Electrical parameter range delivered to the cortical tissues

| PARAMER | RANGE |
|---|---|
| Pulse Amplitude | 0.1 Volt-25 Volts |
| Pulse width | 20 μS-5 mSec. |
| Stim. Frequency | 5 Hz-150 Hz |
| Freq. for blocking | 100 to 1,000 Hz |
| On-time | 5 Secs-24 hours |
| Off-time | 5 Secs-24 hours |

The parameters in Table 1 are the electrical signals delivered to the cortical tissue via the two electrodes 61,62 (distal and proximal) around the tissue 54. It being understood that the signals generated by the external pulse generator 42 and transmitted via the primary coil 46 are larger, because the attenuation factor between the primary coil and secondary coil is approximately 10-20 times, depending upon the distance, and orientation between the two coils. Accordingly, the range of transmitted signals of the external pulse generator are approximately 10-20 times larger than shown in Table 2.

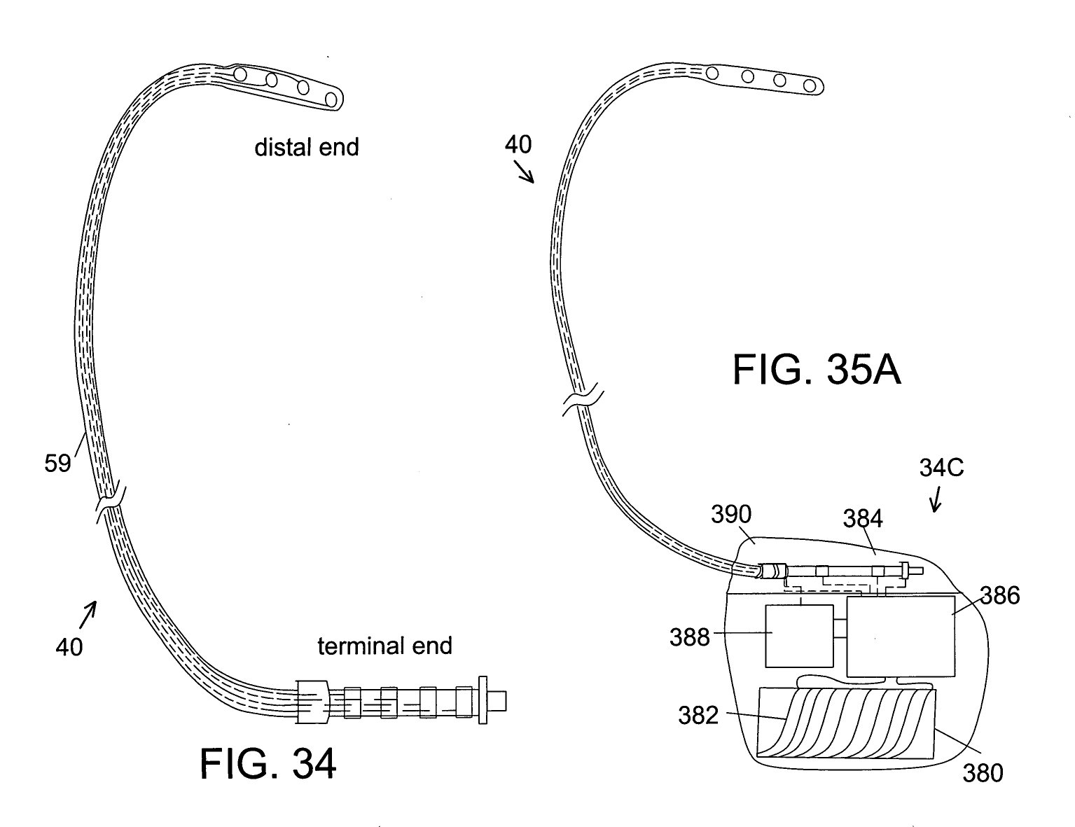

FIG. 34 depicts one example of the implanted lead 40 component of the system, showing both the proximal end and the paddle electrodes at the distal end, which may comprise between one and six electrodes. An embodiment of paddle shaped distal end comprising 4 electrodes is depicted in FIG. 34. The lead terminal preferably is linear bipolar, even though it can be bifurcated, and plug(s) into the cavity of the pulse generator means. The lead body 59 insulation may be constructed of medical grade silicone, silicone reinforced with polytetrafluoro-ethylene (PTFE), or polyurethane. The electrodes 61, 62, 63, 64 for stimulating the cortical neural tissue 54 may be “button” shaped or “pancake” shaped. These stimulating electrodes may be made of pure platinum, platinum/Iridium alloy or platinum/iridium coated with titanium nitride. The conductor connecting the terminal to the electrodes 61,62 is made of an alloy of nickel-cobalt. The implanted lead design variables are also summarized in table two below.

TABLE 2
Lead design variables

| Proximal | | | | | Distal |
|---|---|---|---|---|---|
| End | | | | | End |
| | | | Conductor | | |
| | | | (connecting | | |
| | Lead body- | | proximal | | |
| Lead | Insulation | | and distal | Electrode- | Electrode- |
| Terminal | Materials | Lead-Coating | ends) | Material | Type |
| Linear | Polyurethane | Antimicrobial | Alloy of | Pure | “Button” |
| bipolar | | coating | Nickel- | Platinum | shaped |
| | | | Cobalt | | |
| Bifurcated | Silicone | Anti- | | Platinum- | “Pancake” |
| | | Inflammatory | | Iridium | shaped |
| | | coating | | (Pt/Ir) Alloy | |
| | Silicone with | Lubricious | | Pt/Ir coated | |
| | Polytetrafluoro- | coating | | with Titanium | |
| | ethylene | | | Nitride | |
| | (PTFE) | | | | |
| | | | | Carbon | |

Once the lead is fabricated, coating such as anti-microbial, anti-inflammatory, or lubricious coating may be applied to the body of the lead.

### Implanted Stimulus-Receiver Comprising a High Value Capacitor for Storing Charge, Used in Conjunction with an External Stimulator

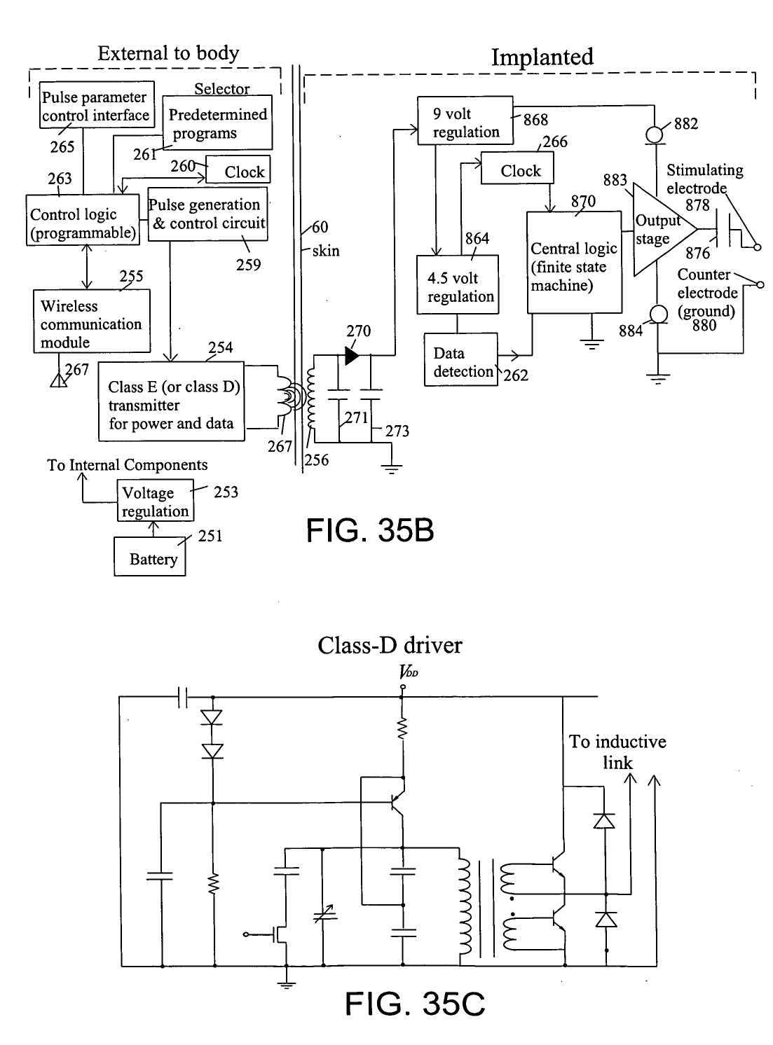

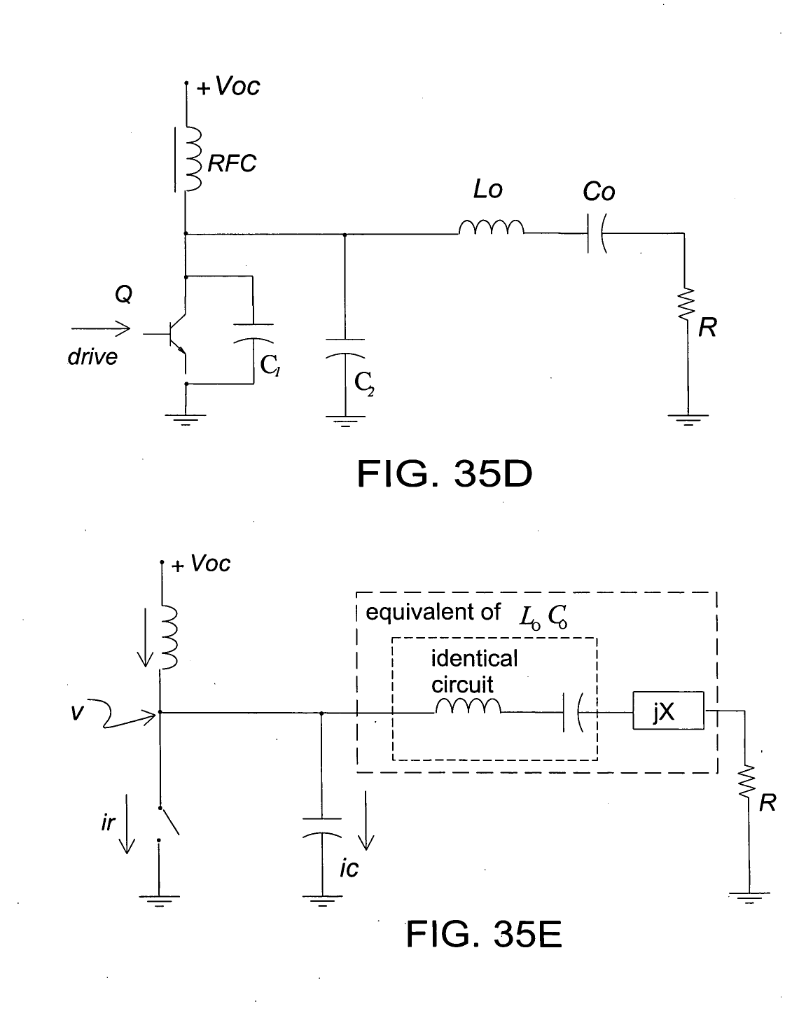

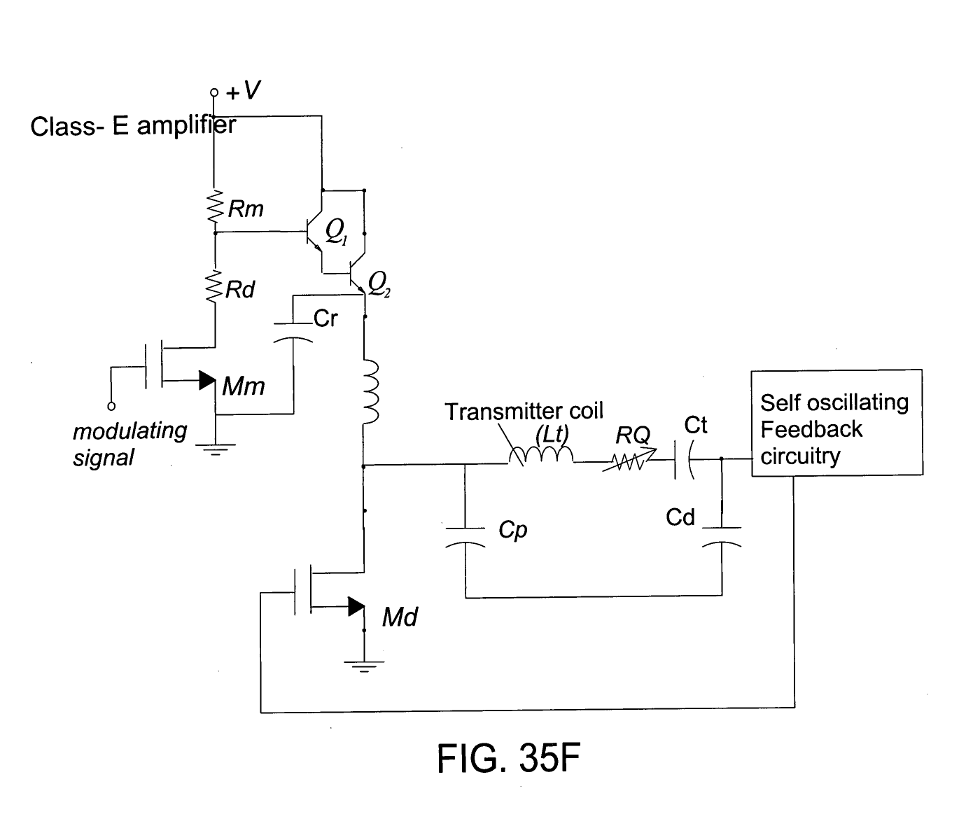

In one embodiment, the implanted stimulus-receiver 34C may be a system which is RF coupled combined with a power source. One such system is also disclosed in commonly owned patent application Ser. No. 10/195,961 which is incorporated herein in its entirety. In this embodiment, the implanted stimulus-receiver 34C contains high value, small sized capacitor(s) for storing charge and delivering electrical stimulation pulses for up to several hours by itself, once the capacitors are charged. The packaging is shown in conjunction with FIG. 35A. Using mostly hybrid components and appropriate packaging, the implanted portion of the system described below is conducive to miniaturization. As shown in FIG. 35A, a solenoid coil 382 wrapped around a ferrite core 380 is used as the secondary of an air-gap transformer for receiving power and data to the implanted device. The primary coil is external to the body. Since the coupling between the external transmitter coil and receiver coil 382 may be weak, a high-efficiency transmitter/amplifier is used in order to supply enough power to the receiver coil 382. Class-D or Class-E power amplifiers may be used for this purpose. The coil for the external transmitter (primary coil) may be placed in the pocket of a customized garment, as was previously shown.

One embodiment of the implanted lead stimulus-receiver and transmitter system are described in relation to FIG. 35B. As shown in FIG. 35B, the received signal after being picked by the resonant tank circuit comprising of inductor 256 and capacitor 271, goes through a rectifier 870. Even though a single diode 870 is shown in the figure, a diode bridge can be used for full-wave rectification, and the signal then goes through two series voltage regulators in order to generate the required supply voltages. The voltage regulators consist of rectifier, storage capacitor, and 4.5-V and 9-V shunt regulators implemented using Zenor diodes and resistors (not shown in FIG. 35B). Bipolar transistors and diodes with high breakdown voltages are used to provide protection from high input voltages. Clock 266 is regenerated from the radio-frequency (RF) carrier by taking the peak amplitude of sinusoidal carrier input and generating a 4.5 V square wave output. Data detection circuitry is comprised using a low-pass filter (LPF), a high-pass filter (HPF), and a Schmitt trigger for envelope detection and noise suppression. The low-pass filter is necessary in order to extract the envelope from the high frequency carrier. Finally, the output circuit contains charge-balance circuitry, stimulus current regulator circuitry, and startup circuitry.

As shown in FIG. 35B, a Class-D or Class E driver can be used in the external transmitter. A typical workable Class D driver is shown in FIG. 35C. Class-D transmitter can drive the loads efficiently, and can supply a constant driving source so that the link output voltage, or current, remains stable. A Class-D transmitter can drive these loads efficiently because it can supply a constant source which is independent of the load. It simply switches the input of the link between the two terminals of the power supply. Reactive loads and load variation due to changing coupling should not affect its output level.

Even though both Class-D and Class-E transmitters are highly efficient, the Class-E operation of one presently preffered embodiment is explained in relation to FIGS. 35D, 35E, and 35F. A basic circuit of Class-E amplifier is shown in FIG. 35D and its equivalent is shown in FIG. 35E. The “Class E” refers to a tuned power amplifier composed of a single-pole switch and a load network. The switch consists of a transistor or combination of transistors and diodes that are driven on and off at the carrier frequency of the signal to be amplified. In its most basic form, the load network consists of a resonant circuit in series with the load, and a capacitor which shunts the switch, FIGS. 35D and 35E. The total shunt capacitance is due to what is inherent in the transistor (C1) and added by the load network (C2). The collector or voltage waveform is then determined by the switch when it is on, and by the transient response of the load network when the switch is off.

In comparison, classes A, B, and C refer to amplifiers in which the transistors act as current sources; sinusoidal collector voltages are maintained by the parallel-tuned output circuit. If the transistors are driven hard enough to saturate, they cease to be current sources; however, the sinusoidal collector voltage remains. Class D is characterized by two (or more) pole switching configuration that define either a voltage current waveform without regard for the load network. Class D employs band-pass filtering. Table 3 below, compares the power and efficiency between different classes of amplifiers.

TABLE 3

| Class | Pₘₐₓ | Efficiency | Comments |
|---|---|---|---|
| A | 0.125 | 50% | 360° conduction angle |
| B | 0.125 | 78.5% | 180° conduction angle |
| C | 0.0981 | 89.6% | 120° conduction angle |
| D | 0.318 | 100% | uses two devices with 1 A |
| | | | peak current |
| E | 0.0981 | 100% | Optimum 50% duty cycle |

Class E power amplifiers (as well as Class D and saturating Class C power amplifiers) might more appropriately be called power converters. In these circuits, the driving signal causes switching of the transistor, but there is no relationship between the amplitudes of the driving signal and the output signal. In Class E amplifiers, there is no clear source of voltage or current, as in classes A, B, C, and D amplifiers. The collector voltage waveform is a function of the current charging the capacitor, and current is function of the voltage on the load, which is in turn a function of the collector voltage. All parameters are interrelated. A typical workable Class-E driver is shown in FIG. 35F.

As also shown schematically in FIG. 35B, the external pulse generator-transmitter can be operated via selecting a stored predetermined program 261 or by adjusting the stimulating program parameters individually 265. The range of electrical parameters that can be adjusted by the patient is defined by the attending physician, and parameters outside of that range are “locked out” to the patient via software. As also noted in FIG. 35B, there is an optional wireless communications module 255 for remotely communicating with the external pulse generator-transmitter.

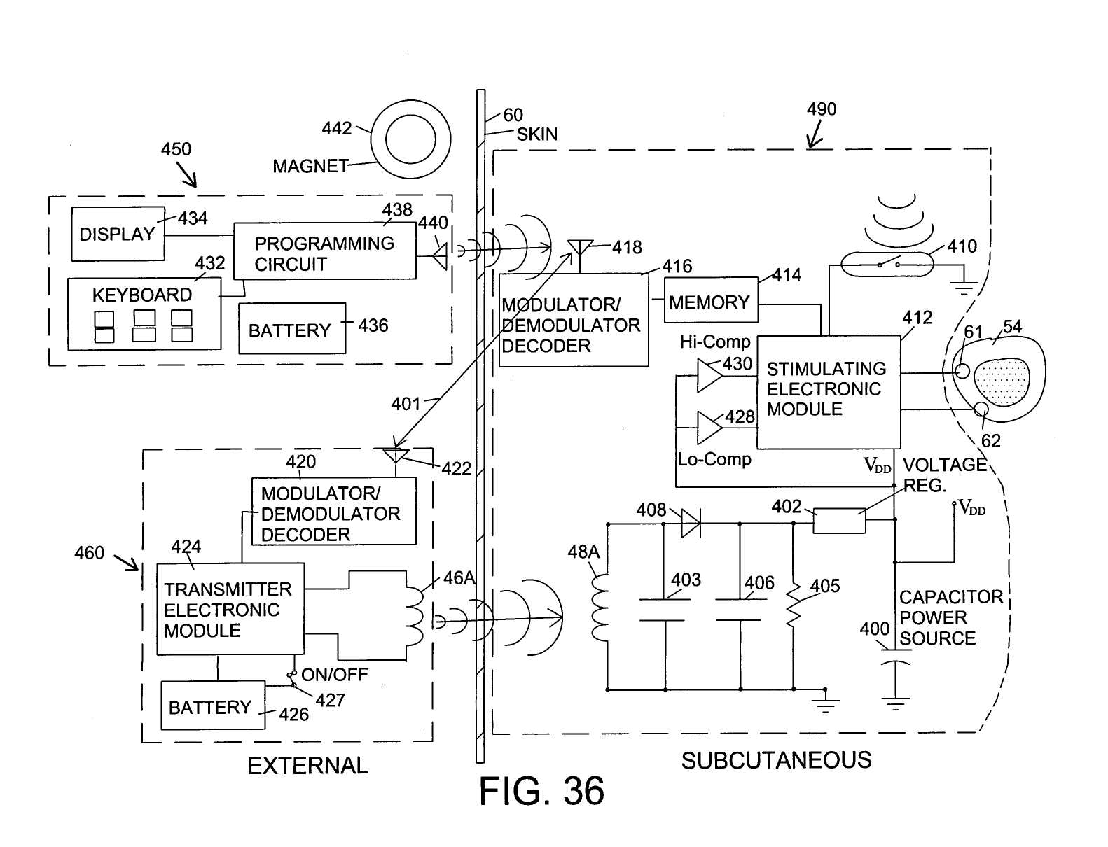

Another embodiment of implanted stimulus-receiver and the system is shown in conjunction with FIG. 36. In this embodiment of the implanted stimulus-receiver 490, the receiving inductor 48A and tuning capacitor 403 are tuned to the frequency of the transmitter. The diode 408 rectifies the AC signals, and a small sized capacitor 406 is utilized for smoothing the input voltage V_{I} fed into the voltage regulator 402. The output voltage V_{D} of regulator 402 is applied to capacitive energy power supply and source 400 which establishes source power V_{DD}. Capacitor 400 is a big value, small sized capacative energy source which is classified as low internal impedance, low power loss and high charge rate capacitor, such as Panasonic Model No. 641.

The refresh-recharge transmitter unit 460 includes a primary battery 426, an ON/Off switch 427, a transmitter electronic module 442, an RF inductor power coil 46A, a modulator/demodulator 420 and an antenna 422.

When the ON/OFF switch is on, the primary coil 46A is placed in close proximity to skin 60 and secondary coil 48A of the implanted stimulator 490. The inductor coil 46A emits RF waves establishing EMF wave fronts which are received by secondary inductor 48A. Further, transmitter electronic module 442 sends out command signals which are converted by modulator/demodulator decoder 420 and sent via antenna 422 to antenna 418 in the implanted stimulator 490. These received command signals are demodulated by decoder 416 and replied and responded to, based on a program in memory 414 (matched against a “command table” in the memory). Memory 414 then activates the proper controls and the inductor receiver coil 48A accepts the RF coupled power from inductor 46A.

The RF coupled power, which is alternating or AC in nature, is converted by the rectifier 408 into a high DC voltage. Small value capacitor 406 operates to filter and level this high DC voltage at a certain level. Voltage regulator 402 converts the high DC voltage to a lower precise DC voltage while capacitive power source 400 refreshes and replenishes.

When the voltage in capacative source 400 reaches a predetermined level (that is V_{DD} reaches a certain predetermined high level), the high threshold comparator 430 fires and stimulating electronic module 412 sends an appropriate command signal to modulator/decoder 416. Modulator/decoder 416 then sends an appropriate “fully charged” signal indicating that capacitive power source 400 is fully charged, is received by antenna 422 in the refresh-recharge transmitter unit 460.

In one mode of operation, the patient may start or stop stimulation by waving the magnet 442 once near the implant. The magnet emits a magnetic force Lₘ which pulls reed switch 410 closed. Upon closure of reed switch 410, stimulating electronic module 412 in conjunction with memory 414 begins the delivery (or cessation as the case may be) of controlled electronic stimulation pulses to the cortical neural tissue 54 via electrodes 61, 62, 63, 64. In another mode (AUTO), the stimulation is automatically delivered to the implanted lead based upon programmed ON/OFF times.

The programmer unit 450 includes keyboard 432, programming circuit 438, rechargeable battery 436, and display 434. The physician or medical technician programs programming unit 450 via keyboard 432. This program regarding the frequency, pulse width, modulation program, ON time etc. is stored in programming circuit 438. The programming unit 450 must be placed relatively close to the implanted stimulator 490 in order to transfer the commands and programming information from antenna 440 to antenna 418. Upon receipt of this programming data, modulator/demodulator and decoder 416 decodes and conditions these signals, and the digital programming information is captured by memory 414. This digital programming information is further processed by stimulating electronic module 412. In the DEMAND operating mode, after programming the implanted stimulator, the patient turns ON and OFF the implanted stimulator via hand held magnet 442 and the reed switch 410. In the automatic mode (AUTO), the implanted stimulator turns ON and OFF automatically according to the programmed values for the ON and OFF times.

Other simplified versions of such a system may also be used. For example, a system such as this, where a separate programmer is eliminated, and simplified programming is performed with a magnet and reed switch, can also be used.

### Programmer-Less Implantable Pulse Generator (IPG)

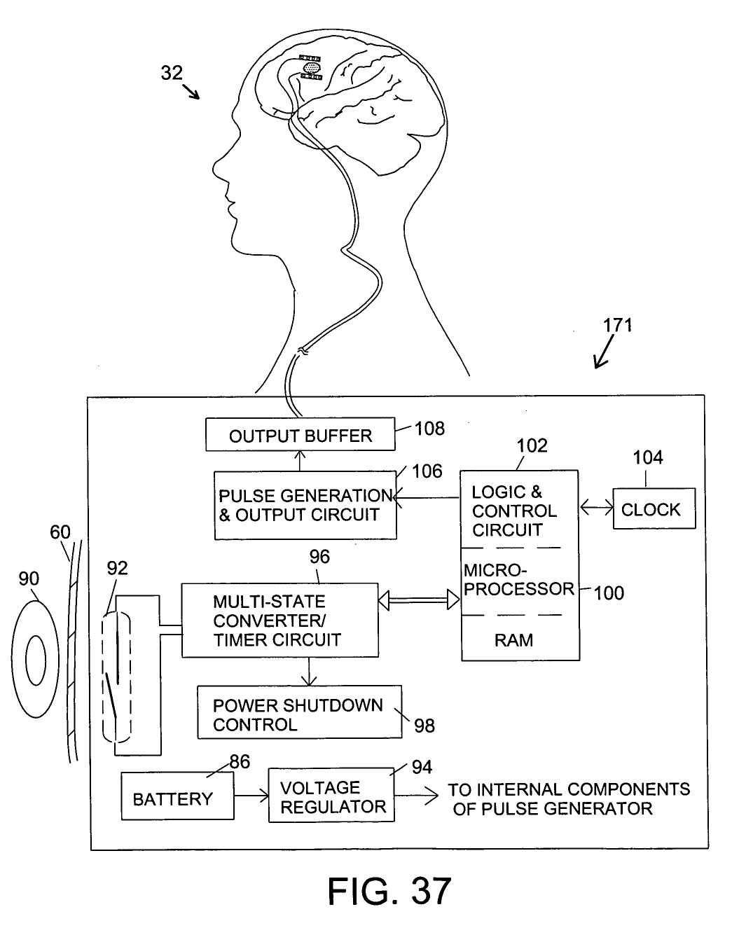

In one embodiment, a programmer-less implantable pulse generator (IPG) may be used, as disclosed in applicant's commonly assigned U.S. Pat. No. 6,760,626 B1, which is incorporated herein by reference. In this embodiment, shown in conjunction with FIG. 37, the implantable pulse generator 171 is provided with a reed switch 92 and memory circuitry 102. The reed switch 92 being remotely actuable by means of a magnet 90 brought into proximity of the pulse generator 171, in accordance with common practice in the art. In this embodiment, the reed switch 92 is coupled to a multi-state converter/timer circuit 96, such that a single short closure of the reed switch can be used as a means for non-invasive encoding and programming of the pulse generator 171 parameters.

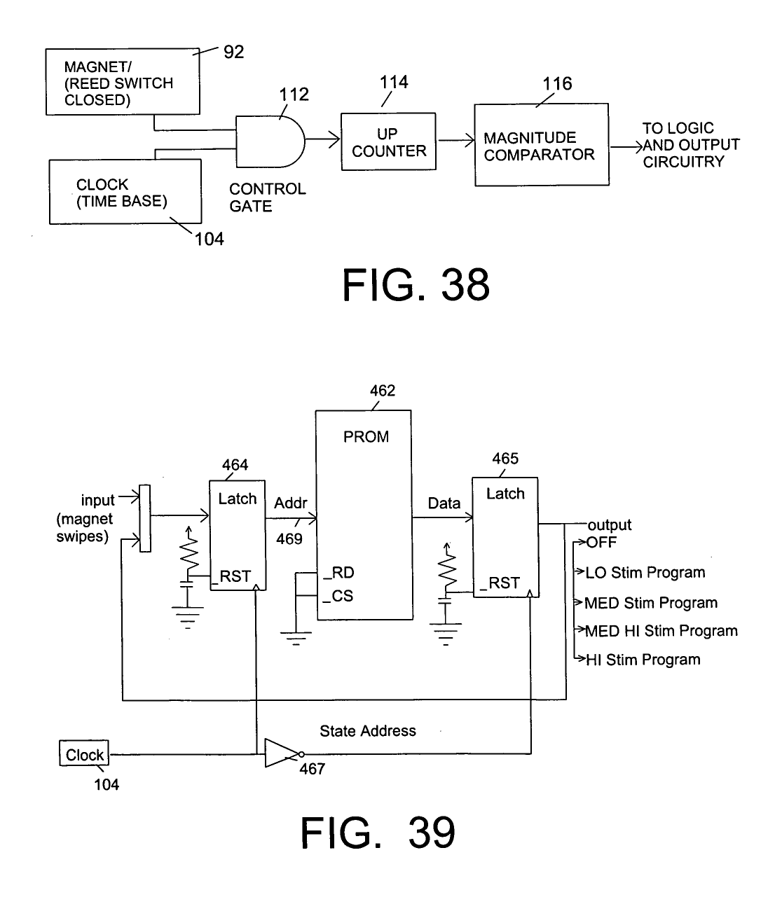

In one embodiment, shown in conjunction with FIG. 38, the closing of the reed switch 92 triggers a counter. The magnet 90 and timer are ANDed together. The system is configured such that during the time that the magnet 82 is held over the pulse generator 171, the output level goes from LOW stimulation state to the next higher stimulation state every 5 seconds. Once the magnet 82 is removed, regardless of the state of stimulation, an application of the magnet, without holding it over the pulse generator 171, triggers the OFF state, which also resets the counter.

Once the prepackaged/predetermined logic state is activated by the logic and control circuit 102, as shown in FIG. 37, the pulse generation and amplification circuit 106 deliver the appropriate electrical pulses to the cortical neural tissues 54 of the patient via an output buffer 108. In this embodiment, the electrode configuration for delivery of output pulses is preset Timing signals for the logic and control circuit 102 of the pulse generator 171 are provided by a crystal oscillator 104. The battery 86 of the pulse generator 171 has terminals connected to the input of a voltage regulator 94. The regulator 94 smoothes the battery output and supplies power to the internal components of the pulse generator 171. A microprocessor 100 controls the program parameters of the device, such as the voltage, pulse width, frequency of pulses, on-time and off-time. The microprocessor may be a commercially available, general purpose microprocessor or microcontroller, or may be a custom integrated circuit device augmented by standard RAM/ROM components.

In one embodiment, there are four stimulation states. A larger (or lower) number of states can be achieved using the same methodology, and such is considered within the scope of the invention. These four states are, LOW stimulation state, LOW-MED stimulation state, MED stimulation state, and HIGH stimulation state. Examples of stimulation parameters (delivered to the cortical tissues) for each state are as follows,

### For Enhancing Neuroplasticity (Post-Stroke Patients)

LOW stimulation state example is,

| | |
|---|---|
| Current output: | 0.30 milli-Amps. |
| Pulse width: | 50 micro-secs. |
| Pulse frequency: | 50 Hertz |
| Cycles: | 5 sec. on-time and 5 sec. off-time in repeating cycles. |

LOW-MED stimulation state example is,

| | |
|---|---|
| Current output: | 0.50 milli-Amps. |
| Pulse width: | 70 micro-secs. |
| Pulse frequency: | 75 Hertz |
| Cycles: | 10 sec. on-time and 5 sec. off-time in repeating cycles. |

MED stimulation state example is,

| | |
|---|---|
| Current output: | 0.75 milli-Amps. |
| Pulse width: | 80 micro-secs. |
| Pulse frequency: | 90 Hertz |
| Cycles: | ON continuously |

HIGH stimulation state example is,

| | |
|---|---|
| Current output: | 1.0 milli-Amp. |
| Pulse width: | 100 micro-secs. |
| Pulse frequency: | 120 Hertz |
| Cycles: | 20 sec. on-time and 5 sec. off-time in repeating cycles. |

These prepackaged/predetermined programs are mearly examples, and the actual stimulation parameters will deviate from these depending on the treatment application.

### For Parkinson's Disease

LOW stimulation state example is,

| | |
|---|---|
| Current output: | 1.5 milliAmps. |
| Pulse width: | 125 micro-secs. |
| Pulse frequency: | 20 Hz |
| Cycles: | 20 min. on-time and 5 min. off-time in repeating |
| | cycles. |

LOW-MED stimulation state example is,

| | |
|---|---|
| Current output: | 2.5 milli-Amps. |
| Pulse width: | 200 micro-secs. |
| Pulse frequency: | 50 Hz |
| Cycles: | ON continuously for 2.5 hours |

MED stimulation state example is,

| | |
|---|---|
| Current output: | 3.5 milli-Amps. |
| Pulse width: | 300 micro-secs. |
| Pulse frequency: | 75 Hz |
| Cycles: | ON continuously for 4 hours |

HIGH stimulation state example is,

| | |
|---|---|
| Current output: | 5.0 milli-Amps. |
| Pulse width: | 400 micro-secs. |
| Pulse frequency: | 100 Hz |
| Cycles: | ON continuously for 6 hours |

These prepackaged/predetermined programs are mearly examples, and the actual stimulation parameters will deviate from these depending on the treatment application.

It will be readily apparent to one skilled in the art, that other schemes can be used for the same purpose. For example, instead of placing the magnet 90 on the pulse generator 171 for a prolonged period of time, different stimulation states can be encoded by the sequence of magnet applications. Accordingly, in an alternative embodiment there can be three logic states, OFF, LOW stimulation (LS) state, and HIGH stimulation (HS) state. Each logic state again corresponds to a prepackaged/predetermined program such as presented above. In such an embodiment, the system could be configured such that one application of the magnet triggers the generator into LS State. If the generator is already in the LS state then one application triggers the device into OFF State. Two successive magnet applications triggers the generator into MED stimulation state, and three successive magnet applications triggers the pulse generator in the HIGH Stimulation State. Subsequently, one application of the magnet while the device is in any stimulation state, triggers the device OFF.

FIG. 39 shows a representative digital circuitry used for the basic state machine circuit. The circuit consists of a PROM 462 that has part of its data fed back as a state address. Other address lines 469 are used as circuit inputs, and the state machine changes its state address on the basis of these inputs. The clock 104 is used to pass the new address to the PROM 462 and then pass the output from the PROM 462 to the outputs and input state circuits. The two latches 464, 465 are operated 180° out of phase to prevent glitches from unexpectedly affecting any output circuits when the ROM changes state. Each state responds differently according to the inputs it receives.

The advantage of this embodiment is that it is cheaper to manufacture than a fully programmable implantable pulse generator (IPG).

### Microstimulator

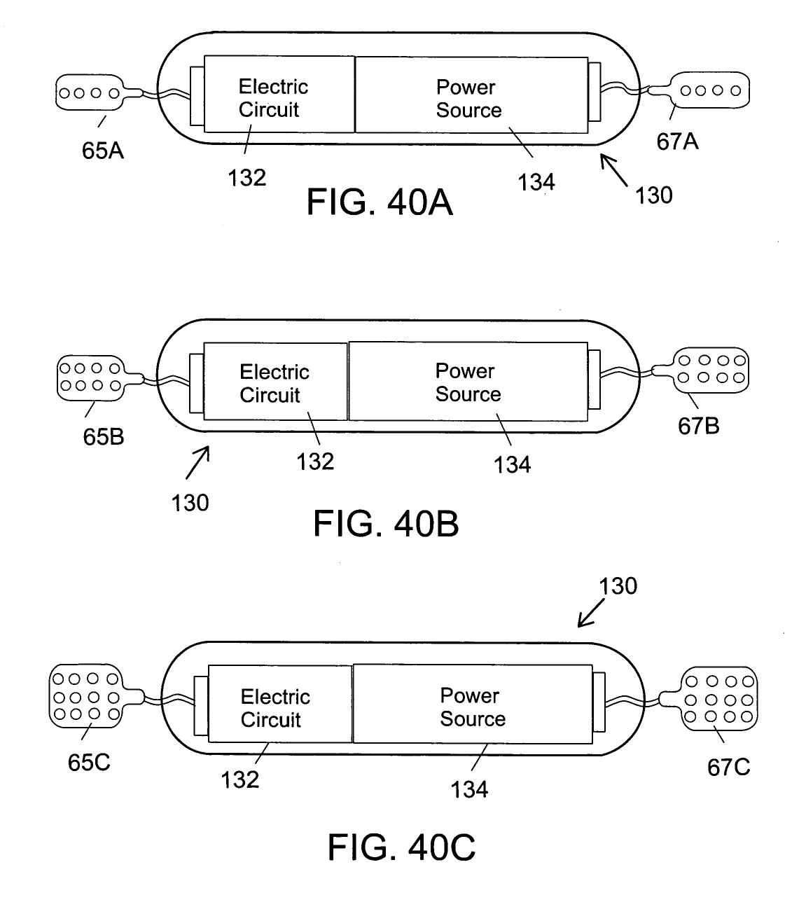

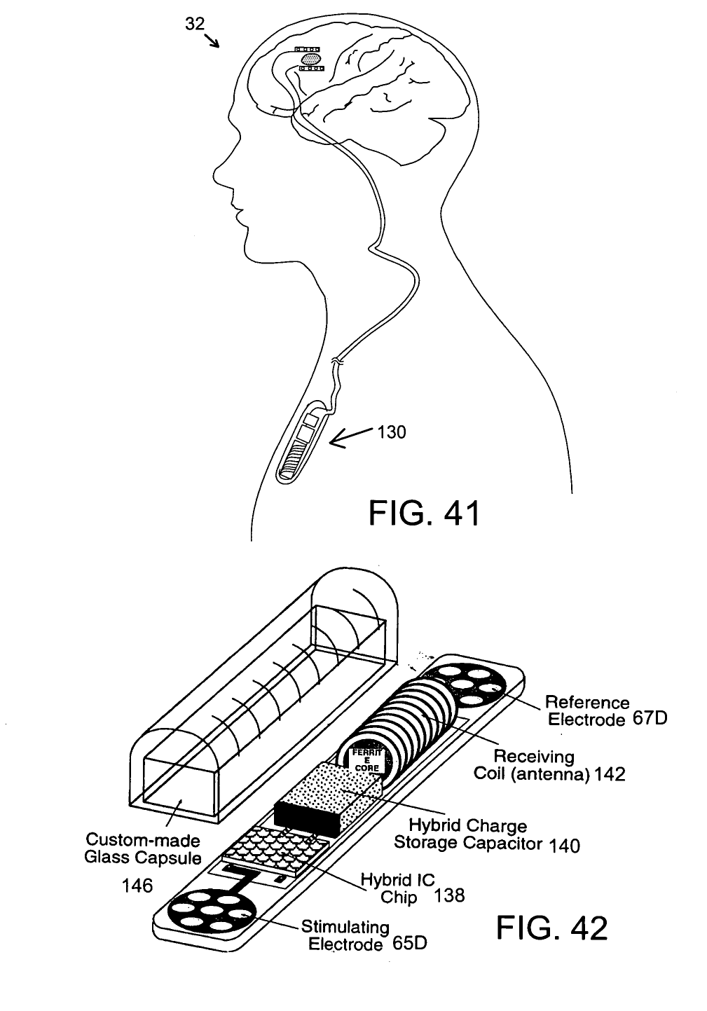

In one embodiment, a microstimulator 130 may be used for providing pulses to the cortical tissues 54. Shown in conjunction with FIG. 40A, is a microstimulator where the electrical circuitry 132 and power source 134 are encased in a miniature hermetically sealed enclosure, and only the electrodes 65A, 67A are exposed. FIGS. 40B and 40C depict the same microstimulator, except that the electrode array and circuitry is configured to have a larger number of electrodes. The embodiment shown in FIG. 40B comprises eight pairs of electrodes, and the embodiment shown in FIG. 40C comprises 12 pairs of electrodes. Because of its small size, the whole microstimulator may be implanted closer in proximity to tissues being stimulated such as cortical tissues. In one example, the microstimulator may be implanted approximately behind the ear, as was shown in conjunction with FIGS. 27A, 27B, and 27C. Alternatively as shown in conjunction with FIG. 41, the microstimulator may be implanted at a different site, and connected to the electrodes via conductors insulated with silicone and polyurethane.

Shown in reference with FIG. 42 is the overall structure of an implantable microstimulator 130. It consists of a micromachined silicon substrate that incorporates two stimulating electrodes which are the cathode and anode of a bipolar stimulating electrode pair 65D, 67D; a hybrid-connected tantalum chip capacitor 140 for power storage; a receiving coil 142; a bipolar-CMOS integrated circuit chip 138 for power regulation and control of the microstimulator; and a custom made glass capsule 146 that is electrostatically bonded to the silicon carrier to provide a hermetic package for the receiver-stimulator circuitry and hybrid elements. The stimulating electrode(s) 65D, 67D resides outside of the package and feedthroughs are used to connect the internal electronics to the electrodes.

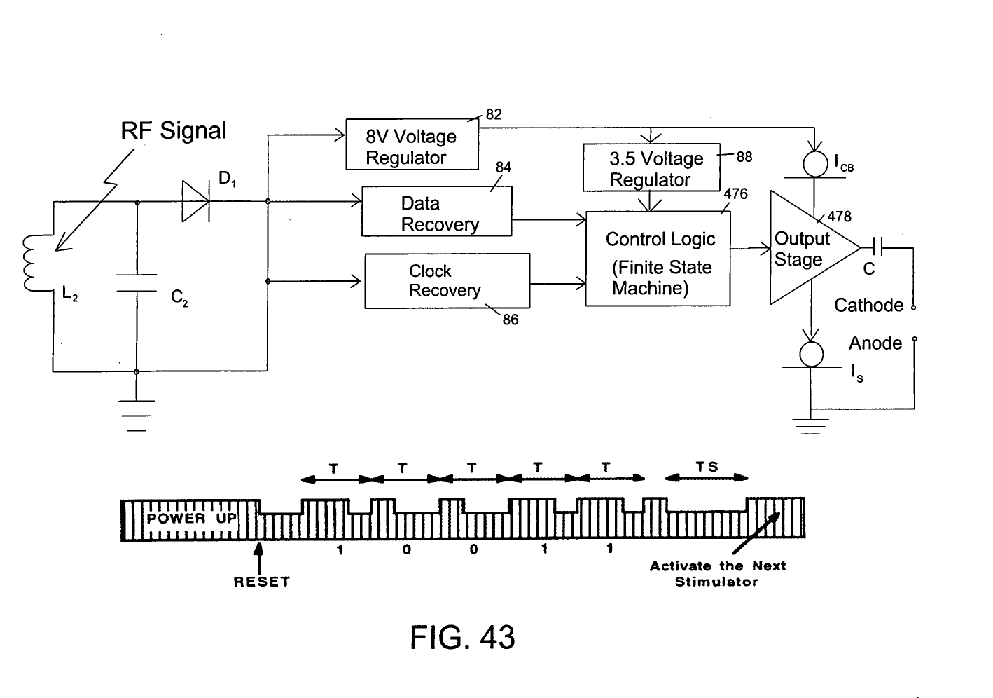

FIG. 43 shows the overall system electronics required for the microstimulator, and the power and data transmission protocol used for radiofrequency telemetry. The circuit receives an amplitude modulated RF carrier from an external transmitter and generates 8-V and 4-V dc supplies, generates a clock from the carrier signal, decodes the modulated control data, interprets the control data, and generates a constant current output pulse when appropriate. The RF carrier used for the telemetry link has a nominal frequency of around 1.8 MHz, and is amplitude modulated to encode control data. Logical “1” and “0” are encoded by varying the width of the amplitude modulated carrier, as shown in the bottom portion of FIG. 43. The carrier signal is initially high when the transmitter is turned on and sets up an electromagnetic field inside the transmitter coil. The energy in the field is picked up by receiver coils 142, and is used to charge the hybrid capacitor 140. The carrier signal is turned high and then back down again, and is maintained at the low level for a period between 1-200 μsec. The microstimulator 130 will then deliver a constant current pulse into the cortical neural tissue through the stimulating electrode(s) 65D, 67D for the period that the carrier is low. Finally, the carrier is turned back high again, which will indicate the end of the stimulation period to the microstimulator 130, thus allowing it to charge its capacitor 140 back up to the on-chip voltage supply.

On-chip circuitry has been designed to generate two regulated power supply voltages (4V and 8V) from the RF carrier, to demodulate the RF carrier in order to recover the control data that is used to program the microstimulator, to generate the clock used by the on-chip control circuitry, to deliver a constant current through a controlled current driver into the nerve tissue, and to control the operation of the overall circuitry using a low-power CMOS logic controller.

### Programmable Implantable Pulse Generator (IPG)

In one embodiment, a fully programmable implantable pulse generator (IPG), capable of generating stimulation and blocking pulses may be used. One such system is also disclosed in commonly owned patent application Ser. No. 10/841,995 which is incorporated herein in its entirety. Shown in conjunction with FIGS. 44A, 44B, and 44C, the implantable pulse generator unit 391 is preferably a microprocessor based device, where the entire circuitry is encased in a hermetically sealed titanium can. The circuitry in FIGS. 44A, 44B, and 44C is similar, except FIG. 44B depicts more than one channel of output which shown as output circuitry 385A and 385B. The block diagram depicted in FIG. 44C also comprises sensing circuitry, with multiple channels of output circuitry and sensing circuitry. As shown in the overall block diagram (FIG. 44A), the logic & control unit 398 provides the proper timing for the output circuitry 385 to generate electrical pulses that are delivered to electrodes 61A, 62A, 61B and 62B via a lead 40. Programming of the implantable pulse generator (IPG) is done via an external programmer 85, as described later. Once activated or programmed via an external programmer 85, the implanted pulse generator 391 provides appropriate electrical stimulation pulses to the cortical neural tissues 54 via electrodes 61A, 62A, 61B and 62B. This embodiment may also be adapted and configured for lead(s) comprising multiple electrodes.

This embodiment also comprises predetermined/pre-packaged programs. Examples of four stimulation states were given in the previous section, under “Programmer-less Implantable Pulse Generator (IPG)”. These predetermined/pre-packaged programs comprise unique combinations of pulse amplitude, pulse width, pulse morphology, pulse frequency, ON-time and OFF-time, and electrode configurations for stimulations. Any number of predetermined/pre-packaged programs, even 100, can be stored in the implantable pulse generator of this invention, and are considered within the scope of the invention.

Examples of additional predetermined/pre-packaged programs are:
For enhancing neuroplasticity (post-stroke patients)

Program one:

| | |
|---|---|
| Current output: | 0.25 milli-Amps. |
| Pulse width: | 40 micro-secs. |
| Pulse frequency: | 50 Hertz |
| Cycles: | 10 sec. on-time and 5 sec. off-time in repeating cycles. |

Program two:

| | |
|---|---|
| Current output: | 0.40 milli-Amps. |
| Pulse width: | 50 micro-secs. |
| Pulse frequency: | 60 Hertz |
| Cycles: | 12 sec. on-time and 4 sec. off-time in repeating cycles. |

Program three:

| | |
|---|---|
| Current output: | 0.50 milli-Amps. |
| Pulse width: | 70 micro-secs. |
| Pulse frequency: | 75 Hertz |
| Cycles: | 15 sec. on-time and 5 sec. off-time in repeating cycles. |

Program four:

| | |
|---|---|
| Current output: | 0.60 milli-Amps. |
| Pulse width: | 85 micro-secs. |
| Pulse frequency: | 90 Hertz |
| Cycles: | ON continuously |

Program five:

| | |
|---|---|
| Current output: | 0.75 milli-Amps. |
| Pulse width: | 100 micro-secs. |
| Pulse frequency: | 125 Hertz |
| Cycles: | 25 sec. on-time and 5 sec. off-time in repeating cycles. |

Program six (complex pulses):

| | |
|---|---|
| Current output: | 0.50 milli-Amps. |
| Pulse width: | 150 micro-secs. |
| Pulse frequency: | 50 Hertz |
| Pulse type: | step pulses |
| Cycles: | 20 sec. on-time and 5 sec. off-time in repeating cycles. |

Program seven (complex pulses):

| | |
|---|---|
| Current output: | 0.75 milli-Amps. |
| Pulse width: | 150 micro-secs. |
| Pulse frequency: | 80 Hertz |
| Pulse type: | step pulses |
| Cycles: | 25 sec. on-time and 5 sec. off-time in repeating cycles. |

Program eight (complex pulse train):

| | |
|---|---|
| Current output: | 0.85 milli-Amps. |
| Pulse width: | 200 micro-secs. |
| Pulse frequency: | 125 Hertz |
| Pulse type: | step pulses with alternating pulse train |
| | (as shown in FIG. 52H) |
| Cycles: | ON continuously |

These pre-packaged/predetermined programs are mearly examples, and the actual stimulation parameters will deviate from these depending on the treatment application and physician preference.
For Parkinson's disease

Program one:

| | |
|---|---|
| Current output: | 0.75 milli-Amps. |
| Pulse width: | 50 micro-seconds |
| Pulse frequency: | 25 Hertz |
| Cycles: | 5 min. on-time and 30 sec. off-time in |
| | repeating cycles for 12 hours. |

Program two:

| | |
|---|---|
| Current output: | 1.0 milli-Amps, |
| Pulse width: | 150 micro-seconds |
| Pulse frequency: | 40 Hertz |
| Cycles: | ON continuously for 2.5 hours |

Program three:

| | |
|---|---|
| Current output: | 2.5 milli-Amps. |
| Pulse width: | 200 micro-seconds |
| Pulse frequency: | 75 Hertz |
| Cycles: | ON continuously for 4.0 hours |

Program four:

| | |
|---|---|
| Current output: | 3.5 milli-Amps, |
| Pulse width: | 500 micro-seconds |
| Pulse frequency: | 85 Hertz |
| Cycles: | ON continuously for 6.0 hours |

Program five:

| | |
|---|---|
| Current output: | 5.0 milli-Amps, |
| Pulse width: | 0.50 milli-second |
| Pulse frequency: | 100 Hertz |
| Cycles: | ON continuously for 8.5 hours |

Program six (complex pulses):

| | |
|---|---|
| Current output: | 2.5 milli-Amps. |
| Pulse width: | 300 micro-seconds |
| Pulse frequency: | 50 Hertz |
| Pulse type: | step pulses |
| Cycles: | ON continuously for 4.0 hours |

Program seven (complex pulses):

| | |
|---|---|
| Current output: | 3.5 milli-Amps. |
| Pulse width: | 450 micro-seconds |
| Pulse frequency: | 75 Hertz |
| Pulse type: | step pulses |
| Cycles: | ON continuously for 6.0 hours |

Program eight (complex pulse train):

| | |
|---|---|
| Current output: | 4.0 milli-Amps. |
| Pulse width: | 0.50 milli-sec. |
| Pulse frequency: | 85 Hertz |
| Pulse type: | step pulses with alternating pulse train |
| | (as shown in FIG. 52H) |
| Cycles: | ON continuously for 8.0 hours |

These pre-packaged/predetermined programs are mearly examples, and the actual stimulation parameters will deviate from these depending on the treatment application and physician preference. One advantage of predetermined/pre-packaged program is that it can be readily activated by a program number. A simple version of a programmer, adapted to activate only a limited number of predetermined/pre-packaged programs may also be supplied to the patient.

In addition, each parameter may be individually adjusted and stored in the memory 394. The range of programmable electrical stimulation parameters include both stimulating and blocking frequencies, are shown in table four below and are considered within the scope of this application.

TABLE 4
Programmable electrical parameter range

| PARAMETER | RANGE |
|---|---|
| Pulse Amplitude | 0.1 Volt-25 Volts |
| Pulse width | 20 μS-5 mSec. |
| Stim. Frequency | 5 Hz-150 Hz |
| Freq. for blocking | 100 to 1,000 Hz |
| On-time | 5 Secs-24 hours |
| Off-time | 5 Secs-24 hours |

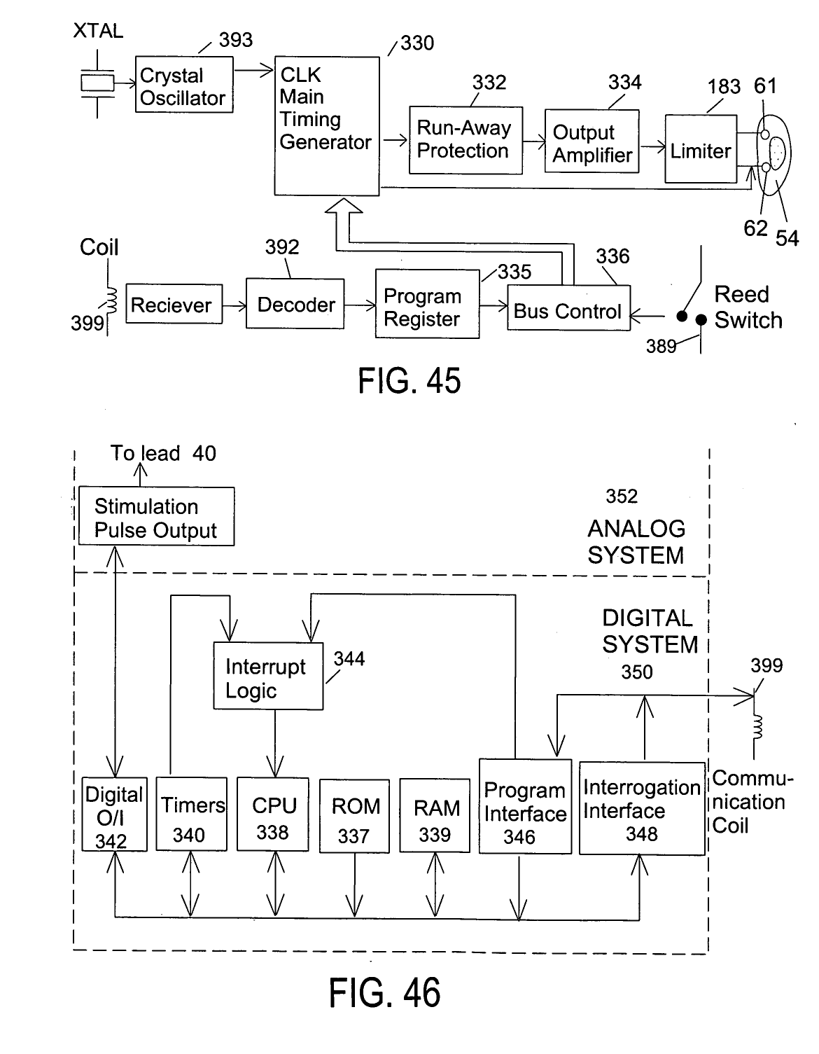

Shown in conjunction with FIGS. 45 and 46, the electronic stimulation module comprises both digital 350 and analog 352 circuits. A main timing generator 330 (shown in FIG. 45), controls the timing of the analog output circuitry for delivering neuromodulating pulses to the cortical neural tissues 54, via output amplifier 334. Limiter 183 prevents excessive stimulation energy from getting into the cortical neural tissues 54. The main timing generator 330 receiving clock pulses from crystal oscillator 393. Main timing generator 330 also receiving input from programmer 85 via coil 399. FIG. 36 highlights other portions of the digital system such as CPU 338, ROM 337, RAM 339, program interface 346, interrogation interface 348, timers 340, and digital O/I 342.

Most of the digital functional circuitry 350 is on a single chip (IC). This monolithic chip along with other IC's and components such as capacitors and the input protection diodes are assembled together on a hybrid circuit. As well known in the art, hybrid technology is used to establish the connections between the circuit and the other passive components. The integrated circuit is hermetically encapsulated in a chip carrier. A coil 399 situated under the hybrid substrate is used for bidirectional telemetry. The hybrid and battery 397 are encased in a titanium can 65. This housing is a two-part titanium capsule that is hermetically sealed by laser welding. Alternatively, electron-beam welding can also be used. The header 79 is a cast epoxy-resin with hermetically sealed feed-through, and form the lead 40 connection block.

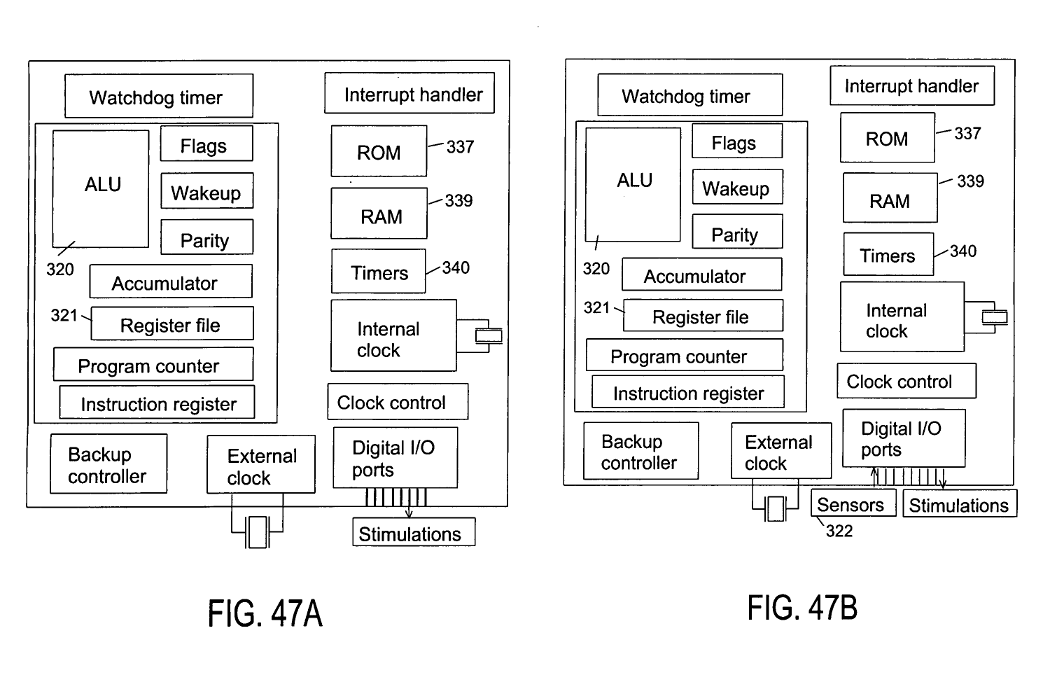

For further details, FIG. 47A highlights the general components of an 8-bit microprocessor as an example. It will be obvious to one skilled in the art that higher level microprocessor, such as a 16-bit or 32-bit may be utilized, and is considered within the scope of this invention. It comprises a ROM 337 to store the instructions of the program to be executed and various programmable parameters, a RAM 339 to store the various intermediate parameters, timers 340 to track the elapsed intervals, a register file 321 to hold intermediate values, an ALU 320 to perform the arithmetic calculation, and other auxiliary units that enhance the performance of a microprocessor-based IPG system.

The size of ROM 337 and RAM 339 units are selected based on the requirements of the algorithms and the parameters to be stored. The number of registers in the register file 321 are decided based upon the complexity of computation and the required number of intermediate values. Timers 340 of different precision are used to measure the elapsed intervals. Even though this embodiment does not have external sensors to control timing, future embodiments may have sensors 322 to effect the timing as shown in conjunction with FIG. 47B.

In this embodiment, the two main components of microprocessor are the datapath and control. The datapath performs the arithmetic operation and the control directs the datapath, memory, and I/O devices to execute the instruction of the program. The hardware components of the microprocessor are designed to execute a set of simple instructions. In general the complexity of the instruction set determines the complexity of datapth elements and controls of the microprocessor.

In this embodiment, the microprocessor is provided with a fixed operating routine. Future embodiments may be provided with the capability of actually introducing program changes in the implanted pulse generator. The instruction set of the microprocessor, the size of the register files, RAM and ROM are selected based on the performance needed and the type of the algorithms used. In this application of pulse generator, in which several algorithms can be loaded and modified, Reduced Instruction Set Computer (RISC) architecture is useful. RISC architecture offers advantages because it can be optimized to reduce the instruction cycle which in turn reduces the run time of the program and hence the current drain. The simple instruction set architecture of RISC and its simple hardware can be used to implement any algorithm without much difficulty. Since size is also a major consideration, an 8-bit microprocessor is used for the purpose. As most of the arithmetic calculation are based on a few parameters and are rather simple, an accumulator architecture is used to save bits from specifying registers. Each instruction is executed in multiple clock cycles, and the clock cycles are broadly classified into five stages: an instruction fetch, instruction decode, execution, memory reference, and write back stages. Depending on the type of the instruction, all or some of these stages are executed for proper completion.

Initially, an optimal instruction set architecture is selected based on the algorithm to be implemented and also taking into consideration the special needs of a microprocessor based implanted pulse generator (IPG). The instructions are broadly classified into Load/store instructions, Arithmetic and logic instructions (ALU), control instructions and special purpose instructions.

The instruction format is decided based upon the total number of instructions in the instruction set. The instructions fetched from memory are 8 bits long in this example. Each instruction has an opcode field (2 bits), a register specifier field (3-bits), and a 3-bit immediate field. The opcode field indicates the type of the instruction that was fetched. The register specifier indicates the address of the register in the register file on which the operations are performed. The immediate field is shifted and sign extended to obtain the address of the memory location in load/store instruction. Similarly, in branch and jump instruction, the offset field is used to calculate the address of the memory location the control needs to be transferred to.

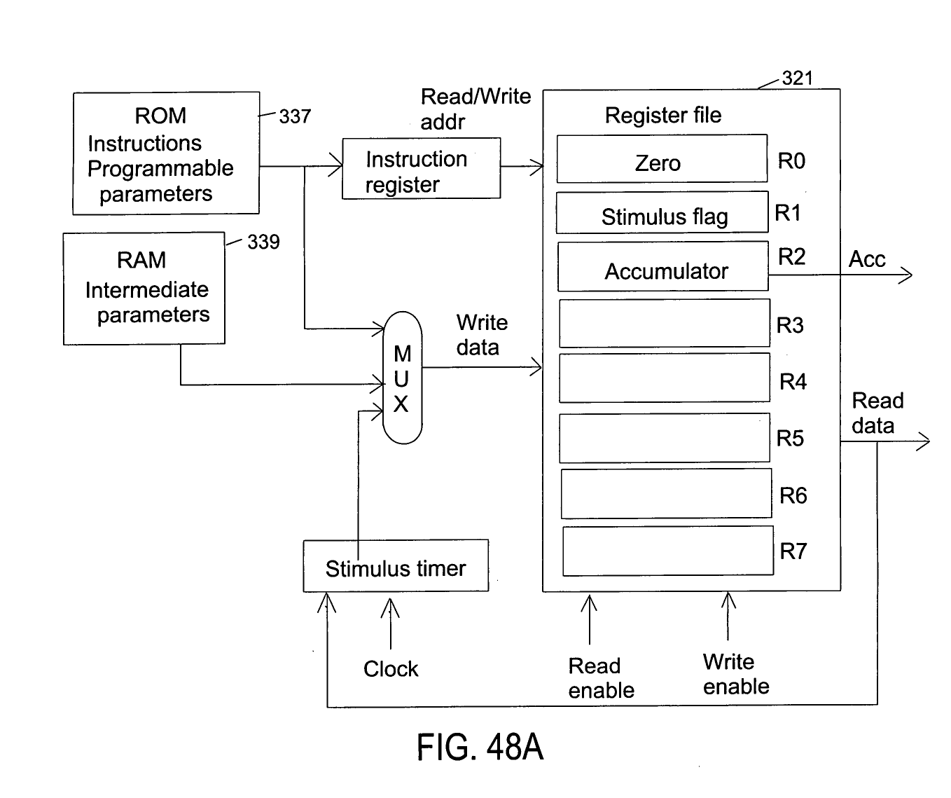

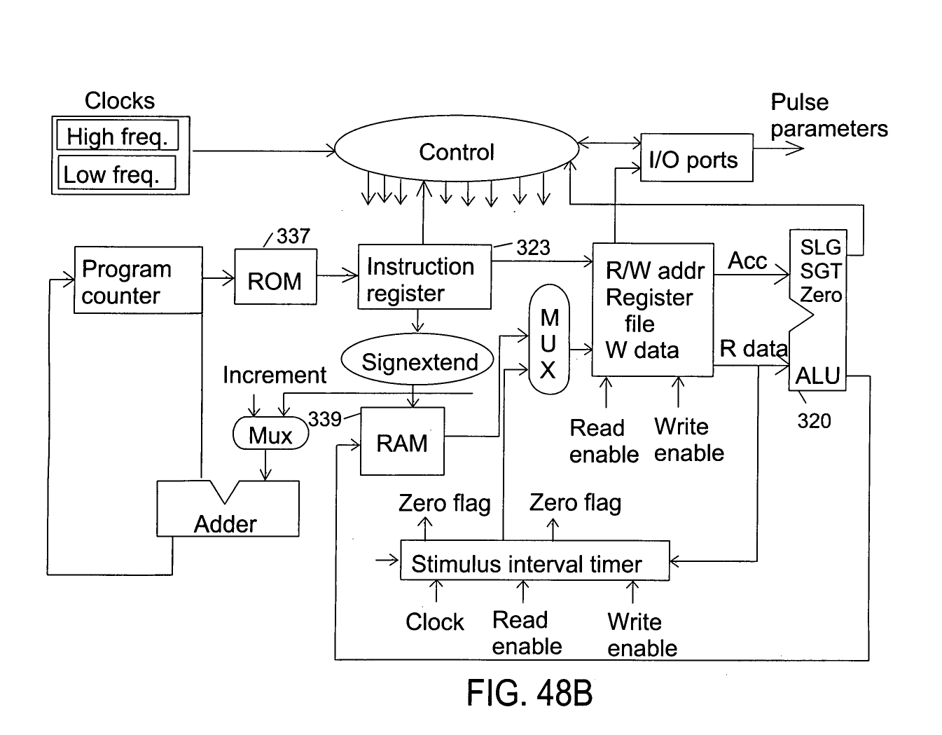

Shown in conjunction with FIG. 48A, the register file 321, which is a collection of registers in which any register can be read from or written to specifying the number of the register in the file. Based on the requirements of the design, the size of the register file is decided. For the purposes of implementation of stimulation pulses algorithms, a register file of eight registers is sufficient, with three special purpose register (0-2) and five general purpose registers (3-7), as shown in FIG. 48A. Register “0” always holds the value “zero”. Register “1” is dedicated to the pulse flags. Register “2” is an accumulator in which all the arithmetic calculations are performed. The read/write address port provides a 3-bit address to identify the register being read or written into. The write data port provides 8-bit data to be written into the registers either from ROM/RAM or timers. Read enable control, when asserted enables the register file to provide data at the read data port. Write enable control enables writing of data being provided at the write data port into a register specified by the read/write address.

Generally, two or more timers are required to implement the algorithm for the IPG. The timers are read and written into just as any other memory location. The timers are provided with read and write enable controls.

The arithmetic logic unit is an important component of the microprocessor. It performs the arithmetic operation such as addition, subtraction and logical operations such as AND and OR. The instruction format of ALU instructions consists of an opcode field (2 bits), a function field (2 bits) to indicate the function that needs to be performed, and a register specifier (3 bits) or an immediate field (4 bits) to provide an operand.

The hardware components discussed above constitute the important components of a datapath. Shown in conjunction with FIG. 48B, there are some special purpose registers such a program counter (PC) to hold the address of the instruction being fetched from ROM 337 and instruction register (IR) 323, to hold the instruction that is fetched for further decoding and execution. The program counter is incremented in each instruction fetch stage to fetch sequential instruction from memory. In the case of a branch or jump instruction, the PC multiplexer allows to choose from the incremented PC value or the branch or jump address calculated. The opcode of the instruction fetched (IR) is provided to the control unit to generate the appropriate sequence of control signals, enabling data flow through the datapath. The register specification field of the instruction is given as read/write address to the register file, which provides data from the specified field on the read data port. One port of the ALU is always provided with the contents of the accumulator and the other with the read data port. This design is therefore referred to as accumulator-based architecture. The sign-extended offset is used for address calculation in branch and jump instructions. The timers are used to measure the elapsed interval and are enabled to count down on a low-frequency clock. The timers are read and written into, just as any other memory location (FIG. 48B).

In a multicycle implementation, each stage of instruction execution takes one clock cycle. Since the datapath takes multiple clock cycles per instruction, the control must specify the signals to be asserted in each stage and also the next step in the sequence. This can be easily implemented as a finite state machine.

A finite state machine consists of a set of states and directions on how to change states. The directions are defined by a next-state function, which maps the current state and the inputs to a new state. Each stage also indicates the control signals that need to be asserted. Every state in the finite state machine takes one clock cycle. Since the instruction fetch and decode stages are common to all the instruction, the initial two states are common to all the instruction. After the execution of the last step, the finite state machine returns to the fetch state.

A finite state machine can be implemented with a register that holds the current stage and a block of combinational logic such as a PLA. It determines the datapath signals that need to be asserted as well as the next state. A PLA is described as an array of AND gates followed by an array of OR gates. Since any function can be computed in two levels of logic, the two-level logic of PLA is used for generating control signals.

The occurrence of a wakeup event initiates a stored operating routine corresponding to the event. In the time interval between a completed operating routine and a next wake up event, the internal logic components of the processor are deactivated and no energy is being expended in performing an operating routine.

A further reduction in the average operating current is obtained by providing a plurality of counting rates to minimize the number of state changes during counting cycles. Thus intervals which do not require great precision, may be timed using relatively low counting rates, and intervals requiring relatively high precision, such as stimulating pulse width, may be timed using relatively high counting rates.

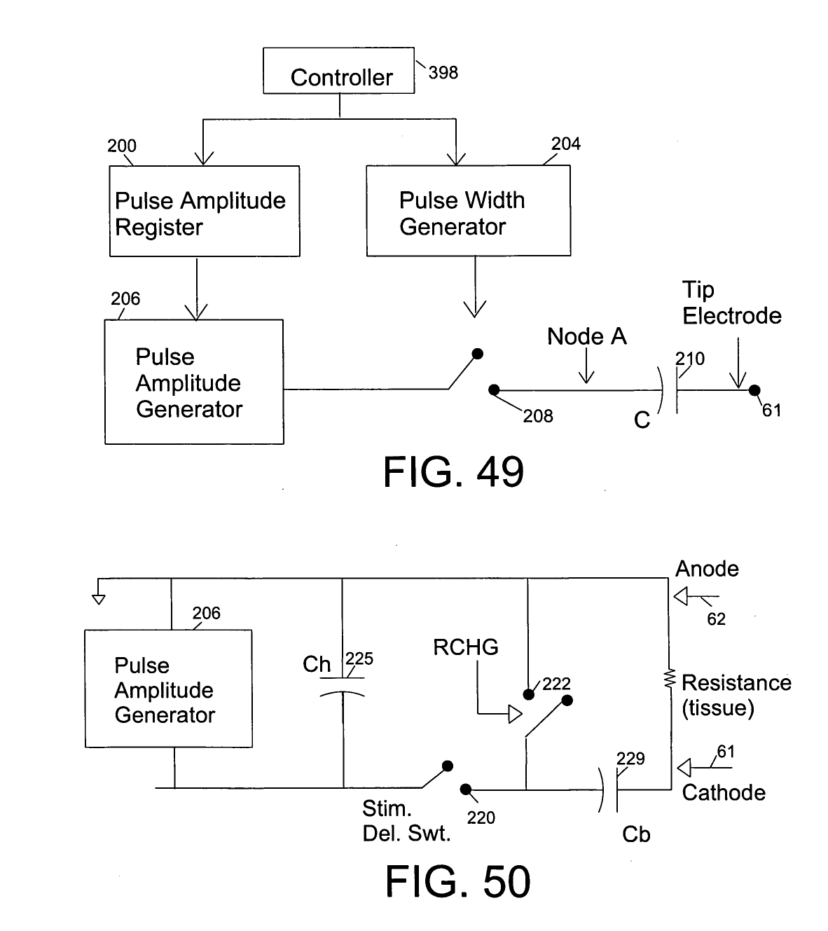

The logic and control unit 398 of the IPG controls the output amplifiers. The pulses have predetermined energy (pulse amplitude and pulse width) and are delivered at a time determined by the therapy stimulus controller. The circuitry in the output amplifier, shown in conjunction with (FIG. 49) generates an analog voltage or current that represents the pulse amplitude. The stimulation controller module initiates a stimulus pulse by closing a switch 208 that transmits the analog voltage or current pulse to the nerve tissue through the tip electrode 61 of the lead 40. The output circuit receiving instructions from the stimulus therapy controller 398 that regulates the timing of stimulus pulses and the amplitude and duration (pulse width) of the stimulus. The pulse amplitude generator 206 determines the configuration of charging and output capacitors necessary to generate the programmed stimulus amplitude. The output switch 208 is closed for a period of time that is controlled by the pulse width generator 204. When the output switch 208 is closed, a stimulus is delivered to the tip electrode 61 of the lead 40.

The constant-voltage output amplifier applies a voltage pulse to the distal electrode (cathode) 61 of the lead 40. A typical circuit diagram of a voltage output circuit is shown in FIG. 50. This configuration contains a stimulus amplitude generator 206 for generating an analog voltage. The analog voltage represents the stimulus amplitude and is stored on a holding capacitor Cₕ 225. Two switches are used to deliver the stimulus pulses to the lead 40, a stimulating delivery switch 220, and a recharge switch 222, that reestablishes the charge equilibrium after the stimulating pulse has been delivered to the nerve tissue. Since these switches have leakage currents that can cause direct current (DC) to flow into the lead system 40, a DC blocking capacitor C_{b} 229, is included. This is to prevent any possible corrosion that may result from the leakage of current in the lead 40. When the stimulus delivery switch 220 is closed, the pulse amplitude analog voltage stored in the (Cₕ 225) holding capacitor is transferred to the cathode electrode 61 of the lead 40 through the coupling capacitor, C_{b} 229. At the end of the stimulus pulse, the stimulus delivery switch 220 opens. The pulse duration being the interval from the closing of the switch 220 to its reopening. During the stimulus delivery, some of the charge stored on Cₕ 225 has been transferred to C_{b} 229, and some has been delivered to the lead system 40 to stimulate the nerve tissue.

To re-establish equilibrium, the recharge switch 222 is closed, and a rapid recharge pulse is delivered. This is intended to remove any residual charge remaining on the coupling capacitor C_{b} 229, and the stimulus electrodes on the lead (polarization). Thus, the stimulus is delivered as the result of closing and opening of the stimulus delivery 220 switch and the closing and opening of the RCHG switch 222. At this point, the charge on the holding Cₕ 225 must be replenished by the stimulus amplitude generator 206 before another stimulus pulse can be delivered.

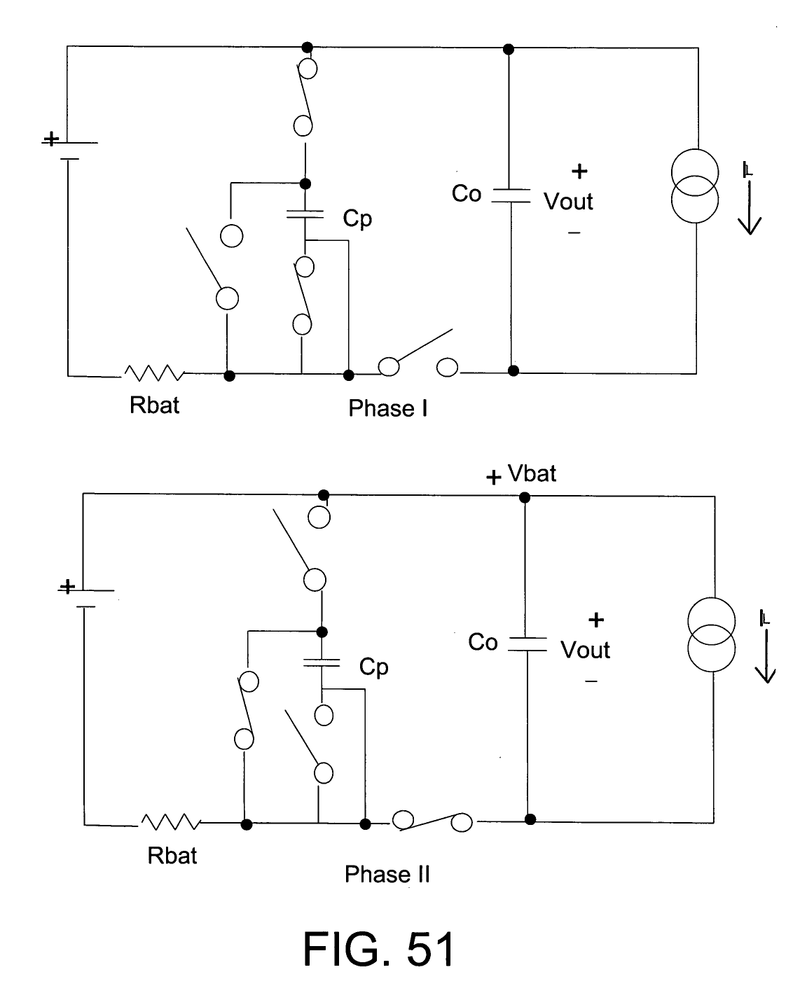

The pulse generating unit charges up a capacitor and the capacitor is discharged when the control (timing) circuitry requires the delivery of a pulse. This embodiment utilizes a constant voltage pulse generator, even though a constant current pulse generator can also be utilized. Pump-up capacitors are used to deliver pulses of larger magnitude than the potential of the batteries. The pump up capacitors are charged in parallel and discharged into the output capacitor in series. Shown in conjunction with FIG. 51 is a circuit diagram of a voltage doubler which is shown here as an example. For higher multiples of battery voltage, this doubling circuit can be cascaded with other doubling circuits. As shown in FIG. 51, during phase I (top of FIG. 51), the pump capacitor Cₚ is charged to V_{bat} and the output capacitor Cₒ supplies charge to the load. During phase II, the pump capacitor charges the output capacitor, which is still supplying the load current. In this case, the voltage drop across the output capacitor is twice the battery voltage.

FIG. 52A shows one example of the pulse trains that may be delivered with this embodiment or in prior art nerve stimulators. The microcontroller is configured to deliver the pulse train as shown in the figure, i.e. there is “ramping up” of the pulse train. The purpose of the ramping-up is to avoid sudden changes in stimulation, when the pulse train begins. The ramping-up or ramping-down is optional, and may be programmed into the microcontroller.

In the method and system of the current invention, the microcontroller is configured to deliver rectangular and complex pulses. Complex pulses comprise non-rectangular, biphasic, multi-step, and other complex pulses where the amplitude is varying during the pulse. Advantageously, these complex pulses provide a new dimension to selective stimulation or neuromodulation of cortical neural tissues to provide therapy for neurological disorders such as involuntary movement disorders, or for enhancing or inducing neuroplasticity.

Examples of these pulses and pulse trains are shown in FIGS. 52B to 52H. Selective stimulation with these complex pulses takes into account the threshold properties of different types of neural cells, as well as, their different refractory properties.

For example in the multi-step pulse shown in FIG. 52C, the first part of the pulse will tend to recruit large diameter (and myelinated) fibers. The middle portion of the pulse where the amplitude is highest, will tend to recruit different cells. Further, as shown in the examples of FIGS. 52F and 52H, complex and simple pulses, or pulse trains may be alternated.

The pulses and pulse trains of this disclosure gives physicians a lot of flexibility for trying various different neuromodulation algorithms, for providing and optimizing therapy for involuntary movement disorders, or for neuroplasticity.

Furthermore, as shown in conjunction with FIG. 52-I, a combination of tripolar electrodes with different pulse shapes may be used for selective stimulation of different types of nerve cells in the brain. The different pulses used in conjunction with tripolar electrodes are shown in conjunction with FIGS. 52J, 52K, 52L, 52M, 52N, and 52-O. This combination is advantageous, because it can be used to provide selective large fiber block as well.

The combination of tripolar electrodes and the pulse shapes of FIGS. 52-J to 52-O gives physicians more flexibility or providing stimulation therapy for their patients. In the tripolar electrodes (FIG. 52-I), the electrode consists of a cathode, flanked by two anodes. When stimulation is applied, the nerve membrane is depolarized near the cathode and hyperpolarized near the anodes. If the membrane is sufficiently hyperpolarized, an action potential (AP) that travels into the depolarized zone cannot pass the hyperpolarized zone and is arrested.

As shown in FIGS. 52J and 52K, the microcontroller 398 in the pulse generator 391 is configured to provide stepped pulses. The current of the first step is too low to induce an action potential (AP), but only depolarizes the membrane. The AP is generated during the second step. The pulses in FIGS. 52J and 52K are similar, except that the pulses in FIG. 52J have a longer first step. In addition to anodel blocking, another advantage of these stepped pulses is that the total charge per pulse can be reduced by almost a third.

Other examples of complex pulses, that may be used with tripolar electrodes are shown in FIGS. 52-L to 52-O. FIG. 52L shows biphasic pulses with a time delay t_{d} between the positive and negative pulse. FIG. 52M shows biphasic pulses with a time delay t_{d}, where the second part of the pulse is a step pulse. FIG. 52N shows ramp pulses, and FIG. 52-O show pulses with exponential components. Theoretical work, computer modeling, and animal studies have all shown that lower charge is obtained with these modified pulses when compared to square pulses. The charge reduction of these pulses can be approximately 30% less when compared to square pulses, which is fairly significant. The microcontroller 398 of the pulse generator 391 can be configured to deliver these pulses, as is well known to one skilled in the art.

Since the number of types of pulses and pulse trains to provide therapy can be complex for many physician's, in one aspect predetermined/pre-packaged program comprise a complete program for the pulse trains that deliver therapy. The advantage of the pre-packaged programs is that the physician may program a complicated program simply by selecting a program number.

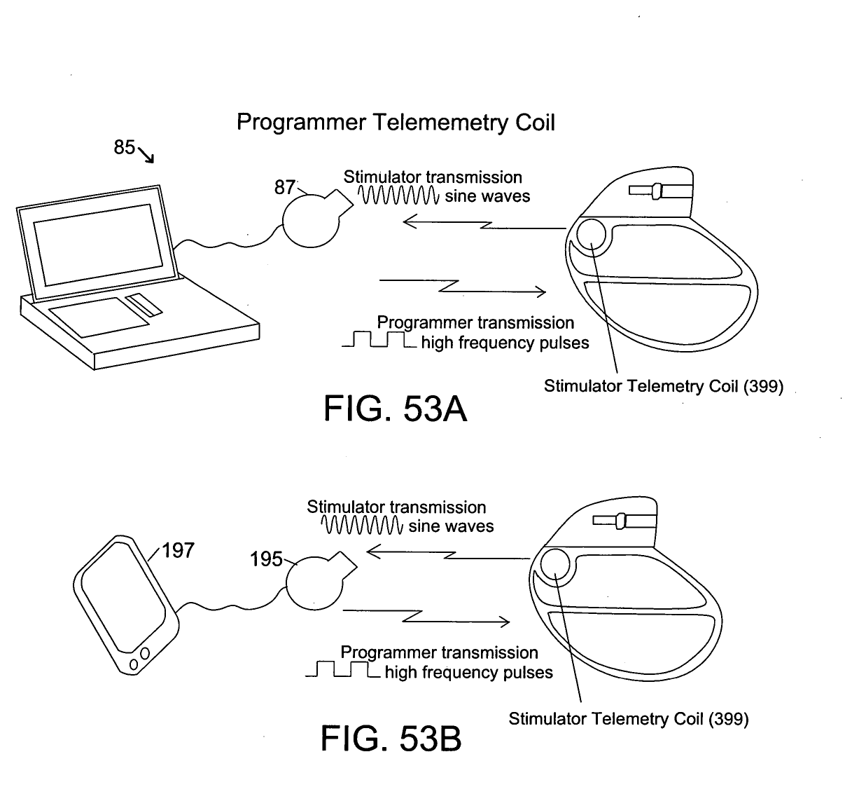

The programming of the implanted pulse generator (IPG) 391 is shown in conjunction with FIGS. 53A and 53B. With the magnetic Reed Switch 389 (FIG. 44A) in the closed position, a coil in the head of the programmer 85, communicates with a telemetry coil 399 of the implanted pulse generator 391. Bi-directional inductive telemetry is used to exchange data with the implanted unit 391 by means of the external programming unit 85.

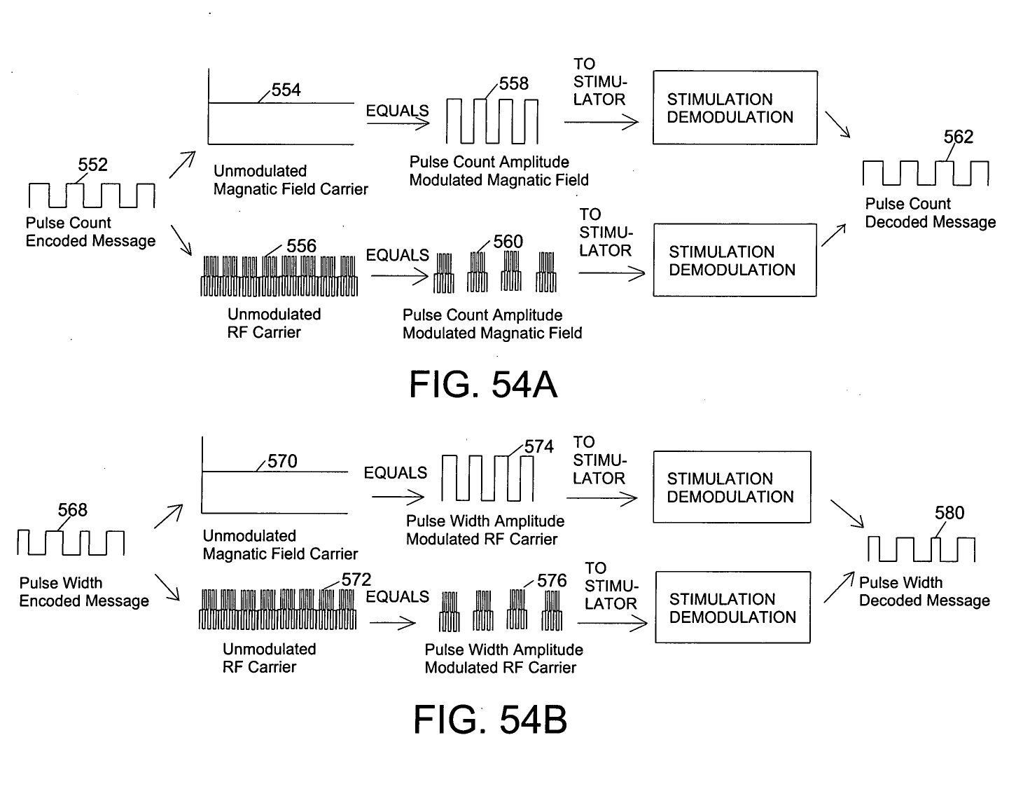

The transmission of programming information involves manipulation of the carrier signal in a manner that is recognizable by the pulse generator 391 as a valid set of instructions. The process of modulation serves as a means of encoding the programming instruction in a language that is interpretable by the implanted pulse generator 391. Modulation of signal amplitude, pulse width, and time between pulses are all used in the programming system, as will be appreciated by those skilled in the art. FIG. 54A shows an example of pulse count modulation, and FIG. 54B shows an example of pulse width modulation, that can be used for encoding.

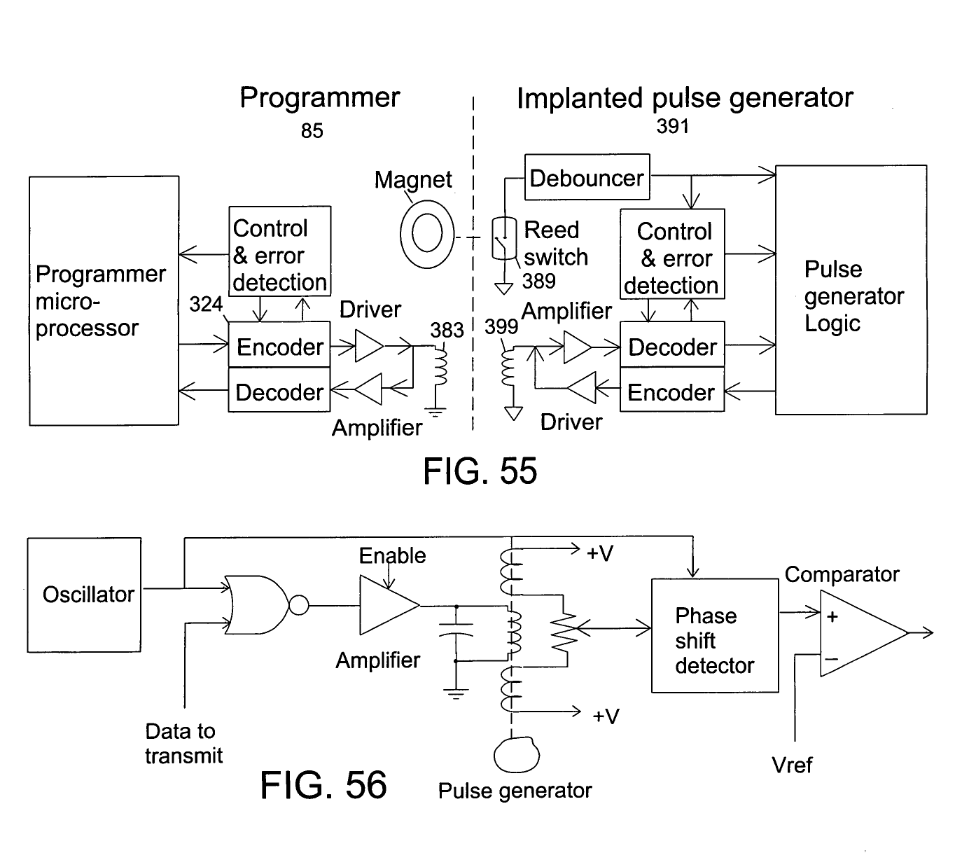

FIG. 55 shows a simplified overall block diagram of the implanted pulse generator (IPG) 391 programming and telemetry interface. The left half of FIG. 55 is programmer 85 which communicates programming and telemetry information with the IPG 391. The sections of the IPG 391 associated with programming and telemetry are shown on the right half of FIG. 55. In this case, the programming sequence is initiated by bringing a permanent magnet in the proximity of the IPG 391 which closes a reed switch 389 in the IPG 391. Information is then encoded into a special error-correcting pulse sequence and transmitted electromagnetically through a set of coils. The received message is decoded, checked for errors, and passed on to the unit's logic circuitry. The IPG 391 of this embodiment includes the capability of bidirectional communication.

The reed switch 389 is a magnetically-sensitive mechanical switch, which consists of two thin strips of metal (the “reed”) which are ferromagnetic. The reeds normally spring apart when no magnetic field is present. When a field is applied, the reeds come together to form a closed circuit because doing so creates a path of least reluctance. The programming head of the programmer contains a high-field-strength ceramic magnet.

When the switch closes, it activates the programming hardware, and initiates an interrupt of the IPG central processor. Closing the reed switch 389 also presents the logic used to encode and decode programming and telemetry signals. A nonmaskable interrupt (NMI) is sent to the IPG processor, which then executes special programming software. Since the NMI is an edge-triggered signal and the reed switch is vulnerable to mechanical bounce, a debouncing circuit is used to avoid multiple interrupts. The overall current consumption of the IPG increases during programming because of the debouncing circuit and other communication circuits.

A coil 399 is used as an antenna for both reception and transmission. Another set of coils 383 is placed in the programming head, a relatively small sized unit connected to the programmer 85. All coils are tuned to the same resonant frequency. The interface is half-duplex with one unit transmitting at a time.

Since the relative positions of the programming head 87 and IPG 391 determine the coupling of the coils, this embodiment utilizes a special circuit which has been devised to aid the positioning of the programming head, and is shown in FIG. 56. It operates on similar principles to the linear variable differential transformer. An oscillator tuned to the resonant frequency of the pacemaker coil 399 drives the center coil of a three-coil set in the programmer head. The phase difference between the original oscillator signal and the resulting signal from the two outer coils is measured using a phase shift detector. It is proportional to the distance between the implanted pulse generator and the programmer head. The phase shift, as a voltage, is compared to a reference voltage and is then used to control an indicator such as an LED. An enable signal allows switching the circuit on and off.

Actual programming is shown in conjunction with FIGS. 53 and 54. Programming and telemetry messages comprise many bits; however, the coil interface can only transmit one bit at a time. In addition, the signal is modulated to the resonant frequency of the coils, and must be transmitted in a relatively short period of time, and must provide detection of erroneous data.

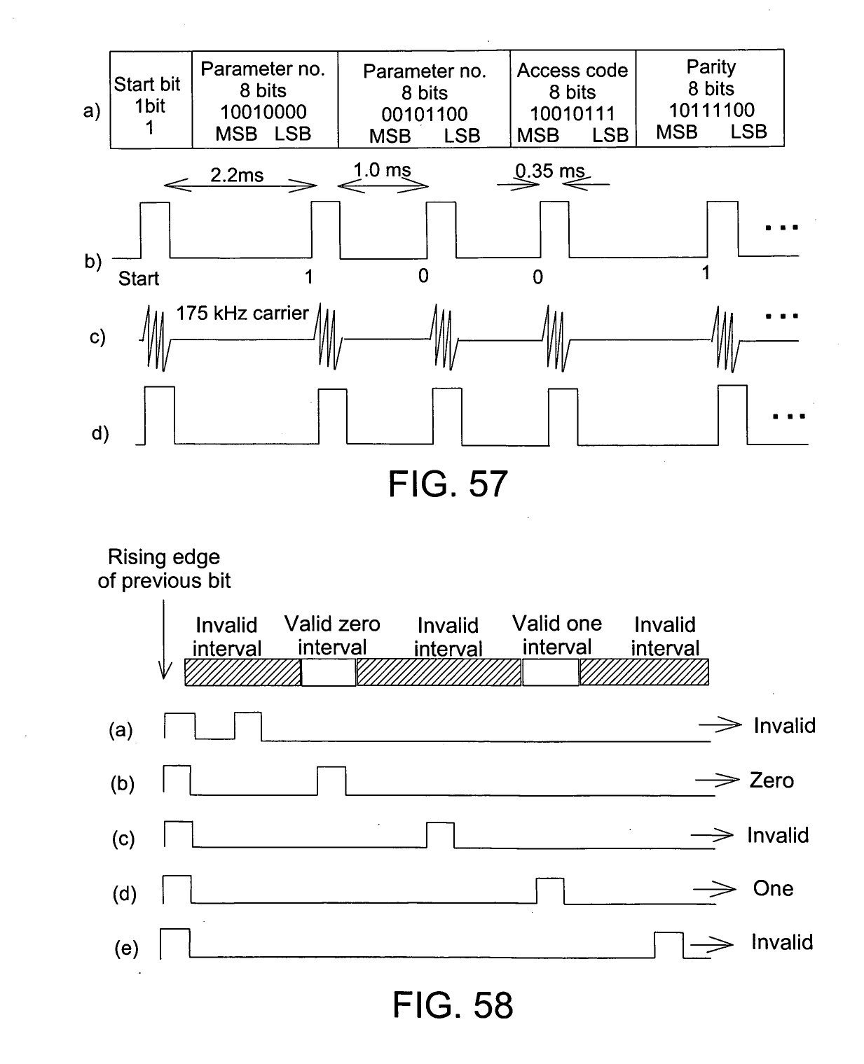

A programming message is comprised of five parts FIG. 57(a). The start bit indicates the beginning of the message and is used to synchronize the timing of the rest of the message. The parameter number specifies which parameter (e.g., mode, pulse width, delay) is to be programmed. In the example, in FIG. 57(a) the number 10010000 specifies the pulse rate to be specified. The parameter value represents the value that the parameter should be set to. This value may be an index into a table of possible values; for example, the value 00101100 represents a pulse stimulus rate of 80 pulses/min. The access code is a fixed number based on the stimulus generator model which must be matched exactly for the message to succeed. It acts as a security mechanism against use of the wrong programmer, errors in the message, or spurious programming from environmental noise. It can also potentially allow more than one programmable implant in the patient. Finally, the parity field is the bitwise exclusive-OR of the parameter number and value fields. It is one of several error-detection mechanisms.

All of the bits are then encoded as a sequence of pulses of 0.35-ms duration FIG. 57(b). The start bit is a single pulse. The remaining bits are delayed from their previous bit according to their bit value. If the bit is a zero, the delay is short (1.0); if it is a one, the delay is long (2.2 ms). This technique of pulse position coding, makes detection of errors easier.

The serial pulse sequence is then amplitude modulated for transmission FIG. 57(c). The carrier frequency is the resonant frequency of the coils. This signal is transmitted from one set of coils to the other and then demodulated back into a pulse sequence FIG. 57(d).

FIG. 58 shows how each bit of the pulse sequence is decoded from the demodulated signal. As soon as each bit is received, a timer begins timing the delay to the next pulse. If the pulse occurs within a specific early interval, it is counted as a zero bit (FIG. 58(b)). If it otherwise occurs with a later interval, it is considered to be a one bit (FIG. 58(d)). Pulses that come too early, too late, or between the two intervals are considered to be errors and the entire message is discarded (FIG. 58 (a, c, e)). Each bit begins the timing of the bit that follows it. The start bit is used only to time the first bit.

Telemetry data may be either analog or digital. Digital signals are first converted into a serial bit stream using an encoding such as shown in FIG. 58 (b). The serial stream or the analog data is then frequency modulated for transmission.

An advantage of this and other encodings is that they provide multiple forms of error detection. The coils and receiver circuitry are tuned to the modulation frequency, eliminating noise at other frequencies. Pulse-position coding can detect errors by accepting pulses only within narrowly-intervals. The access code acts as a security key to prevent programming by spurious noise or other equipment. Finally, the parity field and other checksums provides a final verification that the message is valid. At any time, if an error is detected, the entire message is discarded.

Another more sophisticated type of pulse position modulation may be used to increase the bit transmission rate. In this, the position of a pulse within a frame is encoded into one of a finite number of values, e.g. 16. A special synchronizing bit is transmitted to signal the start of the frame. Typically, the frame contains a code which specifies the type or data contained in the remainder of the frame.

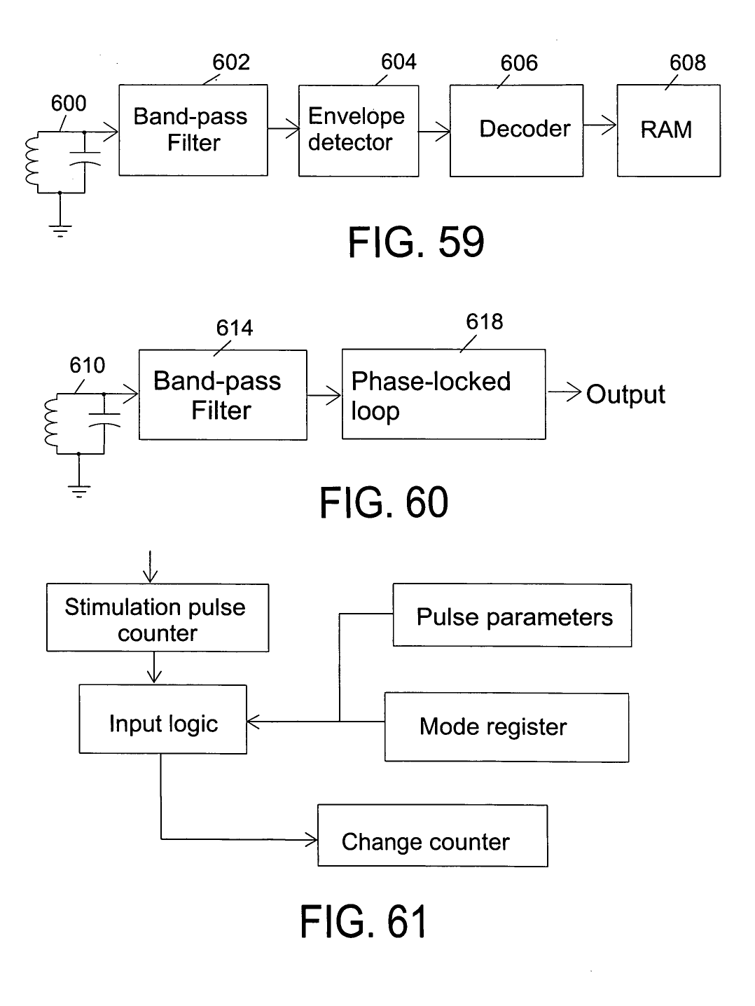

FIG. 59 shows a diagram of receiving and decoding circuitry for programming data. The IPG coil, in parallel with capacitor creates a tuned circuit for receiving data. The signal is band-pass filtered 602 and envelope detected 604 to create the pulsed signal in FIG. 57 (d). After decoding, the parameter value is placed in a RAM at the location specified by the parameter number. The IPG can have two copies of the RAM—a permanent set and a temporary set—which makes it easy for the physician to set the IPG to a temporary configuration and later reprogram it back to the usual settings.

FIG. 60 shows the basic circuit used to receive telemetry data. Again, a coil and capacitor create a resonant circuit tuned to the carrier frequency. The signal is further band-pass filtered 614 and then frequency-demodulated using a phase-locked loop 618.

This embodiment also comprises an optional battery status test circuit. Shown in conjunction with FIG. 61, the charge delivered by the battery is estimated by keeping track of the number of pulses delivered by the IPG 391. An internal charge counter is updated during each test mode to read the total charge delivered. This information about battery status is read from the IPG 391 via telemetry.

### Combination Implantable Device Comprising Both a Stimulus-Receiver and a Programmable Implantable Pulse Generator (IPG)

In one embodiment, the implantable device may comprise both a stimulus-receiver and a programmable implantable pulse generator (IPG) in one device. One such system is also disclosed in applicant's co-pending application Ser. No. 10/436,017 and is incorporated herein by reference. This embodiment also comprises predetermined/pre-packaged programs. Examples of several stimulation states were given in the previous sections, under “Programmer-less Implantable Pulse Generator (IPG)” and “Programmable Implantable Pulse Generator”. These predetermined/pre-packaged programs comprise unique combinations of pulse amplitude, pulse width, pulse frequency, ON-time and OFF-time.

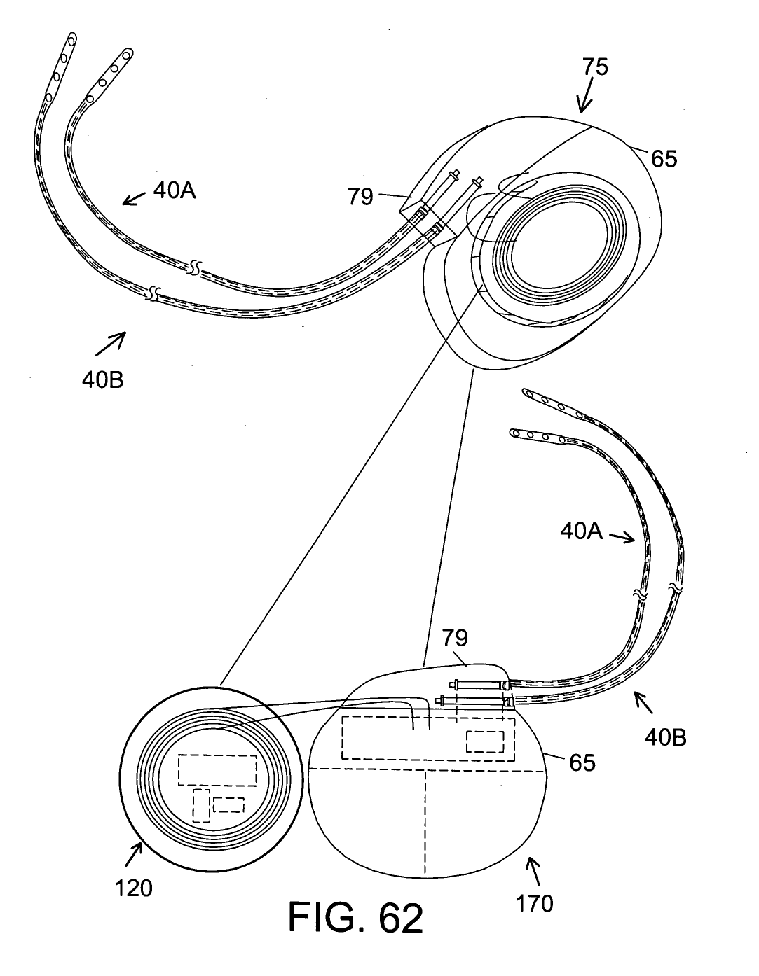

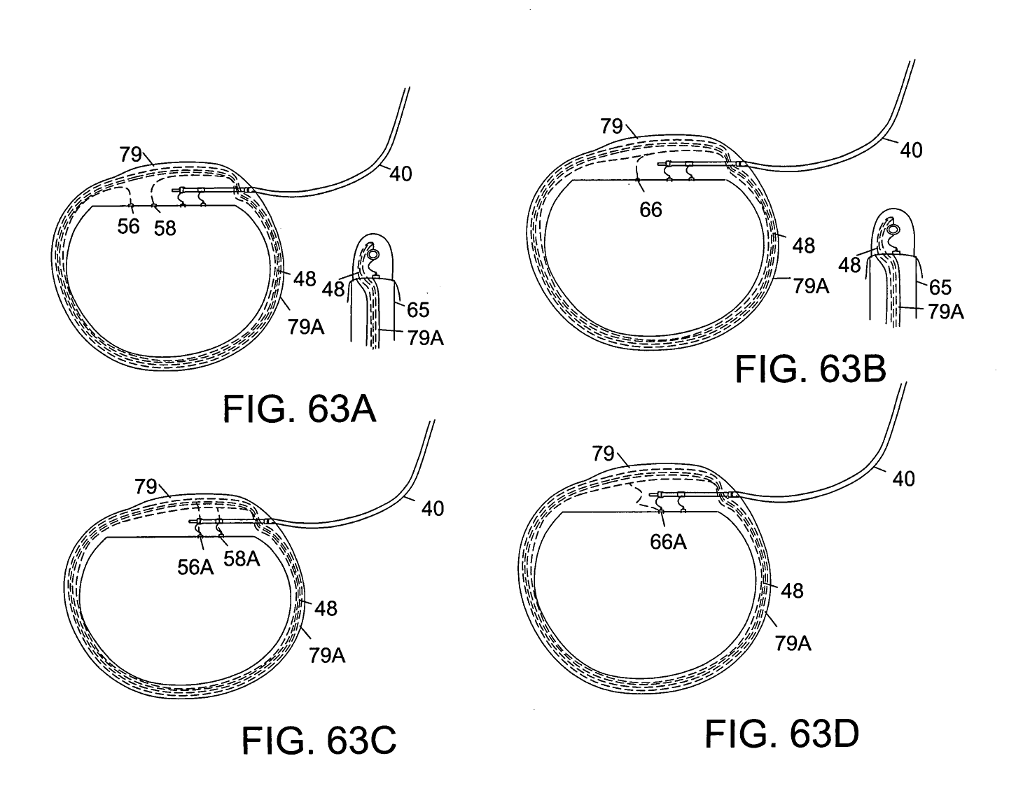

FIG. 62 shows a close up view of the packaging of the implanted stimulator 75 of this embodiment, showing the two subassemblies 120, 170. The two subassemblies are the stimulus-receiver module 120 and the battery operated pulse generator module 170. The electrical components of the stimulus-receiver module 120 may be substantially in the titanium case along with other circuitry, except for a coil. The coil may be outside the titanium case as shown in FIG. 62, or the coil 48C may be externalized at the header portion 79 of the implanted device, and may be wrapped around the titanium can. In this case, the coil is encased in the same material as the header 79, as shown in FIGS. 63A-63D. FIG. 63A depicts a bipolar configuration with two separate feed-throughs, 56, 58. FIG. 63B depicts a unipolar configuration with one separate feed-through 66. FIG. 63C, and 63D depict the same configuration except the feed-throughs are common with the feed-throughs 66A for the lead.

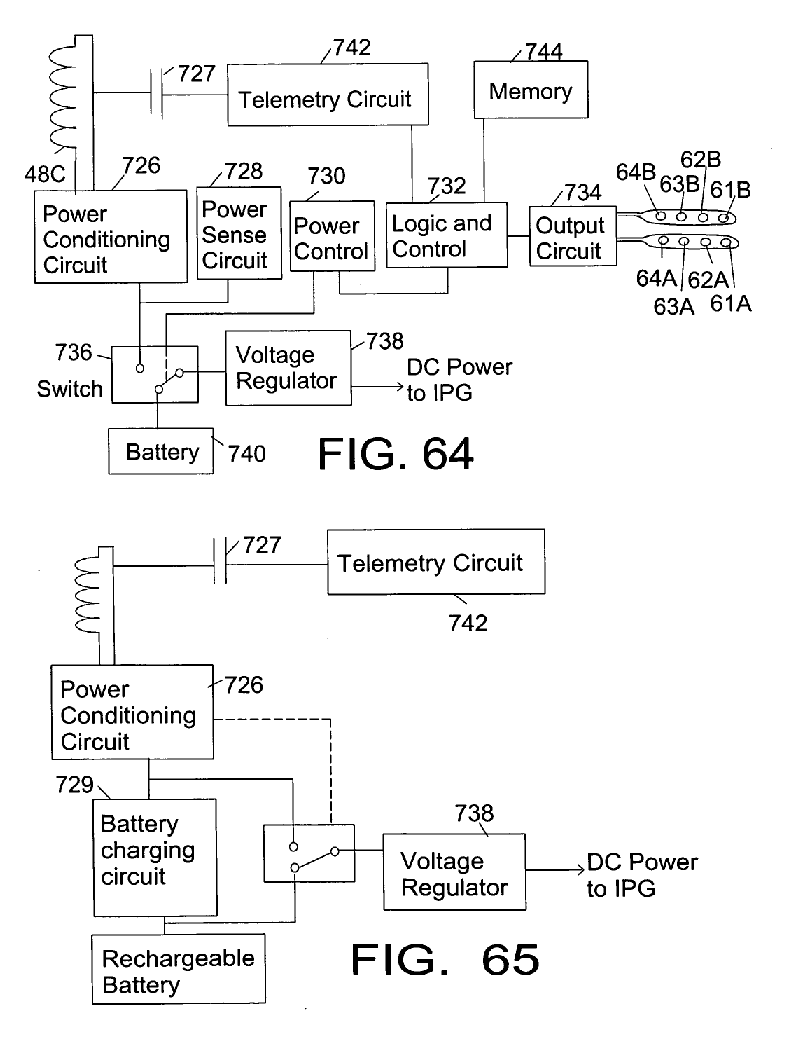

FIG. 64 is a simplified overall block diagram of the embodiment where the implanted stimulator 75 is a combination device, which may be used as a stimulus-receiver (SR) in conjunction with an external stimulator, or the same implanted device may be used as a traditional programmable implanted pulse generator (IPG). The coil 48C which is external to the titanium case may be used both as a secondary of a stimulus-receiver, or may also be used as the forward and back telemetry coil.

In this embodiment, as disclosed in FIG. 64, the IPG circuitry within the titanium case is used for all stimulation pulses whether the energy source is the internal battery 740 or an external power source. The external device serves as a source of energy, and as a programmer that sends telemetry to the IPG. For programming, the energy is sent as high frequency sine waves with superimposed telemetry wave driving the external coil 46C. Once received by the implanted coil 48C, the telemetry is passed through coupling capacitor 727 to the IPG's telemetry circuit 742. For pulse delivery using external power source, the stimulus-receiver portion will receive the energy coupled to the implanted coil 48C and, using the power conditioning circuit 726, rectify it to produce DC, filter and regulate the DC, and couple it to the IPG's voltage regulator 738 section so that the IPG can run from the externally supplied energy rather than the implanted battery 740.

The system provides a power sense circuit 728 that senses the presence of external power communicated with the power control 730 when adequate and stable power is available from an external source. The power control circuit controls a switch 736 that selects either battery power 740 or conditioned external power from 726. The logic and control section 732 and memory 744 includes the IPG's microcontroller, pre-programmed instructions, and stored chagneable parameters. Using input for the telemetry circuit 742 and power control 730, this section controls the output circuit 734 that generates the output pulses.

It will be clear to one skilled in the art that this embodiment of the invention can also be practiced with a rechargeable battery. This version is shown in conjunction with FIG. 65. The circuitry in the two versions are similar except for the battery charging circuitry 749. This circuit is energized when external power is available. It senses the charge state of the battery and provides appropriate charge current to safely recharge the battery without overcharging.

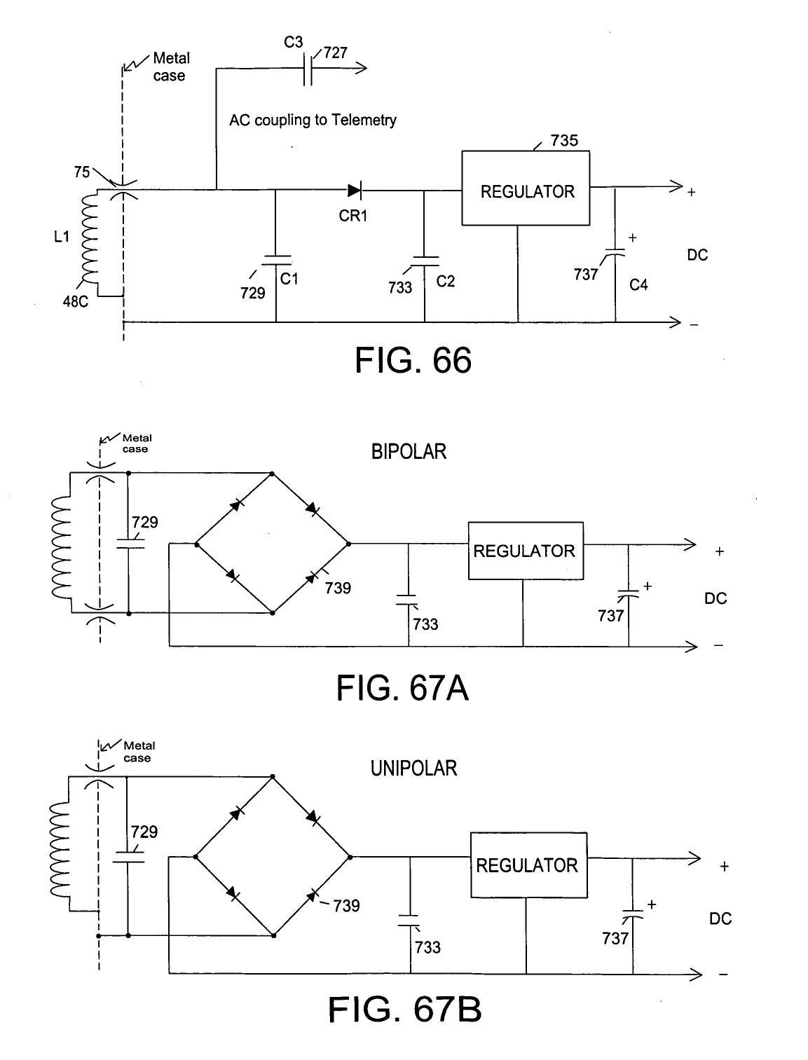

The stimulus-receiver portion of the circuitry is shown in conjunction with FIG. 66. Capacitor C1 (729) makes the combination of C1 and L1 sensitive to the resonant frequency and less sensitive to other frequencies, and energy from an external (primary) coil 46C is inductively transferred to the implanted unit via the secondary coil 48C. The AC signal is rectified to DC via diode 731, and filtered via capacitor 733. A regulator 735 sets the output voltage and limits it to a value just above the maximum IPG cell voltage. The output capacitor C4 (737), typically a tantalum capacitor with a value of 100 micro-Farads or greater, stores charge so that the circuit can supply the IPG with high values of current for a short time duration with minimal voltage change during a pulse while the current draw from the external source remains relatively constant. Also shown in conjunction with FIG. 66, a capacitor C3 (727) couples signals for forward and back telemetry.

FIGS. 67A and 67B show alternate connection of the receiving coil. In FIG. 67A, each end of the coil is connected to the circuit through a hermetic feedthrough filter. In this instance, the DC output is floating with respect to the IPG's case. In FIG. 67B, one end of the coil is connected to the exterior of the IPG's case. The circuit is completed by connecting the capacitor 729 and bridge rectifier 739 to the interior of the IPG's case The advantage of this arrangement is that it requires one less hermetic feedthrough filter, thus reducing the cost and improving the reliabilty of the IPG. Hermetic feedthrough filters are expensive and a possible failure point. However, the case connection may complicit the output circuitry or limit its versatility. When using a bipolar electrode (or multipolar electrodes), care must be taken to prevent an unwanted return path for the pulse to the IPG's case. This is not a concern for unipolar pulses using a single conductor electrode because it relies on the IPG's case a return for the pulse current.

In the unipolar configuration, a bigger tissue area is stimulated since the difference between the tip (cathode) and case (anode) is larger. Stimulations using unipolar, bipolar, and multipolar configurations is considered within the scope of this invention.

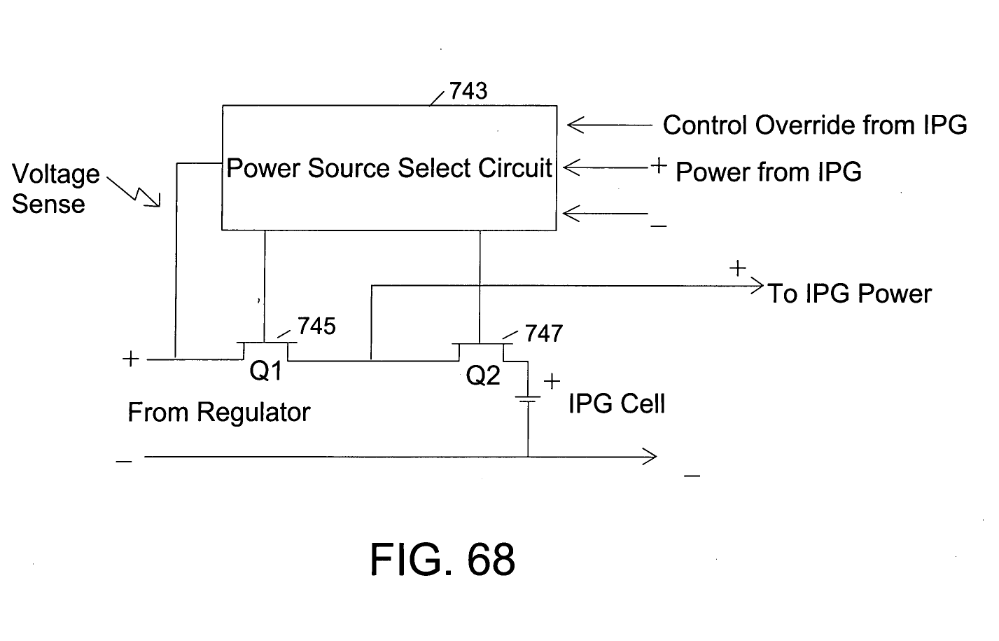

The power source select circuit is highlighted in conjunction with FIG. 68. In this embodiment, the IPG provides stimulation pulses according to the stimulation programs stored in the memory 744 of the implanted stimulator, with power being supplied by the implanted battery 740. When stimulation energy from an external stimulator is inductively received via secondary coil 48C, the power source select circuit (shown in block 743) switches power via transistor Q1 745 and transistor Q2 743. Transistor Q1 and Q2 are preferably low loss MOS transistor used as switches, even though other types of transistors may be used.

### Implantable Pulse Generator (IPG) Comprising a Rechargable Battery

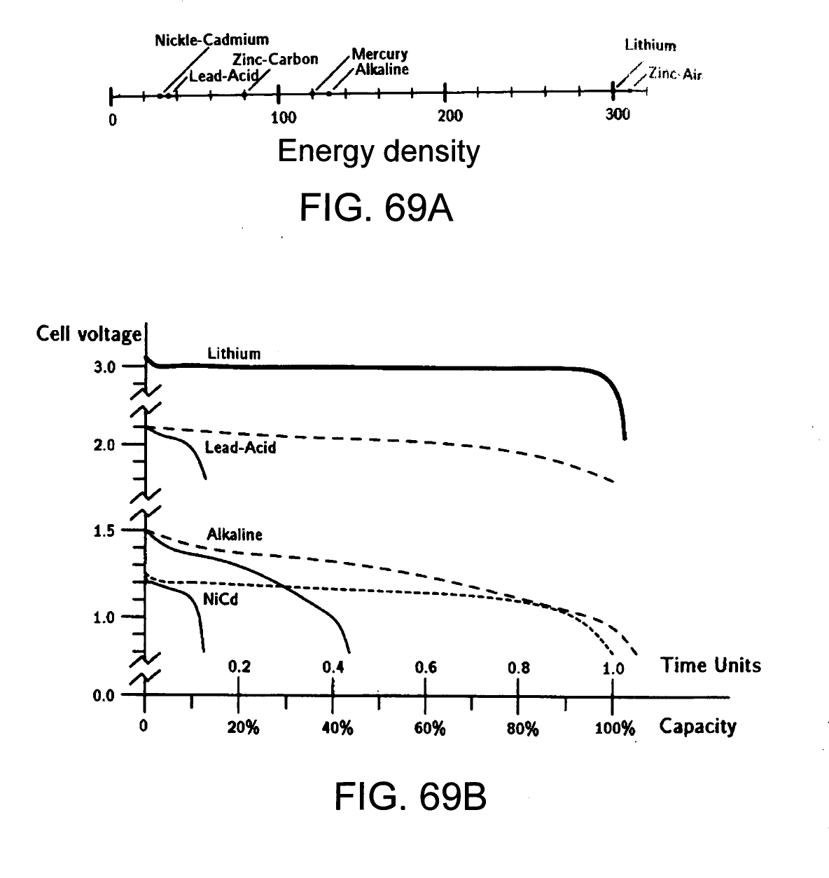

In one embodiment, an implantable pulse generator with rechargeable power source can be used. Because of the rapidity of the pulses required for modulating nerve tissue 54 with stimulating and/or blocking pulses, there is a real need for power sources that will provide an acceptable service life under conditions of continuous delivery of high frequency pulses. FIG. 69A shows a graph of the energy density of several commonly used battery technologies. Lithium batteries have by far the highest energy density of commonly available batteries. Also, a lithium battery maintains a nearly constant voltage during discharge. This is shown in conjunction with FIG. 69B, which is normalized to the performance of the lithium battery. Lithium-ion batteries also have a long cycle life, and no memory effect. However, Lithium-ion batteries are not as tolerant to overcharging and over-discharging. One of the most recent development in rechargable battery technology is the Lithium-ion polymer battery. Recently the major battery manufacturers (Sony, Panasonic, Sanyo) have announced plans for Lithium-ion polymer battery production. In one embodiment, existing nerve stimulators and cardiac pacemakers can be modified to incorporate rechargeable batteries.

This embodiment also comprises predetermined/pre-packaged programs. Examples of several stimulation states were given in the previous sections, under “Programmer-less Implantable Pulse Generator (IPG)” and “Programmable Implantable Pulse Generator”. These pre-packaged/pre-determined programs comprise unique combinations of pulse amplitude, pulse width, pulse frequency, ON-time and OFF-time. Additionally, predetermined programs comprising blocking pulses may also be stored in the memory of the pulse generator.

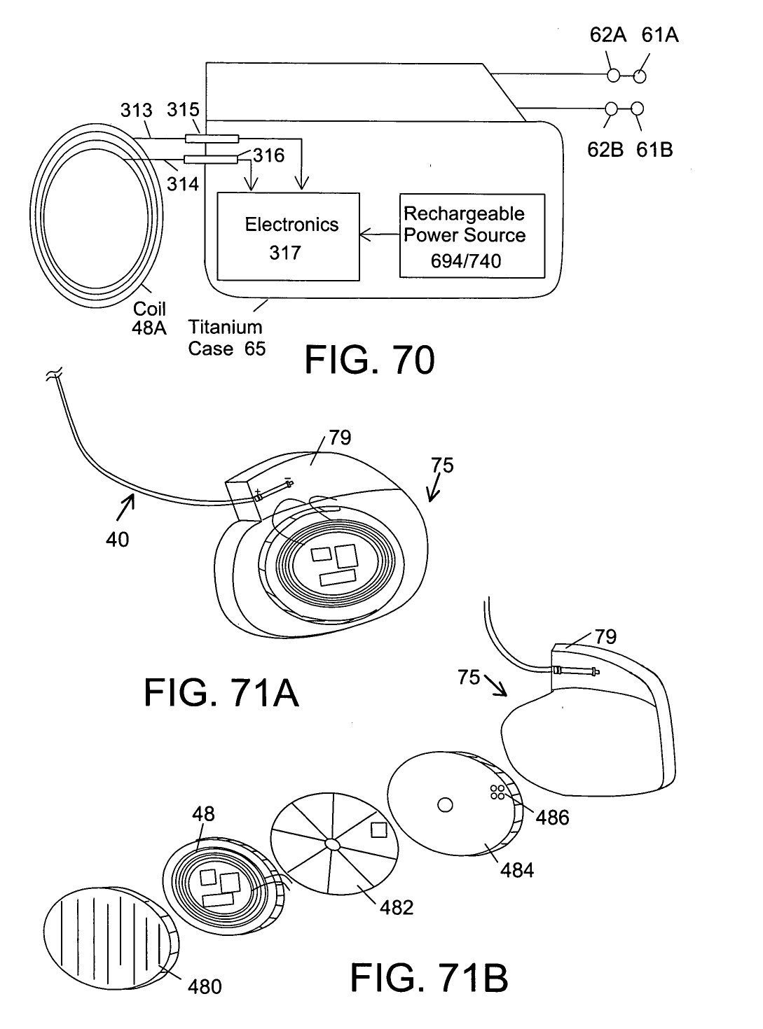

As shown in conjunction with FIG. 70, the coil is externalized from the titanium case 65. The RF pulses transmitted via coil 46 and received via subcutaneous coil 48A are rectified via a diode bridge. These DC pulses are processed and the resulting current applied to recharge the battery 694/740 in the implanted pulse generator. In one embodiment the coil 48C may be externalized at the header portion 79 of the implanted device, and may be wrapped around the titanium can, as was previously shown in FIGS. 63A-D.

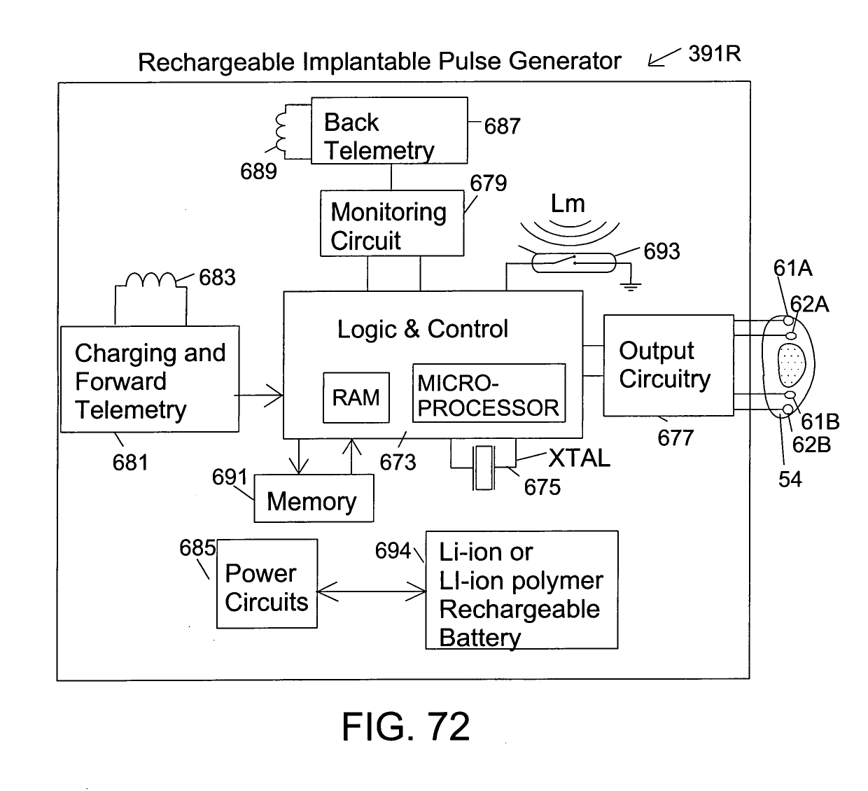

In one embodiment, the coil may also be positioned on the titanium case as shown in conjunction with FIGS. 71A and 71B. FIG. 71A shows a diagram of the finished implantable stimulator 391R of one embodiment. FIG. 72B shows the pulse generator with some of the components used in assembly in an exploded view. These components include a coil cover 480, the secondary coil 48 and associated components, a magnetic shield 482, and a coil assembly carrier 484. The coil assembly carrier 484 has at least one positioning detail 486 located between the coil assembly and the feed through for positioning the electrical connection. The positioning detail 486 secures the electrical connection.

A schematic diagram of the implanted pulse generator (IPG 391R), with re-chargeable battery 694, is shown in conjunction with FIG. 72. The IPG 391R includes logic and control circuitry 673 connected to memory circuitry 691. The operating program and stimulation parameters are typically stored within the memory 691 via forward telemetry. Stimulation pulses are provided to the nerve tissue 54 via output circuitry 677 controlled by the microcontroller.

The operating power for the IPG 391R is derived from a rechargeable power source 694. The rechargeable power source 694 comprises a rechargeable lithium-ion or lithium-ion polymer battery. Recharging occurs inductively from an external charger to an implanted coil 48B underneath the skin 60. The rechargeable battery 694 may be recharged repeatedly as needed. Additionally, the IPG 391R is able to monitor and telemeter the status of its rechargable battery 691 each time a communication link is established with the external programmer 85.

Much of the circuitry included within the IPG 391R may be realized on a single application specific integrated circuit (ASIC). This allows the overall size of the IPG 391R to be quite small, and readily housed within a suitable hermetically-sealed case. The IPG case is preferably made from a titanium and is shaped in a rounded case.

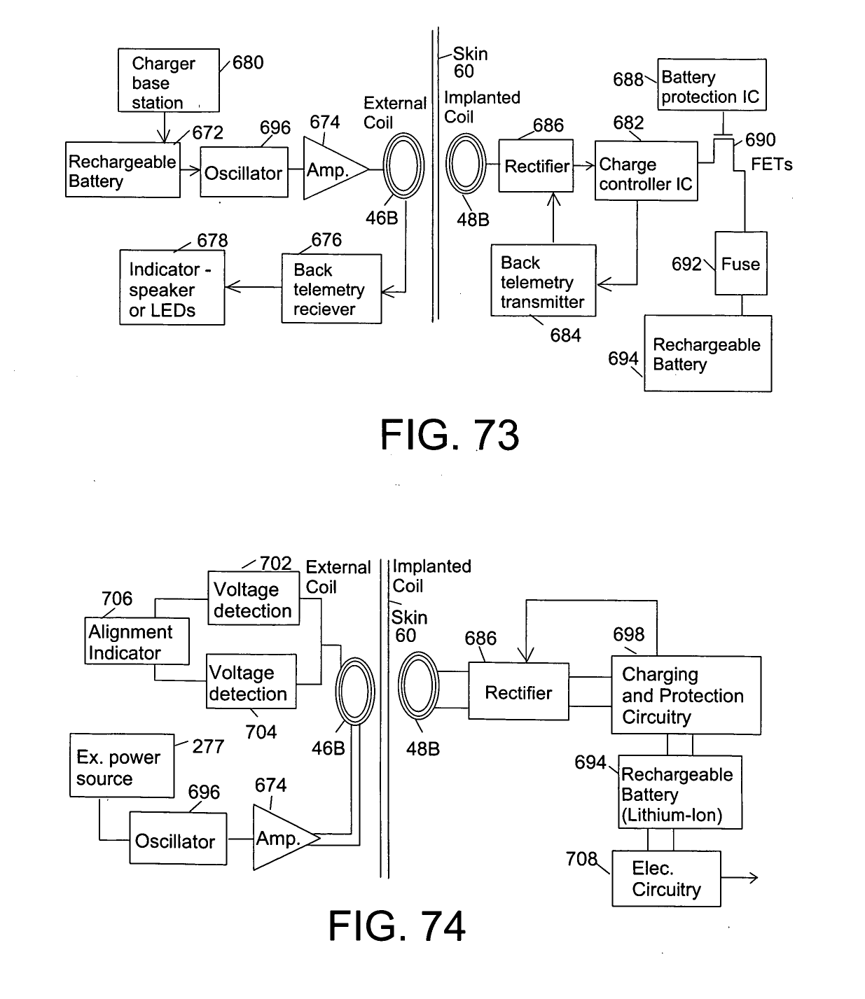

Shown in conjunction with FIG. 73 are the recharging elements of this embodiment. The re-charging system uses a portable external charger to couple energy into the power source of the IPG 391R. The DC-to-AC conversion circuitry 696 of the re-charger receives energy from a battery 672 in the re-charger. A charger base station 680 and conventional AC power line may also be used. The AC signals amplified via power amplifier 674 are inductively coupled between an external coil 46B and an implanted coil 48B located subcutaneously with the implanted pulse generator (IPG) 391R. The AC signal received via implanted coil 48B is rectified 686 to a DC signal which is used for recharging the rechargeable battery 694 of the IPG, through a charge controller IC 682. Additional circuitry within the IPG 391R includes, battery protection IC 688 which controls a FET switch 690 to make sure that the rechargeable battery 694 is charged at the proper rate, and is not overcharged. The battery protection IC 688 can be an off-the-shelf IC available from Motorola (part no. MC 33349N-3R1). This IC monitors the voltage and current of the implanted rechargeable battery 694 to ensure safe operation. If the battery voltage rises above a safe maximum voltage, the battery protection IC 688 opens charge enabling FET switches 690, and prevents further charging. A fuse 692 acts as an additional safeguard, and disconnects the battery 694 if the battery charging current exceeds a safe level. As

also shown in FIG. 73, charge completion detection is achieved by a back-telemetry transmitter 684, which modulates the secondary load by changing the full-wave rectifier into a half-wave rectifier/voltage clamp. This modulation is in turn, sensed by the charger as a change in the coil voltage due to the change in the reflected impedance. When detected through a back telemetry receiver 676, either an audible alarm is generated or a LED is turned on.

A simplified block diagram of charge completion and misalignment detection circuitry is shown in conjunction with FIG. 74. As shown, a switch regulator 686 operates as either a full-wave rectifier circuit or a half-wave rectifier circuit as controlled by a control signal (CS) generated by charging and protection circuitry 698. The energy induced in implanted coil 48B (from external coil 46B) passes through the switch rectifier 686 and charging and protection circuitry 698 to the implanted rechargeable battery 694. As the implanted battery 694 continues to be charged, the charging and protection circuitry 698 continuously monitors the charge current and battery voltage. When the charge current and battery voltage reach a predetermined level, the charging and protection circuitry 698 triggers a control signal. This control signal causes the switch rectifier 686 to switch to half-wave rectifier operation. When this change happens, the voltage sensed by voltage detector 702 causes the alignment indicator 706 to be activated. This indicator 706 may be an audible sound or a flashing LED type of indicator.

The indicator 706 may similarly be used as a misalignment indicator. In normal operation, when coils 46B (external) and 48B (implanted) are properly aligned, the voltage Vₛ sensed by voltage detector 704 is at a minimum level because maximum energy transfer is taking place. If and when the coils 46B and 48B become misaligned, then less than a maximum energy transfer occurs, and the voltage Vₛ sensed by detection circuit 704 increases significantly. If the voltage Vₛ reaches a predetermined level, alignment indicator 706 is activated via an audible speaker and/or LEDs for visual feedback. After adjustment, when an optimum energy transfer condition is established, causing Vₛ to decrease below the predetermined threshold level, the alignment indicator 706 is turned off.

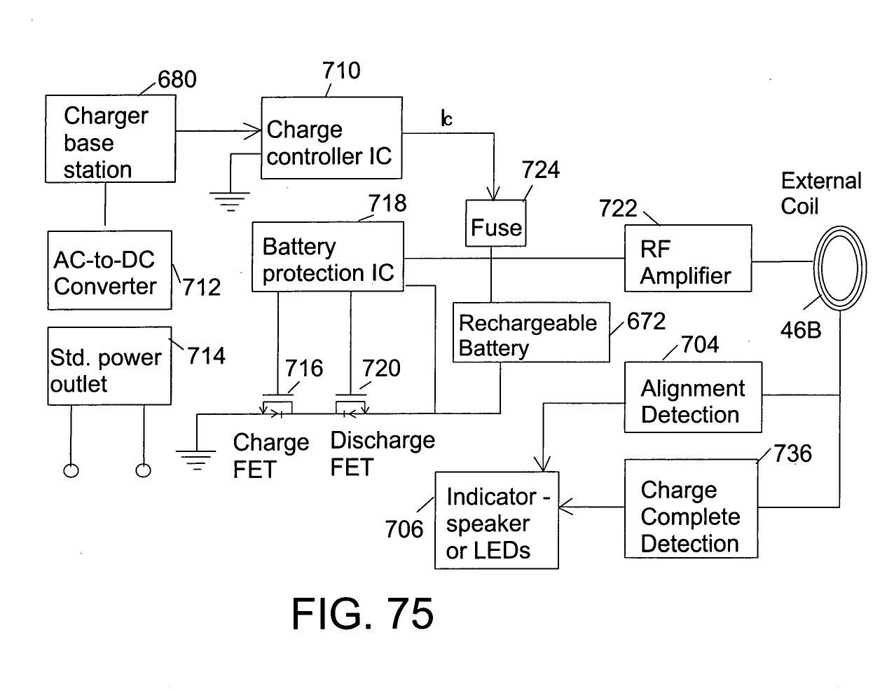

The elements of the external recharger are shown as a block diagram in conjunction with FIG. 75. In this disclosure, the words charger and recharger are used interchangeably. The charger base station 680 receives its energy from a standard power outlet 714, which is then converted to 5 volts DC by a AC-to-DC transformer 712. When the re-charger is placed in a charger base station 680, the re-chargeable battery 672 of the re-charger is fully recharged in a few hours and is able to recharge the battery 694 of the IPG

391R. If the battery 672 of the external re-charger falls below a prescribed limit of 2.5 volt DC, the battery 672 is trickle charged until the voltage is above the prescribed limit, and then at that point resumes a normal charging process.

As also shown in FIG. 75, a battery protection circuit 718 monitors the voltage condition, and disconnects the battery 672 through one of the FET switches 716, 720 if a fault occurs until a normal condition returns. A fuse 724 will disconnect the battery 672 should the charging or discharging current exceed a prescribed amount.

In summary, in the method of the current invention for neuromodulation of cortical neural tissue to provide therapy or alleviate symptoms of Parkinson's disease, or to enhance or induce neuroplasticity in post-stroke patients can be practiced with any of the several pulse generator systems disclosed including,

a) an implanted stimulus-receiver with an external stimulator;

b) an implanted stimulus-receiver comprising a high value capacitor for storing charge, used in conjunction with an external stimulator;

c) a programmer-less implantable pulse generator (IPG) which is operable with a magnet;

d) a microstimulator;

e) a programmable implantable pulse generator;

f) a combination implantable device comprising both a stimulus-receiver and a programmable IPG; and

g) an IPG comprising a rechargeable battery.

Electrical stimulation of cortical neural tissues with any of these embodiments is considered within the scope of this disclosure.

In one embodiment, the external stimulator and/or the programmer has a telecommunications module, as described in a co-pending application, and summarized here for reader convenience. The telecommunications module has two-way communications capabilities.

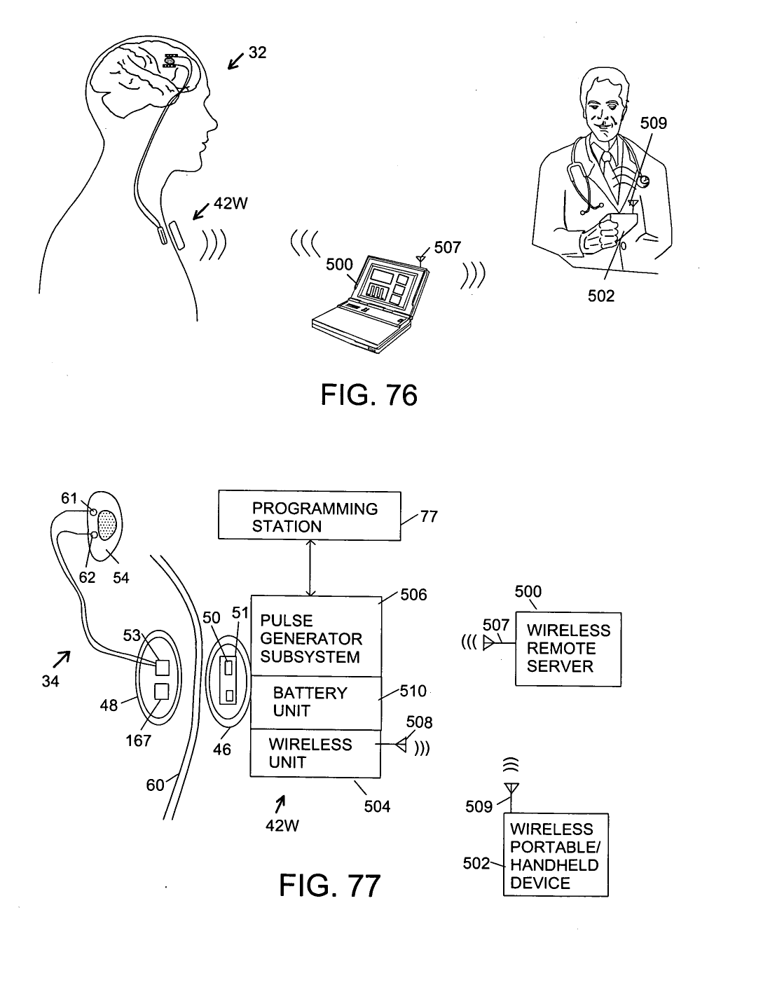

FIGS. 76 and 77 depict communication between an external stimulator 42 and a remote hand-held computer 502. A desktop or laptop computer can be a server 500 which is situated remotely, perhaps at a physician's office or a hospital. The stimulation parameter data can be viewed at this facility or reviewed remotely by medical personnel on a hand-held personal data assistant (PDA) 502, such as a “palm-pilot” from PALM corp. (Santa Clara, Calif.), a “Visor” from Handspring Corp. (Mountain view, CA) or on a personal computer (PC). The physician or appropriate medical personnel, is able to interrogate the external stimulator 42 device and know what the device is currently programmed to, as well as, get a graphical display of the pulse train. The wireless communication with the remote server 500 and hand-held PDA 502 would be supported in all geographical locations within and outside the United States (US) that provides cell phone voice and data communication service.

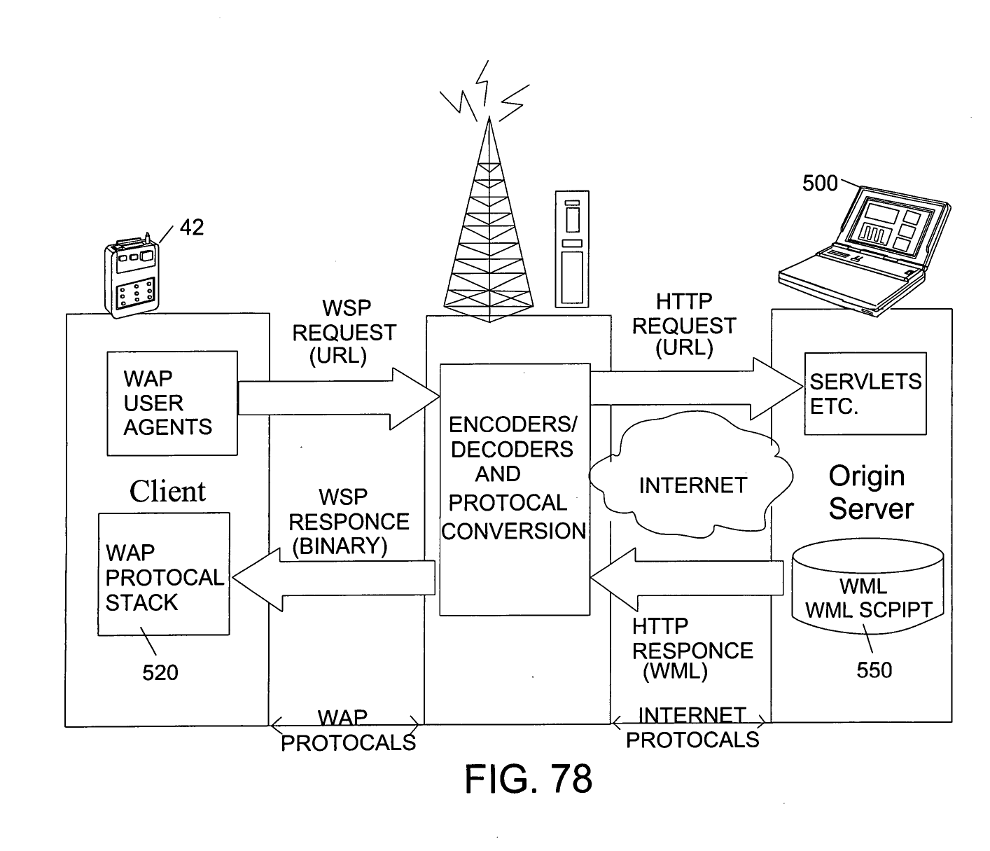

In one aspect of the invention, the telecommunications component can use Wireless Application Protocol (WAP). The Wireless Application Protocol (WAP), which is a set of communication protocols standardizing Internet access for wireless devices. While previously, manufacturers used different technologies to get Internet on hand-held devices, with WAP devices and services interoperate. WAP also promotes convergence of wireless data and the Internet. The WAP programming model is heavily based on the existing Internet programming model, and is shown schematically in FIG. 78. Introducing a gateway function provides a mechanism for optimizing and extending this model to match the characteristics of the wireless environment. Over-the-air traffic is minimized by binary encoding/decoding of Web pages and readapting the Internet Protocol stack to accommodate the unique characteristics of a wireless medium such as call drops.

The key components of the WAP technology, as shown in FIG. 78, includes 1) Wireless Mark-up Language (WML) 550 which incorporates the concept of cards and decks, where a card is a single unit of interaction with the user. A service constitutes a number of cards collected in a deck. A card can be displayed on a small screen. WML supported Web pages reside on traditional Web servers. 2) WML Script which is a scripting language, enables application modules or applets to be dynamically transmitted to the client device and allows the user interaction with these applets. 3) Microbrowser, which is a lightweight application resident on the wireless terminal that controls the user interface and interprets the WML/WMLScript content. 4) A lightweight protocol stack 520 which minimizes bandwidth requirements, guaranteeing that a broad range of wireless networks can run WAP applications. The protocol stack of WAP can comprise a set of protocols for the transport (WTP), session (WSP), and security (WTLS) layers. WSP is binary encoded and able to support header caching, thereby economizing on bandwidth requirements. WSP also compensates for high latency by allowing requests and responses to be handled asynchronously, sending before receiving the response to an earlier request. For lost data segments, perhaps due to fading or lack of coverage, WTP only retransmits lost segments using selective retransmission, thereby compensating for a less stable connection in wireless. The above mentioned features are industry standards adopted for wireless applications and greater details have been publicized, and well known to those skilled in the art.

In this embodiment, two modes of communication are possible. In the first, the server initiates an upload of the actual parameters being applied to the patient, receives these from the stimulator, and stores these in its memory, accessible to the authorized user as a dedicated content driven web page. The physician or authorized user can make alterations to the actual parameters, as available on the server, and then initiate a communication session with the stimulator device to download these parameters.

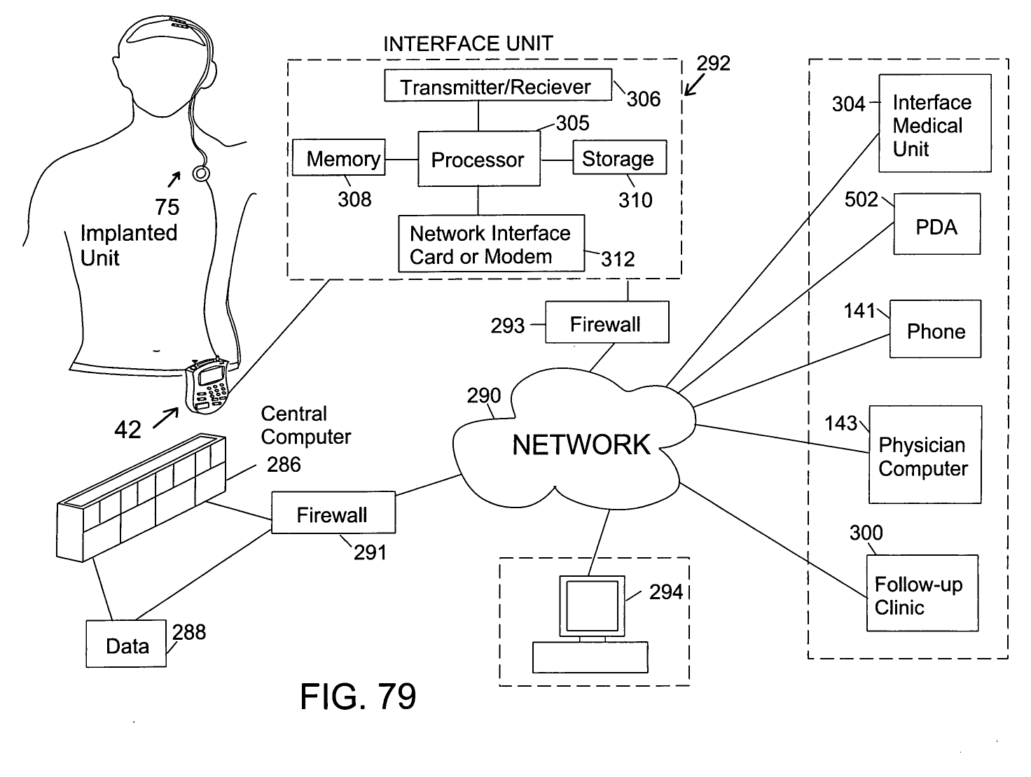

Shown in conjunction with FIG. 79, in one embodiment, the external stimulator 42 and/or the programmer 85 may also be networked to a central collaboration computer 286 as well as other devices such as a remote computer 294, PDA 502, phone 141, physician computer 143. The interface unit 292 in this embodiment communicates with the central collaborative network 290 via land-lines such as cable modem or wirelessly via the internet. A central computer 286 which has sufficient computing power and storage capability to collect and process large amounts of data, contains information regarding device history and serial number, and is in communication with the network 290. Communication over collaboration network 290 may be effected by way of a TCP/IP connection, particularly one using the internet, as well as a PSTN, DSL, cable modem, LAN, WAN or a direct dial-up connection.

The standard components of interface unit shown in block 292 are processor 305, storage 310, memory 308, transmitter/receiver 306, and a communication device such as network interface card or modem 312. In the preferred embodiment these components are embedded in the external stimulator 42 and can also be embedded in the programmer 85. These can be connected to the network 290 through appropriate security measures (Firewall) 293.

Another type of remote unit that may be accessed via central collaborative network 290 is remote computer 294. This remote computer 294 may be used by an appropriate attending physician to instruct or interact with interface unit 292, for example, instructing interface unit 292 to send instruction downloaded from central computer 286 to remote implanted unit.

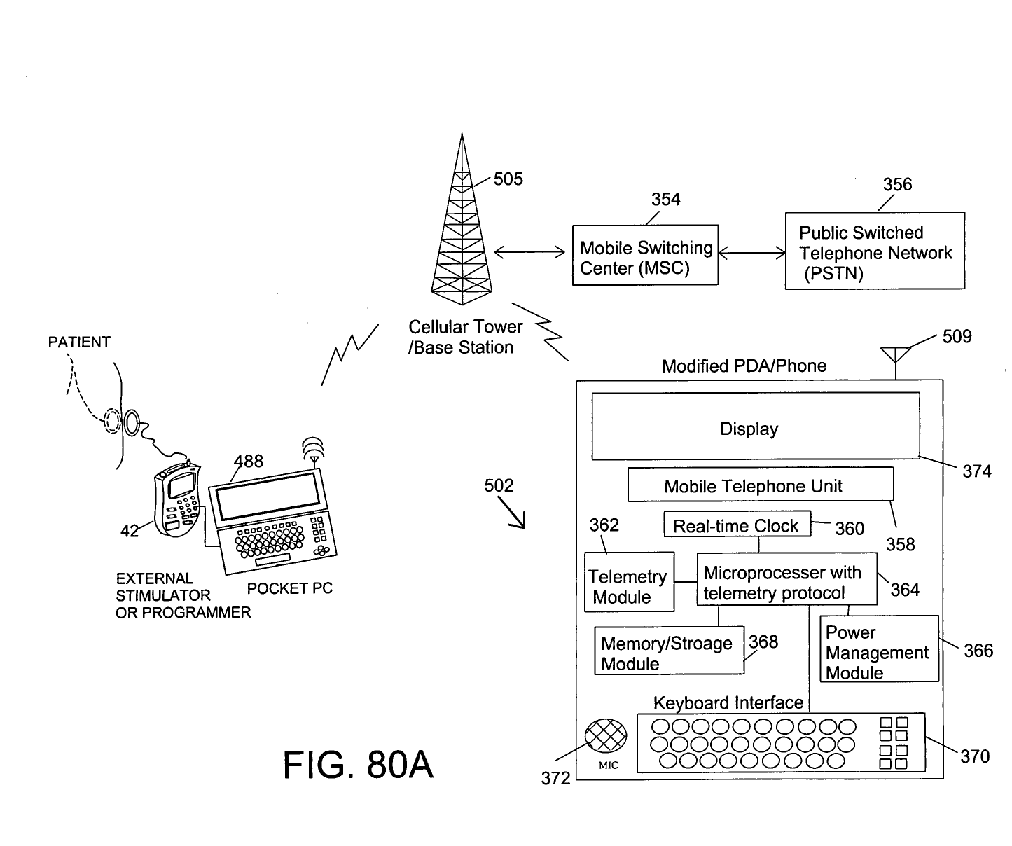

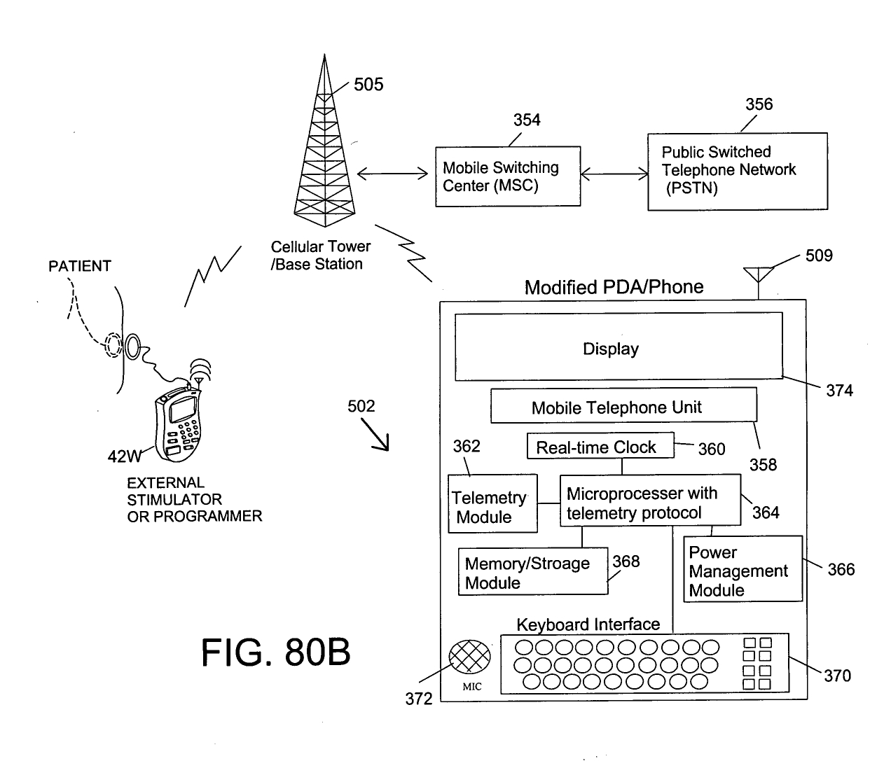

Shown in conjunction with FIGS. 80A and 80B the physician's remote communication's module is a Modified PDA/Phone 502 in this embodiment. The Modified PDA/Phone 502 is a microprocessor based device as shown in a simplified block diagram in FIGS. 76A and 76B. The PDA/Phone 502 is configured to accept PCM/CIA cards specially configured to fulfill the role of communication module 292 of the present invention. The Modified PDA/Phone 502 may operate under any of the useful software including Microsoft Window's based, Linux, Palm OS, Java OS, SYMBIAN, or the like.

The telemetry module 362 comprises an RF telemetry antenna 142 coupled to a telemetry transceiver and antenna driver circuit board which includes a telemetry transmitter and telemetry receiver. The telemetry transmitter and receiver are coupled to control circuitry and registers, operated under the control of microprocessor 364. Similarly, within stimulator a telemetry antenna 142 is coupled to a telemetry transceiver comprising RF telemetry transmitter and receiver circuit. This circuit is coupled to control circuitry and registers operated under the control of microcomputer circuit.

With reference to the telecommunications aspects of the disclosure, the communication and data exchange between Modified PDA/Phone 502 and external stimulator 42 operates on commercially available frequency bands. The 2.4-to-2.4853 GHz bands or 5.15 and 5.825 GHz, are the two unlicensed areas of the spectrum, and set aside for industrial, scientific, and medical (ISM) uses. Most of the technology today including this invention, use either the 2.4 or 5 GHz radio bands and spread-spectrum technology.

The telecommunications technology, especially the wireless internet technology, which this invention utilizes in one embodiment, is constantly improving and evolving at a rapid pace, due to advances in RF and chip technology as well as software development. Therefore, one of the intents of this invention is to utilize “state of the art” technology available for data communication between Modified PDA/Phone 502 and external stimulator 42. The intent of this invention is to use 3G technology for wireless communication and data exchange, even though in some cases 2.5G is being used currently.

For the system of the current invention, the use of any of the “3G” technologies for communication for the Modified PDA/Phone 502, is considered within the scope of the invention. Further, it will be evident to one of ordinary skill in the art that as future 4G systems, which will include new technologies such as improved modulation and smart antennas, can be easily incorporated into the system and method of current invention, and are also considered within the scope of the invention.

The present disclosure may be embodied in other specific forms without departing from the spirit or essential attributes thereof. It is therefore desired that the present embodiment be considered in all aspects as illustrative and not restrictive, reference being made to the appended claims rather than to the foregoing description to indicate the scope of the invention.

We claim:

1. A method of providing rectangular and/or complex electrical pulses to cortical tissues of a patient for at least one of providing improvement of functional recovery following stroke, treating or alleviating symptoms of tinnitus, essential tremor (ET) including Parkinson's disease, and depression comprising the steps of:
selecting a patient for providing said cortical electrical stimulation;
providing a pulse generator to generate rectangular and/or complex electrical pulses, wherein said pulse generator is one of: i) an external stimulator used in conjunction with an implanted stimulus-receiver; ii) an external stimulator used in conjunction with an implanted stimulus-receiver comprising a high value capacitor for storing electric charge; iii) a microstimulator; iv) a programmer-less implantable pulse generator (IPG) which is operable with a magnet; v) a programmable implantable pulse generator (IPG); vi) a combination implantable device comprising both a programmable implantable pulse generator (IPG) and a stimulus-receiver; or vii) a programmable implantable pulse generator (IPG) having a rechargeable battery;
providing at least one lead(s) with plurality of electrodes, wherein said at least one lead(s) is in electrical connection with said pulse generator, and with said plurality of electrodes adapted to be in proximity to or in contact with said cortical tissues; and
providing a programmer for at least one of activating, programming, or controlling said rectangular and/or complex electrical pulses provided to said cortical portion of patient's brain.

2. The method of claim 1, wherein the configuration of said plurality of electrode(s) for providing said electrical pulses is at least partly based on sensing electrical activity from the patient's cortical tissues.

3. The method of claim 1, wherein the placement of said plurality of electrodes on patient's cortex is based at least in part upon digital imaging techniques, such as fMRI, MRI, or CT scans.

4. The method of claim 1, wherein the placement of said plurality of electrodes on patient's cortex is based at least in part upon digital imaging techniques and sensing electrical activity from said cortical tissues of said patient.

5. The method of claim 1, wherein configuration between said plurality of electrodes for providing electrical pulses is changed between two or more different configurations.

6. The method of claim 1, wherein said rectangular and/or complex electrical pulses are provided according to predetermined/pre-packaged programs.

7. The method of claim 6, wherein said predetermined/pre-packaged programs can be modified.

8. The method of claim 1, wherein said pulse generator can further be remotely interrogated and/or programmed via a telemetry means over a wide area network, such as the internet.

9. The method of claim 1, wherein said pulses further comprise at least one of pulse amplitude approximately between 0.1 volt-25 volts; pulse width between 20 micro-seconds -5 milli-seconds; stimulation frequency between 5 Hz and 150 Hz, and/or blocking frequency between 100 and 1,000 Hz.

10. A method of providing rectangular and/or complex electrical pulses to cortical tissues of a patient for at least one of providing improvement of functional recovery following stroke, treating or alleviating symptoms of tinnitus, essential tremors including Parkinson's disease, and depression comprising the steps of:
selecting a patient for providing said electrical pulses to said cortical tissues;
providing a pulse generator for generating rectangular and/or complex electrical pulses, wherein said complex electrical pulses comprises at least one of multi-level pulses, biphasic pulses, non-rectangular pulses, or pulses with varying amplitude during the pulse;
implanting electrodes on the cortex, wherein placement of said electrodes is determined utilizing cortical sensing and/or a digital imaging techniques;
providing at least one lead(s) with plurality of electrodes, wherein said at least one lead(s) is in electrical connection with said pulse generation means, and said plurality of electrodes are adapted to be in proximity to cortical tissues; and
supplying said rectangular and/or complex electrical pulses to cortical tissues using said pulse generator means.

11. The method of claim 10, wherein said rectangular and/or complex electrical pulses are provided according to predetermined/pre-packaged programs.

12. The method of claim 11, wherein said predetermined/pre-packaged programs can be modified.

13. The method of claim 12, wherein the configuration between said plurality of electrodes for providing electrical pulses is changed between two or more different configurations.

14. The method of claim 12, wherein said pulse generator can further be remotely interrogated and/or programmed with a telemetry means over a wide area network, such as the internet.

15. The method of claim 12, wherein said electrical pulses further comprise pulse amplitude approximately between 0.1 volt-25 volts; pulse width between 20 micro-seconds-5 milli-seconds; stimulation frequency between 5 Hz and 150 Hz, and/or blocking frequency between 100 and 1,000 Hz.

16. A system for providing electrical pulses to the cortical region of the brain to provide therapy for at least one of stroke, tinnitus, essential tremor (ET) including Parkinson's disease, and depression comprising:
a pulse generator, wherein said pulse generator is selected from: i) an external stimulator used in conjunction with an implanted stimulus-receiver; ii) an external stimulator used in conjunction with an implanted stimulus-receiver comprising a high value capacitor for storing electric charge; iii) a microstimulator; iv) a programmer-less implantable pulse generator (IPG) which is operable with a magnet; v) a programmable implantable pulse generator (IPG); vi) a combination implantable device comprising both a programmable implantable pulse generator (IPG) and a stimulus-receiver; or vii) a programmable implantable pulse generator (IPG) having a rechargeable battery;
at least one lead(s) with plurality of electrodes, wherein said at least one lead(s) is in electrical connection with said pulse generator, and said plurality of electrodes adapted to be in proximity to or in contact with said cortical region; and
a programmer for at least one of activating, programming, controlling said electrical pulses provided to said cortical region of brain.

17. The system of claim 16, wherein said pulse generator further comprises at least two predetermined/pre-packaged programs stored in said pulse generator.

18. The system of claim 16, wherein said pulse generator can further be remotely interrogated and/or programmed via a telemetry means over a wide area network, such as the internet.

19. The system of claim 16, wherein said electrical pulses further comprise at least one of pulse amplitude approximately between 0.1 volt-25 volts; pulse width between 20 micro-seconds -5 milli-seconds; stimulation frequency between 5 Hz and 150 Hz, and/or blocking frequency between 100 and 1,000 Hz.

20. The system of claim 16, wherein said at least one lead(s) comprises plurality of electrodes in the form of a paddle electrodes or grid electrodes.
